(19) United States
(12) Patent Application Publication
(10) Pub. No.: US 2005/0192279 A1
(43) Pub. Date: Sep. 1, 2005

(54) Pyridazinones as antagonists of alpha4 integrins

(76) Inventors: Kent Barbay, Flourtown, PA (US); Alexey B. Dyatkin, Maple Glen, PA (US); Yong Gong, Warrington, PA (US); Wei He, Audubon, PA (US); Tamara A. Miskowski, Chalfont, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/054,190

(22) Filed: Feb. 9, 2005

Related U.S. Application Data
(60) Provisional application No. 60/543,372, filed on Feb. 10, 2004.

(51) Int. Cl. C07D 43/02; A61K 31/501; A61K 31/50

(52) U.S. Cl. 514/247; 514/252.03; 514/252.05; 544/238; 544/239

(57) ABSTRACT

The present invention relates to certain novel compounds of Formula (I): 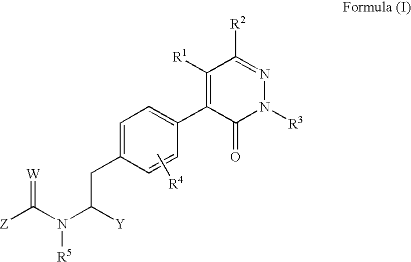 methods for preparing these compounds, compositions, intermediates and derivatives thereof and for the treatment of integrin mediated disorders.

### CROSS-REFERENCE TO RELATED APPLICATIONS

This Application claims priority to U.S. Provisional Patent Application No. 60/543,372, filed Feb. 10, 2004, which is hereby incorporated by reference in its entirety.

### STATEMENT REGARDING FEDERALLY SPONSORED RESEARCH OR DEVELOPMENT

The research and development of the invention described below was not federally sponsored.

### FIELD OF THE INVENTION

The present invention relates to certain novel compounds, methods for preparing compounds, compositions, intermediates and derivatives thereof and for treating integrin mediated disorders. More particularly, the pyridazinone compounds of the present invention are α4β1 and α4β7 integrin inhibitors useful for treating integrin mediated disorders.

### BACKGROUND OF THE INVENTION

The present invention relates to pyridazinone derivatives that inhibit α4 integrins. Many physiological processes require that cells come into close contact with other cells and/or extracellular matrix. Such adhesion events may be required for cell activation, migration, proliferation, and differentiation. Cell-cell and cell-matrix interactions are mediated through several families of cell adhesion molecules (CAMs) including the selectins, integrins, cadherins and immunoglobulins. CAMs play a role in both normal and pathophysiological processes. Therefore, the targeting of specific and relevant CAMs in certain disease conditions without interfering with normal cellular functions is essential for an effective and safe therapeutic agent that inhibits cell-cell and cell-matrix interactions.

The integrin superfamily is made up of structurally and functionally related glycoproteins consisting of α and β heterodimeric, transmembrane receptor molecules found in various combinations on nearly every mammalian cell type. α4β1 (“very late antigen-4” or VLA4) is an integrin expressed on nearly all leukocytes and is a key mediator of the cell-cell and cell-matrix interactions of these cell types. The ligands for α4β1 include vascular cell adhesion molecule-1 (VCAM-1) and the CS-1 domain of fibronectin (FN). VCAM-1 is a member of the Ig superfamily and is expressed in vivo on endothelial cells at sites of inflammation. VCAM-1 is produced by vascular endothelial cells in response to pro-inflammatory cytokines (A. J. H. Gearing and W. Newman, “Circulating adhesion molecules in disease.” Immunol. Today, 14, 506 (1993)). Therefore, α4β1 has become a therapeutic target for inflammatory conditions.

α4β7 is an integrin expressed on leukocytes and is a key mediator of leukocyte trafficking and homing in the gastrointestinal tract. The ligands for α4β7 include mucosal addressing cell adhesion molecule-1 (MAadCAM-1) and, upon activation of α4β7, VCAM-1 and fibronectin. MAdCAM-1 is a member of the Ig superfamily and is expressed in vivo on endothelial cells of gut-associated mucosal tissues of the small and large intestine.

Neutralizing anti-α4 antibodies or blocking peptides that inhibit the interaction between α4β1 and/or α4β7 and their ligands has proven efficacious both prophylactically and therapeutically in several animal models of disease including bronchial hyperresponsiveness in sheep and guinea pigs as models for the various phases of asthma (W. M. Abraham et al., “α4-Integrins mediate antigen-induced late bronchial responses and prolonged airway hyperresponsiveness in sheep.” J. Clin. Invest. 93, 776 (1993)); and adjuvant-induced arthritis in rats as a model of inflammatory arthritis (C. Barbadillo et al., “Anti-VLA-4 mAb prevents adjuvant arthritis in Lewis rats.” Arthr. Rheuma. (Suppl.), 36, 95 (1993)). There is evidence supporting a role for these integrins in other conditions such as diabetes, chronic colitis, tumor metastasis, and autoimmune thyroiditis.

There still remains a need for low molecular weight, specific inhibitors of α4β1 and α4β7-dependent cell adhesion that have improved pharmacokinetic and pharmacodynamic properties such as oral bioavailability and significant duration of action. Such compounds would prove useful for the treatment, prevention, or suppression of various pathologies mediated by α4β1 and α4β7 binding and cell adhesion and activation.

Therefore, it is an object of the present invention to provide pyridazinone compounds that are integrin inhibitors, in particular, inhibitors of α4β1 and α4β7, useful for treating inflammatory, immunological, and integrin-mediated disorders. It is another object of the invention to provide a process for preparing pyridazinone compounds, compositions, intermediates and derivatives thereof. It is a further object of the invention to provide methods for treating inflammatory and α4β1 and α4β7 integrin-mediated disorders.

### SUMMARY OF THE INVENTION

The present invention is directed to a compound of Formula (I) 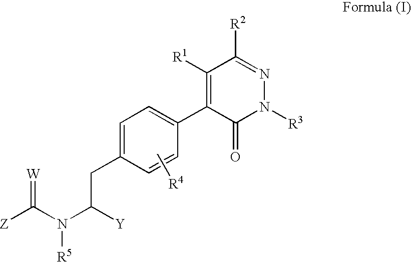
wherein
R¹ is a substituent independently selected from the group consisting of hydrogen, C₁₋₆alkyl, C₁₋₆alkoxy, aryl, heteroaryl, heterocyclyl, benzo fused heterocyclyl, benzo fused cycloalkyl, heteroaryl fused heterocyclyl, heteroaryl fused cycloalkyl, aryloxy, heteroaryloxy, heterocyclyloxy, cycloalkyloxy, —NR¹⁰R²⁰, halogen, hydroxy, and —S(C₁₋₆)alkyl; wherein C₁₋₆alkoxy is optionally substituted with one to four substituents independently selected from R^{a};
wherein R^{a} is independently selected from the group consisting of hydroxy(C₁₋₆)alkoxy, aryl, heteroaryl, heterocyclyl, cycloalkyl, (C₁₋₆)alkoxycarbonyl, carboxy, amino, alkylamino, dialkylamino, one to three halogen atoms, and hydroxy;
wherein R¹⁰ and R²⁰ are independently selected from the group consisting of hydrogen, C₁₋₆alkyl, allyl, halogenated C₁₋₆alkyl, hydroxy, hydroxy(C₁₋₄)alkyl, aryl, aryl(C₁₋₄)alkyl, and cycloalkyl; additionally, R¹⁰ and R²⁰ are optionally taken together with the atoms to which they are attached to form a five to seven membered monocyclic ring;
wherein the aryl and aryloxy substituents of R¹ are optionally substituted with a substituent independently selected from the group consisting of C₁₋₆alkyl, hydroxy(C₁₋₆)alkyl, aryl(C₁₋₆)alkyl, C₁₋₆alkoxy, aryl, heteroaryl, C₁₋₆alkoxycarbonyl, aryl(C₁₋₆)alkoxycarbonyl, C₁₋₆alkylcarbonyl, aminocarbonyl, alkylaminocarbonyl, dialkylaminocarbonyl, hydroxy, cyano, nitro, —SO₂(C₁₋₃)alkyl, —SO₂aryl, —SO₂heteroaryl, trifluoromethyl, trifluoromethoxy, and halogen;
and wherein the heteroaryl and heterocyclyl substituents of R¹ are optionally substituted with a substituent independently selected from the group consisting of one to three C₁₋₆alkyl substituents, C₁₋₆alkoxy, aryl, heteroaryl, one to three halogen atoms, and hydroxy;
R² is a substituent independently selected from the group consisting of hydrogen, C₁₋₆alkyl, C₁₋₆alkoxy, C₂₋₆alkenyloxy, hydroxy, amino, alkylamino, dialkylamino, and halogen;
wherein R¹ and R² are optionally taken together with the atoms to which they are attached to form a five to seven membered carbocyclic or heterocyclic ring;
R³ is a substituent independently selected from the group consisting of hydrogen, C₁₋₆alkyl, C₂₋₆alkenyl, C₂₋₆alkynyl, aryl, heteroaryl, heterocyclyl, and cycloalkyl; wherein alkyl, alkenyl, and alkynyl are optionally substituted with a substituent independently selected from the group consisting of aminocarbonyl, alkylaminocarbonyl, dialkylaminocarbonyl, aryl, heteroaryl, heterocyclyl, cycloalkyl, carboxy, one to three halogen atoms, hydroxy, and —C(═O)C₁₋₆alkyl;
R⁴ is independently selected from the group consisting of hydrogen, fluorine, chlorine, and methyl;
R⁵ is hydrogen or C₁₋₃alkyl, provided that R⁵ is C₁₋₃alkyl only when taken with Y and the atoms to which R⁵ and Y are attached to form a five to seven membered heterocycle;
Y is independently selected from the group consisting of hydroxymethyl, —C(═O)NH₂, —C(═O)NH(OH), —C(═O)NH(C₁₋₆alkyl), —C(═O)NH(hydroxy(C₁₋₆)alkyl), —C(═O)N(C₁₋₆alkyl)₂, —C(═O)NHSO₂(C₁₋₄)alkyl, carboxy, tetrazolyl, and —C(═O)C₁₋₆alkoxy; wherein said alkoxy is optionally substituted with one to two substituents independently selected from hydroxy, —NR³⁰R⁴⁰, heterocyclyl, heteroaryl, halogen, or —OCH₂CH₂OCH₃; wherein R³⁰ and R⁴⁰ are independently selected from the group consisting of hydrogen, C₁₋₆alkyl, hydroxy, and hydroxy(C₁₋₄)alkyl, and said R³⁰ and R⁴⁰ are optionally taken together with the atoms to which they are attached to form a five to seven membered monocyclic ring;
W is O or S;
Z is selected from the group consisting of hydrogen, C₁₋₆alkyl, C₁₋₆alkenyl, C₁₋₆alkynyl, C₁₋₆alkoxy, aryl, heteroaryl, cycloalkyl, heterocyclyl, cycloalkyloxy, polycycloalkyloxy, and aza-bridged polycycyl wherein aza-bridged polycycyl is optionally substituted with R^{d};
wherein alkyl and alkoxy are optionally substituted with one to three substituents independently selected from the group consisting of aryl, aryl(C₁₋₄)alkoxy, heteroaryl optionally substituted with one to three C₁₋₂alkyl substitutents or —C(═O)aryl, hydroxy, —C(═O)C₁₋₆alkyl, —NH₂, —NH(C₁₋₆alkyl), —N(C₁₋₆alkyl)₂, —NH(cycloalkyl) wherein said cycloalkyl is optionally spirofused to a heterocyclyl, —NHC(═O)aryl(C₁₋₄)alkoxy, —N(C₁₋₆alkyl)C(═O)aryl(C₁₋₄)alkoxy, —NHC(═O)heteroaryl(C₁₋₄)alkyl, —N(C₁₋₆alkyl)C(═O)heteroaryl(C₁₋₄)alkyl, —NHC(═O)aryl(C₁₋₄)alkyl, —N(C₁₋₆alkyl)C(═O)aryl(C₁₋₄)alkyl, —NHC(═O)(C₁₋₄)alkoxy, —N(C₁₋₆alkyl)C(═O)(C₁₋₄)alkoxy, —NHC(═O)NH₂, —N(C₁₋₄alkyl)C(═O)NH₂, —NHC(═O)NH(C₁₋₄)alkyl, —NHC(═O)N(C₁₋₄alkyl)₂, —NHSO₂aryl, —C(═O)NH₂, —C(═O)NH(C₁₋₆alkyl), —C(═O)N(C₁₋₆alkyl)₂, and halogen;
wherein the aryl and heteroaryl substituents of Z are optionally substituted with one to four substituents independently selected from the group consisting of C₁₋₄alkyl, hydroxyC₁₋₄alkyl, C₁₋₄alkoxy, hydroxy, halogen, nitro, carboxy, amino, alkylamino, dialkylamino, —SO₂(C₁₋₄)alkyl, and —C(═O)aryl; additionally, the heteroaryl is optionally substituted with oxo;
wherein the cycloalkyl and heterocyclyl substituents of Z are optionally substituted with one to four substituents independently selected from the group consisting of C₁₋₅alkyl, C₁₋₅alkylamino, di(C₁₋₅)alkylamino, —NH(cycloalkyl) wherein said cycloalkyl is optionally spirofused to a heterocyclyl, aminocarbonyl, —NHC(═O)C₁₋₄alkoxy, —N(C₁₋₆alkyl)C(═O)C₁₋₄alkoxy, —C(═O)(C₁₋₄)alkoxy, —NHC(═O)C₁₋₄alkyl, —N(C₁₋₆alkyl)C(═O)C₁₋₄alkyl, —C(═O)aryl(C₁₋₄)alkoxy, oxo, alkoxy, hydroxy, aryl(C₁₋₄)alkoxy, heteroaryl(C₁₋₄)alkoxy, heterocyclyl, heteroaryl optionally substituted with one to three C₁₋₂alkyl substituents, and aryl, wherein the aryl substituent is optionally substituted with one to four substituents independently selected from the group consisting of C₁₋₄alkyl, halogen, amino, alkylamino, dialkylamino, aryl, and heteroaryl;
wherein R^{d} is a substituent independently selected from the group consisting of (C₁₋₆)alkyl, —C(—O)(C₁₋₆)alkyl, —C(═O)(C₁₋₆)alkoxy, —S(═O)C₁₋₄alkyl, —SO₂C₁₋₄alkyl, —S(═O)aryl, and —SO₂aryl; wherein the alkyl and alkoxy portion of (C₁₋₆)alkyl, —C(═O)(C₁₋₆)alkyl, —C(═O)(C₁₋₆)alkoxy, —S(═O)C₁₋₄alkyl, and —SO₂C₁₋₄alkyl, are optionally substituted with one to three substitutents independently selected from the group consisting of C₁₋₃alkoxy, hydroxy, aryl, heteroaryl, and heterocyclyl; and wherein said aryl and heteroaryl are optionally substituted with one to five substituents independently selected from the group consisting of C₁₋₆alkyl, hydroxy(C₁₋₆)alkyl, C₁₋₆alkoxy, carboxy, hydroxy, cyano, nitro, amino, alkylamino, dialkylamino, —SO₂(C₁₋₃)alkyl, —SO₂aryl, —SO₂heteroaryl, trifluoromethyl, trifluoromethoxy, and halogen;
and an optical isomer, enantiomer, diastereomer, racemate, or pharmaceutically acceptable salt thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

The present invention is also directed to methods for producing the instant pyridazinone compounds and pharmaceutical compositions and medicaments thereof.

The present invention is further directed to methods for treating or ameliorating an α4 integrin-mediated disorder. In particular, the method of the present invention is directed to treating or ameliorating an α4 integrin mediated disorder such as, but not limited to multiple sclerosis, asthma, allergic rhinitis, allergic conjunctivitis, inflammatory lung disease, rheumatoid arthritis, septic arthritis, type I diabetes, organ transplantation rejection, restenosis, autologous bone marrow transplantation, inflammatory sequelae of viral infections, myocarditis, inflammatory bowel disease including ulcerative colitis and Crohn's disease, certain types of toxic and immune based nephritis, contact dermal hypersensitivity psoriasis, tumor metastasis, atherosclerosis and hepatitis.

### DETAILED DESCRIPTION OF THE INVENTION

An embodiment of the present invention includes compounds of Formula (I) 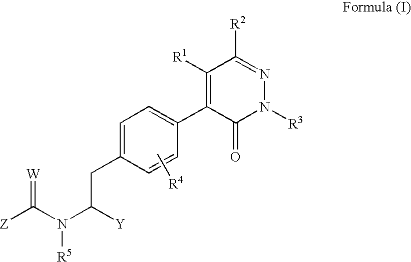
wherein:
R¹ is a substituent independently selected from the group consisting of hydrogen, C₁₋₆alkyl, C₁₋₆alkoxy, aryl, heteroaryl, heterocyclyl, benzo fused cycloalkyl, benzo fused heterocyclyl, heteroaryl fused heterocyclyl, heteroaryl fused cycloalkyl, aryloxy, heteroaryloxy, heterocyclyloxy, cycloalkyloxy, —NR¹⁰R²⁰, halogen, hydroxy, and —S(C₁₋₆)alkyl;
   wherein the alkoxy substituent of R¹ is optionally substituted with one to four substituents independently selected from R^{a}; wherein R^{a} is independently selected from the group consisting of aryl, heteroaryl, heterocyclyl, cycloalkyl, carboxy, amino, alkylamino, dialkylamino, hydroxy(C₁₋₆)alkoxy, one to three halogen atoms, and hydroxy;
   wherein R¹⁰ and R²⁰ are independently selected from the group consisting of hydrogen, C₁₋₆alkyl, allyl, halogenated C₁₋₆alkyl, and cycloalkyl; additionally, R¹⁰ and R²⁰ are optionally taken together with the atoms to which they are attached to form a five to seven membered monocyclic ring;
wherein the aryl and aryloxy substituents of R¹ are optionally substituted with a substituent independently selected from the group consisting of C₁₋₆alkyl, C₁₋₆alkoxy, aryl, heteroaryl, C₁₋₆alkylcarbonyl, aminocarbonyl, alkylaminocarbonyl, dialkylaminocarbonyl, hydroxy, cyano, nitro, —SO₂(C₁₋₃)alkyl, —SO₂aryl, trifluoromethyl, trifluoromethoxy, and halogen;
and wherein the heteroaryl and heterocyclyl substituents of R¹ are optionally substituted with a substituent independently selected from the group consisting of one to three C₁₋₆alkyl substituents, C₁₋₆alkoxy, aryl, heteroaryl, one to three halogen atoms, hydroxy C₁₋₆alkyl, and hydroxy; additionally, R¹ and R² are optionally taken together with the atoms to which they are attached to form a five to seven membered carbocyclic or heterocyclic ring.

An embodiment of the present invention includes compounds of Formula (I) wherein:
R¹ is selected from the group consisting of C₁₋₄alkyl, C₁₋₄alkoxy, aryl, heteroaryl, heterocyclyl, benzo fused heterocyclyl, aryloxy, heteroaryloxy, heterocyclyloxy, cycloalkyloxy, —NR¹⁰R²⁰, halogen, hydroxy, and —S(C₁₋₆)alkyl; wherein the alkoxy substutuent of R¹ is optionally substituted with one to three substituents independently selected from R^{a};
   wherein R^{a} is independently selected from the group consisting of heteroaryl, heterocyclyl, cycloalkyl, aryl, dialkylamino, hydroxy(C₁₋₆)alkoxy, one to three halogen atoms, and hydroxy;
   wherein R¹⁰ and R²⁰ are independently selected from the group consisting of hydrogen, C₁₋₆alkyl, allyl, and cycloalkyl;
wherein the aryl and aryloxy substituents of R¹ are optionally substituted with a substituent independently selected from the group consisting of C₁₋₆alkyl, C₁₋₆alkoxy, phenyl, heteroaryl, aminocarbonyl, alkylaminocarbonyl, dialkylaminocarbonyl, hydroxy, cyano, nitro, —SO₂(C₁₋₃)alkyl, —SO₂aryl, trifluoromethyl, trifluoromethoxy, and halogen;
and wherein the heteroaryl and heterocyclyl substituents of R¹ are optionally substituted with a substituent independently selected from the group consisting of one to three C₁₋₆alkyl groups, halogen, and hydroxy;
Additionally, R¹ and R² are optionally taken together with the atoms to which they are attached to form a five to seven membered carbocyclic or heterocyclic ring.

An even further embodiment of the present invention includes compounds of Formula (I) wherein:
R¹ is selected from the group consisting of ethyl, methoxy, ethoxy, 2-hydroxyeth-1-oxy, iso-propoxy, iso-butoxy, difluoromethoxy, 2,2,2-trifluoro-eth-1-oxy, benzyloxy, cyclopropylmethoxy, pyridin-3-ylmethoxy, (1-methyl)-pyrrolidinyl-3-oxy, cyclobutyloxy, cyclopentyloxy, cyclohexyloxy, indazol-1-yl, thiophen-3-yl, [1,3]benzodioxol-5-yl, (2-methyl)-imidazol-1-yl, (1-methyl)-piperidin-4-yloxy, 2-(morpholin-4-yl)-ethoxy, (4-bromo)-pyrazol-1-yl, N-pyrrolidinyl, (3,5-dimethyl)-pyrazol-1-yl, morpholin-4-yl, hydroxy, —(OCH₂CH₂)₂OH, phenyl (optionally substituted with a substituent independently selected from the group consisting of —SO₂Me, —C(═O)NH₂, —OCF₃, —CF₃, cyano, fluoro, and methoxy), amino, cyclopropylamino, allylamino, methylamino, hydroxy, chloro, and —SMe;
additionally, R¹ is optionally taken together with R² to form a 1,4-dioxanyl or a oxazinyl ring.

An even further embodiment of the present invention includes compounds of Formula (I) wherein:
R¹ is selected from the group consisting of methoxy, ethoxy, 2-hydroxyeth-1-oxy, iso-propoxy, iso-butoxy, difluoromethoxy, 2,2,2-trifluoro-eth-1-oxy, benzyloxy, cyclopropylmethoxy, pyridin-3-ylmethoxy, (1-methyl)-pyrrolidinyl-3-oxy, cyclobutyloxy, cyclopentyloxy, cyclohexyloxy, indazol-1-yl, thiophen-3-yl, [1,3]benzodioxol-5-yl, (2-methyl)-imidazol-1-yl, (1-methyl)-piperidin-4-yloxy, 2-(morpholin-4-yl)-ethoxy, (4-bromo)-pyrazol-1-yl, N-pyrrolidinyl, (3,5-dimethyl)-pyrazol-1-yl, morpholin-4-yl, hydroxy, —(OCH₂CH₂)₂OH, phenyl (optionally substituted with —SO₂Me, —C(═O)NH₂, —OCF₃, —CF₃, cyano, fluoro, or methoxy), cyclopropylamino, allylamino, and methylamino;
and wherein R¹ is optionally taken together with R² to form a 1,4-dioxanyl or a oxazinyl ring.

An embodiment of the present invention includes compounds of Formula (I) wherein:
R² is a substituent independently selected from the group consisting of hydrogen, C₁₋₄alkyl, C₁₋₄alkoxy, C₂₋₄alkenyloxy, hydroxy, amino, and halogen; wherein R¹ and R² are optionally taken together with the atoms to which they are attached to form a five to seven membered carbocyclic or heterocyclic ring.

An embodiment of the present invention includes compounds of Formula (I) wherein:
R² is a substituent independently selected from the group consisting of hydrogen, C₁₋₄alkyl, C₁₋₄alkoxy, hydroxy, amino, alkylamino, and halogen; wherein R² is optionally taken together with R¹ to form a 1,4-dioxanyl or an oxazinyl ring.

An embodiment of the present invention includes compounds of Formula (I) wherein:
R² is a substituent independently selected from the group consisting of hydrogen, C₁₋₄alkoxy, amino, and alkylamino; wherein R² is optionally taken together with R¹ to form a 1,4-dioxanyl or an oxazinyl ring.

Further embodiments of the present invention include compounds of Formula (I) wherein:
R³ is a substituent independently selected from the group consisting of hydrogen, C₁₋₆alkyl, aryl, heteroaryl, heterocyclyl, and cycloalkyl; wherein the alkyl substituent of R³ is optionally substituted with a substituent independently selected from the group consisting of —C(═O)NH₂, aryl, heteroaryl, heterocyclyl, cycloalkyl, carboxy, one to three halogen atoms, hydroxy, and —C(═O)C₁₋₆alkyl.

An embodiment of the present invention includes compounds of Formula (I) wherein:
R³ is a substituent independently selected from the group consisting of hydrogen, C₁₋₄alkyl, cycloalkyl, and aryl; wherein C₁₋₄alkyl is optionally substituted with a substituent independently selected from the group consisting of —C(═O)C₁₋₄alkyl, —C(═O)NH₂, carboxy, heterocyclyl, phenyl, cyclopropyl, hydroxy, and one to three fluorine atoms.

An embodiment of the present invention includes compounds of Formula (I) wherein:
R³ is a substituent independently selected from the group consisting of hydrogen, C₁₋₄alkyl, and phenyl; wherein C₁₋₄alkyl is optionally substituted with a substituent selected from —C(═O)C₁₋₄alkyl, —C(═O)NH₂, carboxy, morpholinyl, cyclopropyl, hydroxy, or one to three fluorine atoms.

An embodiment of the present invention includes compounds of Formula (I) wherein:
R³ is a substituent independently selected from the group consisting of hydrogen, methyl, ethyl, and phenyl; wherein methyl and ethyl are optionally substituted with a substituent independently selected from the group consisting of —C(═O)C₁₋₄alkyl, —C(═O)NH₂, carboxy, morpholinyl, cyclopropyl, hydroxy, and one to three fluorine atoms.

An embodiment of the present invention includes compounds of Formula (I) wherein:
R⁴ is independently selected from the group consisting of hydrogen, fluorine, and chlorine.

A further embodiment of the present invention includes compounds of Formula (I) wherein:
R⁴ is independently selected from hydrogen or fluorine.

A further embodiment of the present invention includes compounds of Formula (I) wherein:
R⁴ is hydrogen.

An embodiment of the present invention includes compounds of Formula (I) wherein:
R⁵ is hydrogen or C₁₋₃alkyl, provided that R⁵ is C₁₋₃alkyl only when taken with Y and the atoms to which R⁵ and Y are attached to form a five to seven membered heterocycle.

An embodiment of the present invention includes compounds of Formula (I) wherein:
R⁵ is hydrogen or methylene, provided that R⁵ is methylene only when taken with Y and the atoms to which R⁵ and Y are attached to form a five membered heterocycle.

A further embodiment of the present invention includes compounds of Formula (I) wherein:
R⁵ is hydrogen.

An even further embodiment of the present invention includes compounds of Formula (I) wherein:
Y is independently selected from the group consisting of hydroxymethyl, —C(═O)NH₂, —C(═O)NH(OH), —C(═O)NH(2-hydroxyeth-1-yl), carboxy, tetrazolyl, —C(═O)NHSO₂(C₁₋₄)alkyl, and —C(═O)C₁₋₆alkoxy; wherein said alkoxy is optionally substituted with one to two substituents independently selected from the group consisting of hydroxy, —NR³⁰R⁴⁰, heterocyclyl, heteroaryl, halogen, and —OCH₂CH₂OCH₃; wherein R³⁰ and R⁴⁰ are independently selected from the group consisting of hydrogen and C₁₋₆alkyl.

An even further embodiment of the present invention includes compounds of Formula (I) wherein:
Y is independently selected from the group consisting of carboxy, tetrazolyl, —C(═O)NH(2-hydroxyeth-1-yl) and —C(═O)C₁₋₄alkoxy; wherein said alkoxy is optionally substituted with one to two substituents independently selected from the group consisting of hydroxy, —NH₂, —NH(C₁₋₄)alkyl, —N(C₁₋₄alkyl)₂, heterocyclyl, halogen, and—OCH₂CH₂OCH₃.

An even further embodiment of the present invention includes compounds of Formula (I) wherein:
Y is independently selected from the group consisting of carboxy, 1H-tetrazol-5-yl, and —C(═O)C₁₋₄alkoxy; wherein said alkoxy is optionally substituted with a substituent independently selected from hydroxy, —NMe₂, morpholin-1-yl, chloro, or —OCH₂CH₂OCH₃.

An even further embodiment of the present invention includes compounds of Formula (i) wherein:
Y is independently selected from the group consisting of carboxy, 1H-tetrazol-5-yl, or —C(═O)ethoxy; wherein ethoxy is optionally substituted with hydroxy, chlorine, —NMe₂, and —OCH₂CH₂OCH₃.

An embodiment of the present invention includes compounds of Formula (I) wherein:
Z is independently selected from the group consisting of C₁₋₆alkyl, C₁₋₆alkenyl, C₁₋₆alkoxy, aryl, heteroaryl, cycloalkyl, heterocyclyl, polycycloalkyloxy, and aza-bridged polycycyl wherein aza-bridged polycycyl is optionally substituted with R^{d};
wherein the C₁₋₆alkyl substituent of Z is optionally substituted with one to three substituents independently selected from the group consisting of aryl, aryl(C₁₋₄)alkoxy, heteroaryl optionally substituted with one to three C₁₋₂alkyl substituents, hydroxy, —NH₂, —NH(C₁₋₆alkyl), —N(C₁₋₆alkyl)₂, —NH(cycloalkyl) wherein said cycloalkyl is optionally spirofused to a heterocyclyl, —NHC(═O)aryl(C₁₋₄)alkoxy, —N(C₁₋₆alkyl)C(═O)aryl(C₁₋₄)alkoxy, —NHC(═O)heteroaryl(C₁₋₄)alkyl, —N(C₁₋₆alkyl)C(═O)heteroaryl(C₁₋₄)alkyl, —NHC(═O)aryl(C₁₋₄)alkyl, —N(C₁₋₆alkyl)C(═O)aryl(C₁₋₄)alkyl, —NHC(═O)(C₁₋₄)alkoxy, —N(C₁₋₆alkyl)C(═O)(C₁₋₄)alkoxy, —NHC(═O)NH₂, —NHSO₂aryl, —C(═O)NH₂, —C(═O)NH(C₁₋₆alkyl), —C(═O)N(C₁₋₆alkyl)₂, and halogen;
wherein the aryl and heteroaryl substituents of Z are optionally substituted with one to four substituents independently selected from the group consisting of C₁₋₄alkyl, hydroxyC₁₋₄alkyl, C₁₋₄alkoxy, hydroxy, halogen, nitro, carboxy, amino, alkylamino, dialkylamino, —SO₂(C₁₋₄)alkyl, and —C(═O)aryl; additionally, the heteroaryl is optionally substituted with oxo;
wherein the cycloalkyl and heterocyclyl substituents of Z are optionally substituted with one to four substituents independently selected from the group consisting of C₁₋₅alkyl, amino, C₁₋₅alkylamino, di(C₁₋₅)alkylamino, —NH(cycloalkyl) wherein said cycloalkyl is optionally spirofused to a heterocyclyl, aminocarbonyl, —NHC(═O)C₁₋₄alkoxy, —N(C₁₋₆alkyl)C(═O)C₁₋₄alkoxy, —C(═O)(C₁₋₄)alkoxy, —C(═O)(C₁₋₄)alkyl, —C(═O)aryl(C₁₋₄)alkoxy, oxo, alkoxy, hydroxy, aryl(C₁₋₄)alkoxy, and aryl; wherein said aryl is optionally substituted with one to four substituents independently selected from the group consisting of C₁₋₄alkyl, halogen, amino, alkylamino, and dialkylamino.

Another embodiment of the present invention includes compounds of Formula (I) wherein:
Z is independently selected from the group consisting of C₁₋₆alkyl, C₁₋₆alkenyl, C₁₋₆alkoxy, aryl, heteroaryl, cycloalkyl, heterocyclyl, and aza-bridged polycycyl wherein aza-bridged polycycyl is optionally substituted with R^{d};
wherein the C₁₋₆alkyl substituent of Z is optionally substituted with one to three substituents independently selected from the group consisting of aryl, heteroaryl optionally substituted with one to three C₁₋₂alkyl substituents, hydroxy, aryl(C₁₋₄)alkoxy, —C(═O)C₁₋₆alkyl, —NH(C₁₋₆alkyl), —N(C₁₋₆alkyl)₂, —NH(cycloalkyl) wherein said cycloalkyl is optionally spirofused to a heterocyclyl, —NHC(═O)aryl(C₁₋₄)alkoxy, —N(C₁₋₆alkyl)C(═O)aryl(C₁₋₄)alkoxy, —NHC(═O)heteroaryl(C₁₋₄)alkyl, —N(C₁₋₆alkyl)C(═O)heteroaryl(C₁₋₄)alkyl, —N(C₁₋₆alkyl)C(═O)aryl(C₁₋₄)alkyl, —NHC(═O)(C₁₋₄)alkoxy, —N(C₁₋₆alkyl)C(═O)(C₁₋₄)alkoxy, —NHC(═O)NH₂, —NHSO₂aryl, and halogen;
wherein the aryl and heteroaryl substituents of Z are optionally substituted with one to four substituents independently selected from the group consisting of C₁₋₄alkyl, halogen, nitro, and —SO₂(C₁₋₄)alkyl;
wherein the cycloalkyl and heterocyclyl substituents of Z are optionally substituted with a substituent independently selected from the group consisting of one to four C₁₋₄alkyl substituents, —C(═O)NH₂, —C(═O)NH(C₀₋₁₄)alkyl, amino, (C₁₋₄)alkylamino, —NH(cycloalkyl) wherein said cycloalkyl is optionally spirofused to a heterocyclyl, —NHC(═O)C₁₋₄alkoxy, —C(═O)(C₁₋₄)alkyl, —C(═O)aryl(C₁₋₄)alkoxy, oxo, alkoxy, hydroxy, aryl(C₁₋₄)alkoxy, and aryl; wherein said aryl is optionally substituted with one to four substituents independently selected from the group consisting of C₁₋₄alkyl and halogen.

An even further embodiment of the present invention includes compounds of Formula (I) wherein:
Z is independently selected from the group consisting of C₁₋₄alkyl, C₁₋₄alkenyl, C₁₋₄alkoxy, aryl, heteroaryl, cycloalkyl, heterocyclyl, and aza-bridged polycycyl wherein aza-bridged polycycyl is optionally substituted with R^{d};
wherein the C₁₋₄alkyl substituent of Z is optionally substituted with one to three substituents independently selected from the group consisting of aryl, heteroaryl optionally substituted with one to two methyl substituents, —NH₂, —NH(C₁₋₆alkyl), —NH(cycloalkyl), aryl(C₁₋₄)alkoxy, —N(methyl)C(═O)aryl(C₁₋₄)alkoxy, —N(methyl)C(═O)heteroaryl(C₁₋₄)alkyl, —N(methyl)C(═O)aryl(C₁₋₄)alkyl, —NHC(═O)C₁₋₄alkoxy, —N(methyl)C(═O)C₁₋₄alkoxy, and —NHC(═O)NH₂;
wherein the aryl and heteroaryl substituents of Z are optionally substituted with one to four substituents independently selected from the group consisting of C₁₋₄alkyl, halogen, and —SO₂(C₁₋₄)alkyl; additionally, the heteroaryl is optionally substituted with oxo;
wherein the cycloalkyl and heterocyclyl substituents of Z are optionally substituted with one to four substituents independently selected from the group consisting of C₁₋₄alkyl, aminocarbonyl, amino, C₁₋₄alkylamino, —NH(cycloalkyl) wherein said cycloalkyl is optionally spirofused to a heterocyclyl, —NHC(═O)C₁₋₄alkoxy, —N(C₁₋₆alkyl)C(═O)C₁₋₄alkoxy, —C(═O)(C₁₋₄)alkoxy, aryl(C₁₋₄)alkoxy, and —C(═O)aryl(C₁₋₄)alkoxy.

An even further embodiment of the present invention includes compounds of Formula (I) wherein:
Z is independently selected from the group consisting of C₁₋₄alkyl, C₁₋₄alkenyl, C₁₋₄alkoxy, phenyl, pyrrolyl, pyridinyl, C₃₋₆cycloalkyl, tetrahydropyranyl, and 2-aza-bicyclo[2.2.2.]-octanyl wherein 2-aza-bicyclo[2.2.2]-octanyl is optionally substituted with R^{d};
wherein the C₁₋₄alkyl is optionally substituted with one to three substituents independently selected from the group consisting of phenyl, thiophenyl, pyrrolyl optionally substituted with one to two methyl substituents, —NH₂, —N H(C₁₋₆alkyl), —NH(cycloalkyl), —N(methyl)C(═O)benzyloxy, —N (methyl)C(═O)thiophenylmethyl, —N(methyl)C(═O)phenylethyl, —NHC(═O)ₜ-butoxy, —N(methyl)C(═O)ₜ-butoxy, and —NHC(═O)NH₂;
wherein phenyl and the heteroaryl substituents of Z are optionally substituted with one to four substituents independently selected from the group consisting of methyl, fluorine, chlorine, and —SO₂methyl; additionally, the heteroaryl is optionally substituted with oxo;
wherein the C₃₋₆cycloalkyl substituent of Z is optionally substituted with a substituent independently selected from the group consisting of one to four methyl substituents, —C(═O)NH₂, —C(═O)NH(i-propyl), —NHcycloalkyl wherein said cycloalkyl is optionally spirofused to a heterocyclyl, (i-propyl)amino, amino, phenyl(C₁₋₄)alkoxy; additionally, the tetrahydropyranyl substituent of Z is optionally spiro-fused to a heterocyclyl.

An even further embodiment of the present invention includes compounds of Formula (I) wherein:
Z is independently selected from the group consisting of 2,6-dichloro-phenyl, 2-chloro-4-methanesulfonyl-phenyl, 2-chloro-5-fluoro-phenyl, 2,6-dichloro-pyridinyl-N-oxide, 3,5-dichloro-pyridin-4-yl, 1-phenyl-2-methyl-prop-1-yl, —CH(i-propyl)-N(Me)C(═O)CH₂thiophenyl, —CH(i-propyl)-NHcyclohexyl, —CH(i-propyl)-(2,5-dimethyl)-pyrrol-1-yl, —CH(l-propyl)-N(Me)ₜ-butoxy, —CH(i-propyl)-N H-t-butoxy, —CH(i-propyl)-NH(Me), (1-aminocarbonyl)-cycloprop-1-yl, (1-1-propylamino)cycloprop-1-yl, and 2-methyl-prop-2-en-1-yl.

An even further embodiment of the present invention includes compounds of Formula (I) wherein:
R^{d} is a substituent independently selected from the group consisting of (C₁₋₆)alkyl, —C(═O)(C₁₋₆)alkyl, —C(═O)(C₁₋₆)alkoxy, —S(═O)C₁₋₄alkyl, —SO₂C₁₋₄alkyl, —S(═O)aryl, and —SO₂aryl;
   wherein the alkyl and alkoxy portion of (C₁₋₆)alkyl, —C(═O)(C₁₋₆)alkyl, —C(═O)(C₁₋₆)alkoxy, —S(═O)C₁₋₄alkyl, and —SO₂C₁₋₄alkyl are optionally substituted with one to three substitutents independently selected from the group consisting of C₁₋₃alkoxy, hydroxy, aryl, heterocyclyl, and heteroaryl; wherein said aryl and heteroaryl are optionally substituted with one to five substituents independently selected from the group consisting of C₁₋₆alkyl, hydroxy(C₁₋₆)alkyl, C₁₋₆alkoxy, carboxy, hydroxy, cyano, nitro, —SO₂(C₁₋₃)alkyl, —SO₂aryl, —SO₂heteroaryl, trifluoromethyl, trifluoromethoxy, and halogen.

An even further embodiment of the present invention includes compounds of Formula (I) wherein:
R^{d} is a substituent independently selected from the group consisting of —C(═O)(C₁₋₆)alkyl, —C(═O)(C₁₋₆)alkoxy, —S(═O)C₁₋₄alkyl, —SO₂C₁₋₄alkyl, —S(═O)aryl, and —SO₂aryl; wherein the alkyl and alkoxy portion of (C₁₋₆)alkyl, —C(═O)(C₁₋₆)alkyl, —C(═O)(C₁₋₆)alkoxy, —S(═O)C₁₋₄alkyl, and —SO₂C₁₋₄alkyl is optionally substituted with one to three substitutents independently selected from the group consisting of C₁₋₃alkoxy, aryl, and heteroaryl.

An embodiment of the present invention includes compounds of Formula (I) wherein:
R^{d} is a substituent independently selected from the group consisting of —C(═O)(C₁₋₆)alkyl, —C(═O)(C₁₋₆)alkoxy, —SO₂C₁₋₄alkyl, and —SO₂aryl;
   wherein the alkyl and alkoxy portion of —C(═O)(C₁₋₆)alkyl, —C(═O)(C₁₋₆)alkoxy, and —SO₂C₁₋₄alkyl is optionally substituted with a substitutent independently selected from the group consisting of C₁₋₃alkoxy, aryl, and heteroaryl.

An embodiment of the present invention includes compounds of Formula (I) wherein:
R^{d} is independently selected from the group consisting of —C(═O)(C₁₋₆)alkyl, —C(═O)(C₁₋₆)alkoxy, and —SO₂phenyl;
   wherein the alkyl and alkoxy portion of —C(═O)(C₁₋₆)alkyl and —C(═O)(C₁₋₆)alkoxy is optionally substituted with a substitutent independently selected from the group consisting of methoxy, phenyl, tetrazolyl, furanyl, and thiophenyl.
and an optical isomer, enantiomer, diastereomer, racemate, or pharmaceutically acceptable salt thereof.

One embodiment of the present invention is directed to compounds of Formula (Ia) wherein the substituents are as previously defined (including the previously listed preferred substitutions for R¹, R², R³, W, Y, and Z in any combination). Examples of embodiments of the present invention are shown in Table 1:

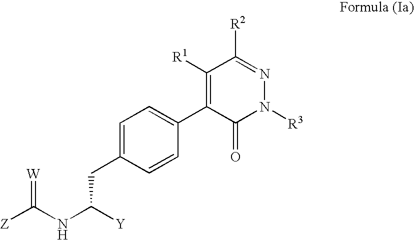

wherein R¹, R², R³, W, Y, and Z are dependently selected from the group consisting of:

TABLE I

| Cpd | R¹ | R² | R³ | Y | W | Z |
|---|---|---|---|---|---|---|
| 134 | OCH₃ | H | CH₃ | CO₂H | O | (2,6-Cl₂)phenyl |
| 215 | OCH₃ | H | CH₃ | CO₂H | O | (S)—CH(i-Pr)-2,5- |
| | | | | | | dimethyl- |
| | | | | | | pyrrol-1-yl |

Another embodiment of the present invention is further directed to a compound of Formula (Ib), wherein the substituents are as previously defined (including the previously listed preferred substitutions for R¹, R², R³, R⁵, W, Y, and Z in any combination). Examples of embodiments of the present invention are shown in Table II:

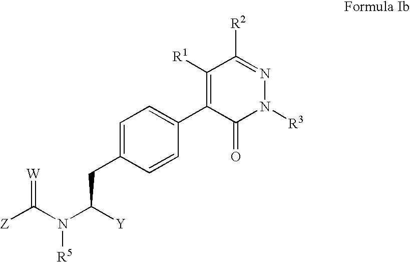

wherein R¹, R², R³, R⁵, W, Y, and Z are dependently selected from the group consisting of:

TABLE II

| | | | | | | | |
|---|---|---|---|---|---|---|---|
| Cpd | R¹ | R² | R³ | R⁵ | Y | W | Z |

| | | | | | | |
|---|---|---|---|---|---|---|
| *114 | OCH₃ | H | CH₃ | —CH₂OC(═O)— | O | (2,6- |
| | | | | | | Cl₂)phenyl |

*indicates a prodrug

Another embodiment of the present invention is further directed to a compound of Formula (Ic) wherein the substituents are as previously defined (including the previously listed preferred substitutions for R¹, R², R³, W, Y, and Z in any combination). Examples of embodiments of the present invention are as shown in Table III:

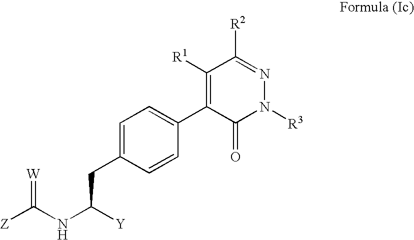

wherein R¹, R R³, W, Y, and Z are dependently selected from the group consisting of:

TABLE III

| | | | | | | | Stereo chem |
|---|---|---|---|---|---|---|---|
| Cpd | R¹ | R² | R³ | Y | W | Z | of Z |
| *1 | OCH₃ | H | CH₃ | —CO₂Et | O | (2,6-Cl₂)phenyl | |
| *2 | OCH₃ | H | CH₃ | —C(═O)O(CH₂)₂OH | O | (2-Cl, 5-F)phenyl | |
| 3 | OCH₃ | H | CH₃ | CO₂H | O | 1-(i-Pr-amino)-cycloprop-1-yl | |
| 4 | OEt | H | CH₃ | CO₂H | O | (2,6-Cl₂)phenyl | |
| 5 | OCH₃ | H | —CH₂C(═O)NH₂ | CO₂H | O | (2,6-Cl₂) phenyl | |
| 6 | —OCH₂CH₂O— | | CH₃ | CO₂H | O | (2,6-Cl₂)phenyl | |
| 7 | —(OCH₂CH₂)₂OH | H | CH₃ | CO₂H | O | (2,6-Cl₂)phenyl | |
| 8 | (2-OH)eth-1-oxy | H | CH₃ | CO₂H | O | (2,6-Cl₂)phenyl | |
| 9 | OCH₃ | H | CH₃ | CO₂H | O | (3,5-Cl₂)pyridin-4-yl-N-oxide | |
| 10 | OCH₃ | H | 2-(morpholin- | CO₂H | O | (2,6-Cl₂)phenyl | |
| | | | 4-yl)eth-1-yl | | | | |
| 11 | OCH₃ | H | —CH₂CO₂H | CO₂H | O | (2,6-Cl₂)phenyl | |
| 12 | (1-Me)pyrrolidin-3- | H | CH₃ | CO₂H | O | (2,6-Cl₂)phenyl | |
| | yloxy | | | | | | |
| 13 | —NHCH₂CH₂O— | | CH₃ | CO₂H | O | (2,6-Cl₂)phenyl | |
| 14 | (4-SO₂Me)phenyl | H | CH₃ | CO₂H | O | (2,6-Cl₂)phenyl | |
| 15 | OCH₃ | H | (2-OH)eth-1-yl | CO₂H | O | (2,6-Cl₂)phenyl | |
| 16 | 4-(C(═O)NH₂)phenyl | H | CH₃ | CO₂H | O | (2,6-Cl₂)phenyl | |
| 17 | OCH₃ | H | CH₃ | CO₂H | O | (2,6-Cl₂)phenyl | |
| 18 | NHMe | H | —CH₂C(═O)Me | CO₂H | O | (2,6-Cl₂)phenyl | |
| 19 | OCH₃ | H | CH₃ | CO₂H | O | —CH(i-Pr)N(Me)C(═O)CH₂- | R |
| | | | | | | thiophen-3-yl | |
| 20 | morpholin-4-yl | H | CH₃ | CO₂H | O | (2,6-Cl₂)phenyl | |
| 21 | 2-(morpholin- | H | CH₃ | CO₂H | O | (2,6-Cl₂)phenyl | |
| | 4-yl)ethoxy | | | | | | |
| 22 | (2-morpholin- | H | (2-OH)eth-1-yl | CO₂H | O | (2,6-Cl₂)phenyl | |
| | 4-yl)ethoxy | | | | | | |
| 23 | (1-Me)piperidin-4- | H | (2-OH)eth-1-yl | CO₂H | O | (2,6-Cl₂)phenyl | |
| | yloxy | | | | | | |
| 24 | i-Propoxy | H | (2-OH)eth-1-yl | CO₂H | O | (2,6-Cl₂)phenyl | |
| 25 | pyridin-3-ylmethoxy | H | CH₃ | CO₂H | O | (2,6-Cl₂)phenyl | |
| 26 | (2-OH)eth-1-yl | H | (2-OH)eth-1-yl | CO₂H | O | (2,6-Cl₂)phenyl | |
| 27 | [1,3]benzodioxol- | H | CH₃ | CO₂H | O | (2,6-Cl₂)phenyl | |
| | 5-yl | | | | | | |
| 28 | OCH₃ | H | CH₃ | CO₂H | O | —CH(i-Pr)NH(cyclohexyl) | |
| 29 | morpholin-4-yl | H | Et | CO₂H | O | (2,6-Cl₂)phenyl | |
| 30 | pyridin-3- | H | (2-OH)eth-1-yl | CO₂H | O | (2,6-Cl₂)phenyl | |
| | ylmethoxy | | | | | | |
| 31 | OCH₃ | H | CH₃ | CO₂H | O | (2-Cl, 4-SO₂Me)phenyl | |
| 32 | (2-Me)imidazol-1-yl | H | CH₃ | CO₂H | O | (2,6-Cl₂)phenyl | |
| 33 | OCH₃ | H | CH₃ | CO₂H | O | —CH(i-Pr)(2,5-Me₂)-pyrrol-1-yl | d |
| *34 | OCH₃ | H | CH₃ | —C(═O)O(CH₂)₂NMe₂ | O | (2,6-Cl₂)phenyl | |
| 35 | cyclopropylmethoxy | H | (2-OH)eth-1-yl | CO₂H | O | (2,6-Cl₂)phenyl | |
| 36 | —NH(allyl) | H | —CH₂C(═O)Me | CO₂H | O | (2,6-Cl₂)phenyl | |
| 37 | (2,2,2-F₃)eth- | H | CH₃ | CO₂H | O | (2,6-Cl₂)phenyl | |
| | 1-oxy | | | | | | |
| 38 | OCH₃ | H | CH₃ | CO₂H | O | 1-(C(═O)NH₂)-cycloprop-1-yl | |
| 39 | OCH₃ | H | (2,2,2-F₃)eth-1-yl | CO₂H | O | (2,6-Cl₂)phenyl | |
| 40 | OCH₃ | H | CH₃ | CO₂H | O | 1-(cyclohexylamino)-cycloprop-1-yl | |
| 41 | cyclohexyloxy | H | CH₃ | CO₂H | O | (2,6-Cl₂)phenyl | |
| 42 | OCH₃ | H | CH₃ | CO₂H | O | 4-(i-Pr-amino)-tetrahydro-pyran-4-yl | |
| 43 | cyclopentyloxy | H | (2-OH)eth-1-yl | CO₂H | O | (2,6-Cl₂)phenyl | |
| 44 | OCH₃ | H | CH₃ | CO₂H | | (2-Cl,5-F)phenyl | |
| 45 | OCH₃ | H | CH₃ | CO₂H | | 2-methyl-prop-2-en-1-yl | |
| 46 | NH₂ | H | CH₃ | CO₂H | O | (2,6-Cl₂)phenyl | |
| 47 | (4-OMe)phenyl | H | CH₃ | CO₂H | O | (2,6-Cl₂)phenyl | |
| 48 | OCH₃ | H | CH₃ | CO₂H | O | —CH(i-Pr)N(Me)(C(═O)OtBu) | S |
| 49 | Cl | H | CH₃ | CO₂H | O | (2,6-Cl₂)phenyl | |
| 50 | OCH₃ | H | CH₃ | CO₂H | O | —CH(Me)N(Me)C(═O)CH₂- | R |
| | | | | | | thiophen-3-yl | |
| 51 | pyrrolidin-1-yl | H | CH₃ | CO₂H | O | (2,6-Cl₂)phenyl | |
| 52 | benzyloxy | H | CH₃ | CO₂H | O | (2,6-Cl₂)phenyl | |
| 53 | cyclobutyloxy | H | (2-OH)eth-1-yl | CO₂H | O | (2,6-Cl₂)phenyl | |
| 54 | OCH₃ | H | CH₃ | CO₂H | O | —CH(i-Pr)NH(Me) | S |
| 55 | OCH₃ | H | CH₃ | CO₂H | O | (3,5-Cl₂)pyridin-4-yl | |
| 56 | OCH₃ | H | Ph | CO₂H | O | (2,6-Cl₂)phenyl | |
| 57 | OCH₃ | H | CH₃ | CO₂H | O | 1-Ph-2-methyl-prop-1-yl | d |
| 58 | OCH₃ | H | CH₃ | CO₂H | O | —CH(i-Pr)NHC(═O)OtBu | d |
| 59 | (4-F)phenyl | H | CH₃ | CO₂H | O | (2,6-Cl₂)phenyl | |
| 60 | cyclopentyloxy | H | CH₃ | CO₂H | O | (2,6-Cl₂)phenyl | |
| 61 | morpholin-4-yl | H | (2-OH)eth-1-yl | CO₂H | O | (2,6-Cl₂)phenyl | |
| 62 | OH | H | CH₃ | CO₂H | O | (3,5-Cl₂)pyridin-4-yl | |
| 63 | phenyl | H | CH₃ | CO₂H | O | (2,6-Cl₂)phenyl | |
| 64 | (3-CF₃)phenyl | H | CH₃ | CO₂H | O | (2,6-Cl₂)phenyl | |
| 65 | (4-SO₂Me)phenyl | H | cyclopropylmethyl | CO₂H | O | (2,6-Cl₂)phenyl | |
| 67 | (4-CN)phenyl | H | CH₃ | CO₂H | O | (2,6-Cl₂)phenyl | |
| 68 | OCH₃ | H | CH₃ | CO₂H | O | 1-(methylamino)- | 1S, |
| | | | | | | 2-(benzyloxy)-prop-1-yl | 2R |
| 69 | (3,5-Me₂)pyrazol- | H | CH₃ | CO₂H | O | (2,6-Cl₂)phenyl | |
| | 1-yl | | | | | | |
| 70 | OCH₃ | H | CH₃ | CO₂H | O | —CH(i-Pr)NH(4- | d |
| | | | | | | (1,4-dioxaspiro[4.5]decan-1-yl)) | |
| 71 | (3-OCF₃)phenyl | H | CH₃ | CO₂H | O | (2,6-Cl₂)phenyl | |
| 72 | (1-Me)pyrrolidinyl- | H | (2-OH)eth-1-yl | CO₂H | O | (2,6-Cl₂)phenyl | |
| | 3-oxy | | | | | | |
| 74 | i-Propoxy | H | CH₃ | CO₂H | O | (2,6-Cl₂)phenyl | |
| *75 | OCH₃ | H | CH₃ | —C(═O)O(CH₂)₂Cl | O | (2,6-Cl₂)phenyl | |
| 76 | OCH₃ | H | CH₃ | CO₂H | O | (2-Cl)pyridin-3-yl | |
| 77 | OCH₃ | H | CH₃ | CO₂H | O | —CH(i-Pr)NHSO₂(2-NO₂)Phenyl | d |
| 78 | SCH₃ | H | t-Bu | CO₂H | O | (2,6-Cl₂)phenyl | |
| 79 | indazol-1-yl | H | CH₃ | CO₂H | O | (2,6-Cl₂)phenyl | |
| 80 | OCH₃ | H | CH₃ | CO₂H | O | 1-(2,6-Me₂-pyrrol-1-yl)- | |
| | | | | | | cycloprop-1-yl | |
| 81 | OCH₃ | H | CH₃ | CO₂H | O | —CH(i-Pr)NH(C(═O)OtBu | R |
| 82 | (4-Br)pyrazol-1-yl | H | CH₃ | CO₂H | O | (2,6-Cl₂)phenyl | |
| 83 | OCH₃ | H | H | CO₂H | O | (2,6-Cl₂)phenyl | |
| 84 | OCH₃ | H | CH₃ | CO₂H | O | pyrrol-2-yl | |
| 85 | OCH₃ | H | t-Bu | CO₂H | O | (2,6-Cl₂)phenyl | |
| 86 | cyclopropylmethoxy | H | CH₃ | CO₂H | O | (2,6-Cl₂)phenyl | |
| 87 | OCH₃ | H | CH₃ | CO₂H | O | —CH(i-Pr)pyrrol-1-yl | R |
| 88 | indazol-1-yl | H | (2-OH)eth-1-yl | CO₂H | O | (2,6-Cl₂)phenyl | |
| 89 | OCH₃ | H | CH₃ | CO₂H | O | 2-(phenylethylcarbonyl)-2- | S |
| | | | | | | aza-bicyclo[2.2.2]-octan-yl | |
| 90 | OCH₃ | H | CH₃ | CO₂H | O | —CH(i-Pr)N(Me)C(═O)(CH₂)₂Ph | R |
| *91 | OCH₃ | H | CH₃ | —CO₂(CH₂CH₂O)₂Me | O | (2,6-Cl₂)phenyl | |
| 92 | —NH(cyclopropyl) | H | —CH²C(═O)t-Bu | CO₂H | O | (2,6-Cl₂)phenyl | |
| 93 | Cl | H | cyclopropylmethyl | CO₂H | O | (2,6-Cl₂)phenyl | |
| 94 | (4-Br)pyrazol-1-yl | H | (2-OH)eth-1-yl | CO₂H | O | (2,6-Cl₂)phenyl | |
| 95 | OH | H | CH₃ | CO₂H | O | (2,6-Cl₂)phenyl | |
| 96 | OCH₃ | H | CH₃ | CO₂H | O | —CH₂N(Me)C(═O)OBn | |
| *97 | OCH₃ | H | CH₃ | —C(═O)O(CH₂)₂OH | O | (2,6-Cl₂)phenyl | |
| 98 | OCH₃ | H | CH₃ | CO₂H | O | 4-(cyclohexylamino)- | |
| | | | | | | tetrahydropyran-4-yl | |
| 99 | OCH₃ | H | CH₃ | CO₂H | O | 2-aza-bicyclo[2.2.2]-octan-1-yl | S |
| 100 | OCH₃ | H | CH₃ | CO₂H | O | 2-(3-methyl-but-1-ylcarbonyl)-2- | S |
| | | | | | | aza-bicyclo[2.2.2]-octan-1-yl | |
| 101 | Et | H | CH₃ | CO₂H | O | (2,6-Cl₂)phenyl | |
| 102 | OCH₃ | H | CH₃ | CO₂H | O | 2-(1H-tetrazolylmethylcarbonyl)- | S |
| | | | | | | 2-aza-bicyclo[2.2.2]-octan-1-yl | |
| 103 | OCH₃ | H | CH₃ | CO₂H | O | 2-(phenylmethoxycarbonyl)- | S |
| | | | | | | 2-aza-bicyclo[2.2.2]-octan-1-yl | |
| 105 | thiophen-3-yl | H | CH₃ | CO₂H | O | (2,6-Cl₂)phenyl | |
| 106 | OCH₃ | H | CH₃ | 1H-tetrazol-5-yl | O | (2,6-Cl₂)phenyl | |
| 107 | OCH₃ | H | CH₃ | CO₂H | O | —CH(i-Pr)NHC(═O)CH₂thiophen-3-yl | R |
| 108 | OCH₃ | H | CH₃ | CO₂H | O | 2-(phenylsulfonyl)- | S |
| | | | | | | 2-aza-bicyclo[2.2.2]-octan-1-yl | |
| 109 | CH₃ | H | CH₃ | CO₂H | O | (2,6-Cl₂)phenyl | |
| 110 | OCH₃ | H | CH₃ | CO₂H | O | (1-Me)-pyrrol-2-yl | |
| 111 | —O(i-Bu) | H | CH₃ | CO₂H | O | —O(i-Bu) | |
| 112 | OCHF₂ | H | CH₃ | CO₂H | O | (2,6-Cl₂)phenyl | |
| 113 | OCH₃ | H | CH₃ | CO₂H | O | 2-(thiophen3-ylmethylcarbonyl)- | S |
| | | | | | | 2-aza-bicyclo[2.2.2]-octan-1-yl | |
| 115 | OCH₃ | H | CH₃ | CO₂H | O | 2-(furan-2-ylethylcarbonyl)- | S |
| | | | | | | 2-aza-bicyclo[2.2.2]-octan-1-yl | |
| 116 | OCH₃ | H | CH₃ | CO₂H | O | 4-NH₂-tetrahydropyran-4-yl | |
| 117 | OCH₃ | H | Bn | CO₂H | O | (2,6-Cl₂)phenyl | |
| *118 | OCH₃ | H | CH₃ | —C(═O)O(CH₂)₂morpholin- | O | (2,6-Cl₂)Phenyl | |
| | | | | 1-yl | | | |
| *119 | OCH₃ | H | CH₃ | —C(═O)NH(CH₂)₂OH | O | (2,6-Cl₂)phenyl | |
| 120 | OCH₃ | H | (2-OH)eth-1-yl | CO₂H | O | —O(t-Bu) | |
| 121 | —CH₂C(═O)OEt | H | CH₃ | CO₂H | O | (2,6-Cl₂)phenyl | |
| 122 | OCH₃ | H | CH₃ | CO₂H | O | 2-(1H-imidazol-4-ylethylcarbonyl)- | S |
| | | | | | | 2-aza-bicyclo[2.2.2]-octan-1-yl | |
| 123 | OCH₃ | H | CH₃ | CO₂H | O | —(i-Pr)CH(NHMe)— | R |
| 124 | OCH₃ | H | CH₃ | CO₂H | O | 2-methyl-prop-1-yl | |
| 125 | OCH₃ | H | CH₃ | CO₂H | O | 2-(2-methoxy-eth-1-ylcarbonyl)-2- | S |
| | | | | | | aza-bicyclo[2.2.2]-octan-1-yl | |
| 126 | OCH₃ | H | CH₃ | CO₂H | O | 2-(2-t-butoxycarbonyl)- | S |
| | | | | | | 2-aza-bicyclo[2.2.2]-octan-1-yl | |
| 127 | OCH₃ | H | CH₃ | CO₂H | O | 2-methyl-1-hydroxy-prop-1-yl | S |
| 128 | OCH₃ | H | 2-(morpholin- | CO₂H | O | 1-methylamino-2-benzyloxy-prop-1-yl | 1S, |
| | | | 4-yl)-eth-1-yl | | | | 2R |
| 129 | OCH₃ | H | CH₃ | CO₂H | O | 2-methyl-1-hydroxy-prop-1-yl | R |
| 130 | OCH₃ | H | CH₃ | CO₂H | O | (7-OMe)chromen-2-one-3-yl | |
| 131 | 5-(thiophen-2- | H | (2-OH)eth-1-yl | CO₂H | O | (2,6-Cl₂)phenyl | |
| | yl)pyrazol-1-yl | | | | | | |
| 132 | OCH₃ | H | CH₃ | CO₂H | O | 1-(4-F-phenyl)-cyclopent-1-yl | |
| 133 | OCH₃ | H | CH₃ | CO₂H | O | 1-{{4-[1-Me, 4-OMe-pyridazin-5- | |
| | | | | | | one]-phenyl}-1-carboxy-eth- | |
| | | | | | | 1-ylaminocarbonyl}-cycloprop-1-yl | |
| 135 | OCH₃ | H | CH₃ | CO₂H | O | 2-(methyl)-2- | S |
| | | | | | | aza-bicyclo[2.2.2]-octan-1-yl | |
| 136 | OCH₃ | H | CH₃ | CO₂H | O | (2,2,3,3-Me₄)cycloprop-1-yl | |
| 137 | OCH₃ | H | CH₃ | CO₂H | O | (4-CO₂H)phenyl | |
| 138 | OCH₃ | H | CH₃ | CO₂H | O | (2-NH₂, 4,6-Me₂)pyridin-3-yl | |
| 139 | OCH₃ | H | CH₃ | CO₂H | O | 2-(2-(piperidin-4-yl)-eth-1-ylcarbonyl)-2- | S |
| | | | | | | aza-bicyclo[2.2.2]-octan-1-yl | |
| 140 | OCH₃ | H | CH₃ | CO₂H | S | (2,6-Cl₂)phenyl | |
| 141 | OCH₃ | H | CH₃ | CH₂OH | O | (2,6-Cl₂)phenyl | |
| 142 | OEt | H | t-Bu | CO₂H | O | (2,6-Cl₂)phenyl | |
| 143 | OCH₃ | H | CH₃ | CO₂H | O | —C(═O)i-Pr | |
| 144 | OCH₃ | H | CH₃ | CO₂H | O | (3,5-Me₂)isoxazol-4-yl | |
| 145 | OCH₃ | H | CH₃ | CO₂H | O | thiophen-3-ylmethyl | |
| 146 | OCH₃ | H | CH₃ | CO₂H | O | 1-(i-Propylamino)-cycloprop-1-yl | |
| 147 | OCH₃ | H | CH₃ | CO₂H | O | (5-Me)isoxazol-4-yl | |
| 148 | 5-(thiophen-2- | H | CH₃ | CO₂H | O | (2,6-Cl₂)phenyl | |
| | yl)pyrazol-1-yl | | | | | | |
| 150 | (2-NMe₂)ethoxy | H | CH₃ | CO₂H | O | (2,6-Cl₂)phenyl | |
| *151 | OCH₃ | H | CH₃ | —CO₂Me | O | (2,6-Cl₂)phenyl | |
| 152 | CH₂CO₂H | H | CH₃ | CO₂H | O | (2,6-Cl₂)phenyl | |
| 153 | OCH₃ | H | CH₃ | CO₂H | O | —CH(i-Pr)NH(i-Pr) | R |
| 154 | OCH₃ | H | 2-(morpholin- | CO₂H | O | —CH(1-OH-eth-1-yl)NHC(═O)Ot-Bu | 1S, |
| | | | 4-yl)eth-1-yl | | | | 2R |
| 155 | NMe₂ | H | t-Bu | CO₂H | O | (2,6-Cl₂)phenyl | |
| 156 | NHMe | H | CH₃ | CO₂H | O | (2,6-Cl₂)phenyl | |
| 157 | OCH₃ | H | CH₃ | CO₂H | O | —CH₂N(Me)C(═O)Ot-Bu | |
| 158 | H | H | CH₃ | CO₂H | O | (2,6-Cl₂)phenyl | |
| 159 | OCH₃ | H | CH₃ | CO₂H | O | —CH(i-Pr)NH₂ | |
| 160 | [1,2,4 | H | t-Bu | CO₂H | O | (2,6-Cl₂)Phenyl | |
| | triazol-1-yl | | | | | | |
| 161 | OCH₃ | H | CH₃ | CO₂H | O | 2-(C(═O)OBn)pyrrolidin-2-yl | |
| 162 | (2-Cl)ethylamino | H | CH₃ | CO₂H | O | (2,6-Cl₂)phenyl | |
| 163 | OCH₃ | H | CH₃ | CO₂H | O | 1H-pyrimadin-2,4-dione-6-yl | |
| 164 | OCH₃ | H | CH₃ | CO₂H | O | 1-(benzyloxycarbonyl)piperidin-4-yl | |
| 165 | OCH₃ | H | cyclohexyl | CO₂H | O | (2,6-Cl₂)phenyl | |
| 166 | OCH₃ | H | CH₃ | CO₂H | O | pyrrolidin-2-yl | d |
| 167 | OCH₃ | H | 2-(morpholin- | CO₂H | O | 2-hydroxy-1-(t- | 1R, |
| | | | 4-yl)eth-1-yl | | | butoxycarbonylamino)-prop-1-yl | 2S |
| 168 | OH | H | H | CO₂H | | (2,6-Cl₂)phenyl | |
| 169 | OCH₃ | H | CH₃ | CO₂H | O | (2,6-Cl₂)pyridin-2-yl | |
| 170 | OCH₃ | H | CH₃ | CO₂H | O | (4-hydroxymethyl)phenyl | |
| 171 | OCH₃ | H | CH₃ | CO₂H | O | neopentyloxy | |
| 172 | OCH₃ | H | CH₃ | CO₂H | O | benzyloxy | |
| 173 | OCH₃ | H | CH₃ | CO₂H | O | —CH₂NMe₂ | |
| 174 | OCH₃ | H | CH₃ | CO₂H | O | 2-(3-hydroxy- | S |
| | | | | | | 3-methyl-prop-1-ylcarbonyl)- | |
| | | | | | | 2-aza-bicyclo[2.2.2]-octan-1-yl | |
| 175 | OCH₃ | H | CH₃ | CO₂H | O | —O(i-Bu) | |
| 176 | OCH₃ | H | CH₃ | —C(═O)NH(OH) | O | (2,6-Cl₂)phenyl | |
| 177 | OCH₃ | H | CH₃ | CO₂H | O | 1-(t-Butoxycarbonylamino)-cycloprop-1-yl | |
| 178 | OCH₃ | H | CH₃ | CO₂H | O | OMe | |
| 179 | OCH₃ | H | 2-(morpholin- | CO₂H | O | —CH(i-Pr)NH(i-Pr) | R |
| | | | 4-yl)eth-1-yl | | | | |
| 180 | OCH₃ | H | CH₃ | CO₂H | O | adamantan-1-yloxy | |
| 181 | OCH₃ | H | CH₃ | CO₂H | O | —CH(i-Pr)NH₂ | R |
| *182 | OCH₃ | H | CH₃ | —CO₂Me | O | 2-(2-(2-phenyl-eth-1-ylcarbonyl)-2- | S |
| | | | | | | aza-bicyclo[2.2.2]-octan-1-yl | |
| 183 | OCH₃ | H | CH₃ | CO₂H | O | t-butoxy | |
| 184 | OCH₃ | H | CH₃ | CO₂H | O | i-Propoxy | |
| 185 | OCH₃ | H | CH₃ | CO₂H | O | 1-hydroxy-1-methyl-eth-1-yl | |
| 186 | OCH₃ | H | CH₃ | CO₂H | O | 4-(t-butoxycarbonyl)-tetrahydropyran-4-yl | |
| 187 | OCH₃ | H | CH₃ | CO₂H | O | indazol-3-yl | |
| 188 | OCH₃ | H | CH₃ | CO₂H | O | (2-OMe,4-NH₂,5-Cl)phenyl | |
| 189 | OCH₃ | H | CH₃ | CO₂H | O | —CH(i-Pr)NHcyclohexyl | S |
| 190 | NH₂ | H | CH₃ | CO₂H | O | t-butoxy | |
| 191 | OCH₃ | H | CH₃ | CO₂H | O | (2-OH)pyridin-3-yl | |
| 192 | OCH₃ | H | CH₃ | —C(═O)NHSO₂Me | O | (2,6-Cl₂)phenyl | |
| 193 | OCH₃ | H | CH₃ | CO₂H | O | (1-Me)-1H-pyridin-2-one-3-yl | |
| 194 | OCH₃ | H | (2-OH)eth-1-yl | —CH₂OH | O | (2,6-Cl₂)phenyl | |
| 195 | OCH₃ | H | CH₃ | CO₂H | O | (1-Boc)pyrrolidin-2-yl | d |
| 196 | (4-Me)phenoxy | H | t-Bu | CO₂H | O | (2,6-Cl₂)phenyl | |
| 197 | NH₂ | H | Ph | CO₂H | O | (2,6-Cl₂)phenyl | |
| 198 | OCH₃ | H | CH₃ | CO₂H | O | piperidin-4-yl | |
| *199 | OCH₃ | H | (2-OH)eth-1-yl | —C(═O)O(OCH₂)₂OH | O | (2,6-Cl₂)phenyl | |
| 200 | OCH₃ | H | CH₃ | CO₂H | O | (3-Cl)thiophen-2-yl | |
| 201 | OCH₃ | H | CH₃ | CO₂H | O | thiophen-2-ylmethoxy | |
| 202 | OCH₃ | H | CH₃ | CO₂H | O | (N-t-butoxycarbonyl)piperidin-4-yl | |
| 203 | OCH₃ | H | CH₃ | CO₂H | O | (2,4,6-Me₃)benzyloxy | |
| 204 | OCH₃ | H | CH₃ | CO₂H | O | (2,6-Cl₂)benzyloxy | |
| 205 | OCH₃ | H | CH₃ | CO₂H | O | 3H-imidazol-4-yl | |
| 206 | OCH₃ | H | CH₃ | CO₂H | O | 3,3-Me₂-but-1-yl | |
| *207 | OCH₃ | H | CH₃ | —CO₂CH₂tBu | O | (2,6-Cl₂)phenyl | |
| 208 | OCH₃ | H | CH₃ | CO₂H | O | cyclohexyloxy | |
| 209 | OCH₃ | H | CH₃ | CO₂H | O | (1-Ph)eth-1-oxy | R |
| 210 | OCH₃ | H | 2-(morpholin- | CO₂H | O | —CH(i-Pr)NH₂ | R |
| | | | 4-yl)eth-1-yl | | | | |
| 211 | OCH₃ | H | CH₃ | CO₂H | O | —CH(Me)(2,5-Me₂,4- | d |
| | | | | | | phenylcarbonyl)-pyrrol-1-yl | |
| *212 | OCH₃ | H | (2-OH)eth-1-yl | —C(═O)O(CH₂)₂OH | O | O(t-Bu) | |
| 213 | OCH₃ | H | CH₃ | CO₂H | O | —CH(i-Pr)-2,5-dimethylpyrrol-1-yl | R |
| 214 | OCH₃ | H | CH₃ | CO₂H | O | —CH(i-Pr)-2,5-dimethylpyrrol-1-yl | S |
| 216 | OCH₃ | H | CH₃ | CO₂H | O | —C(Me₂)(t-butoxycarbonylamino) | |
| 217 | OCH₃ | H | CH₃ | CO₂H | O | 1-hydroxy-cycloprop-1-yl | |
| 218 | OCH₃ | H | CH₃ | CO₂H | O | —C(Me₂)(i-propylamino) | |
| 219 | OCH₃ | H | CH₃ | CO₂H | O | cyclohexylamino | |
| 220 | OCH₃ | H | CH₃ | CO₂H | O | —C(Me₂)(1,4-dioxaspiro[4.5]dec- | |
| | | | | | | 8-ylamino) | |
| 221 | OCH₃ | H | CH₃ | CO₂H | O | —C(Me₂)(methylamino) | |
| 222 | OCH₃ | H | CH₃ | CO₂H | O | 1-(t-butoxycarbonylamino)- | |
| | | | | | | cyclohex-1-yl | |
| 223 | OCH₃ | H | CH₃ | CO₂H | O | 1-(t-butoxycarbonylamino)- | |
| | | | | | | cyclopent-1-yl | |
| 224 | OCH₃ | H | CH₃ | CO₂H | O | 1-(1,4-dioxaspiro[4.5]dec- | |
| | | | | | | 8-ylamino)-cycloprop-1-yl | |
| 225 | OCH₃ | H | CH₃ | CO₂H | O | 1-(cyclopentylamino)-cycloprop-1-yl | |
| 226 | OCH₃ | H | CH₃ | CO₂H | O | 1-(diethylamino)-cycloprop-1-yl | |
| 227 | OCH₃ | H | CH₃ | CO₂H | O | 1-(methycarbonylamino)-cycloprop-1-yl | |
| 228 | OCH₃ | H | —CH₂C(═O)Me | CO₂H | O | (2,6-Cl₂)phenyl | |
| 229 | OCH₃ | H | CH₃ | CO₂H | O | —C(Me₂)NHC(═O)NH₂ | |
| 230 | OCH₃ | H | CH₃ | CO₂H | O | 1-(phenylmethoxy)-cycloprop-1-yl | |
| *231 | OCH₃ | H | (2-OH)eth-1-yl | —C(═O)O(CH₂)₂OH | | t-butoxy | |
| *232 | NH₂ | H | CH₃ | —C(═O)O(CH₂)₂OH | | (2,6-Cl₂)phenyl | |

*indicates a prodrug
d = a diastereomeric mixture

A preferred embodiment of the present invention includes the representative compounds of Table IV.

TABLE IV
Cpd

| | |
|---|---|
| 2 | 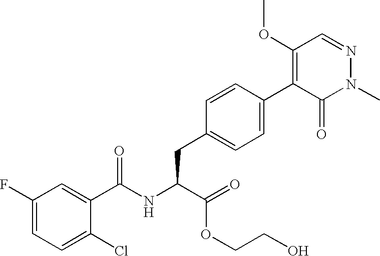 |
| 7 | 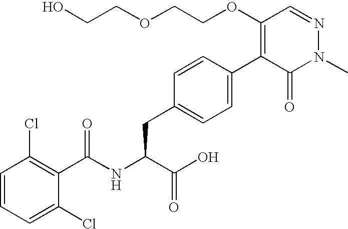 |
| 12 | 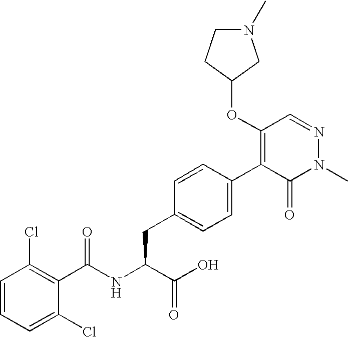 |
| 14 | 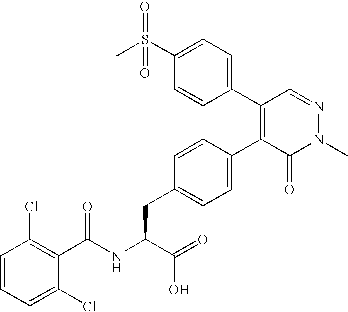 |
| 15 | 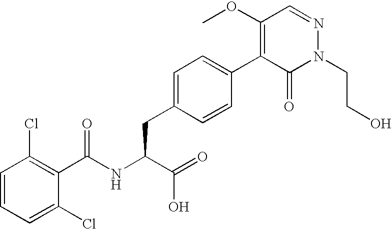 |
| 17 | 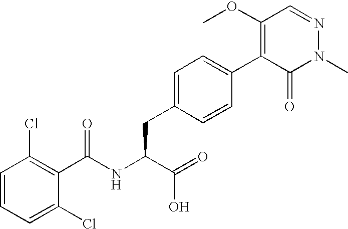 |
| 19 | 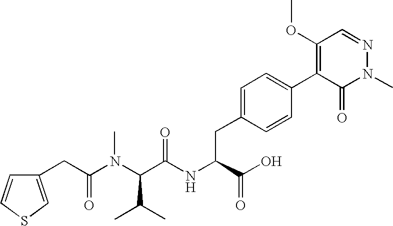 |
| 20 | 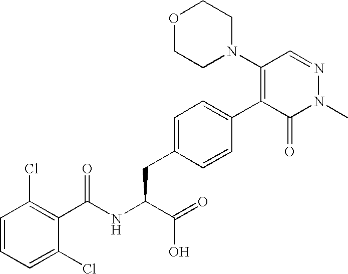 |
| 28 | 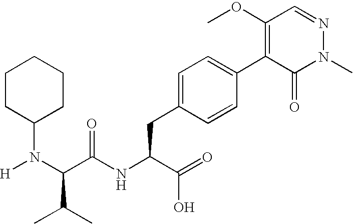 |
| 31 | 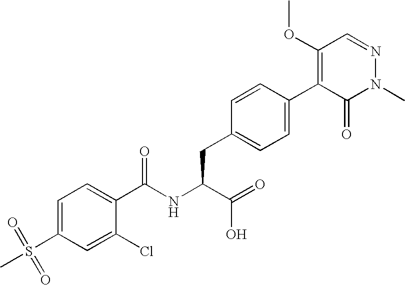 |
| 32 | 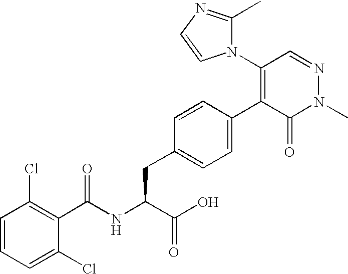 |
| 44 | 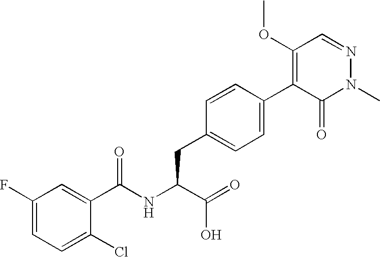 |
| 89 | 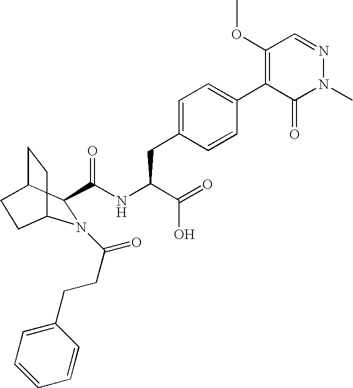 |
| 97 | 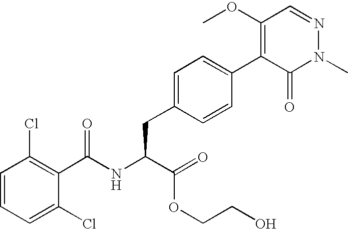 |
| 103 | 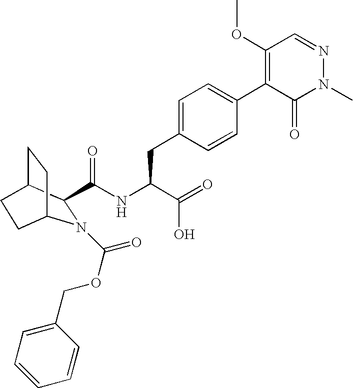 |
| 108 | 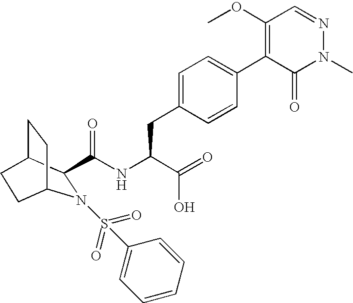 |
| 113 | 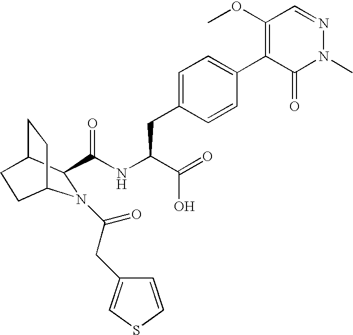 |
| 120 | 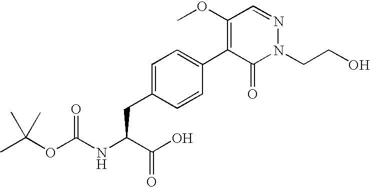 |
| 146 | 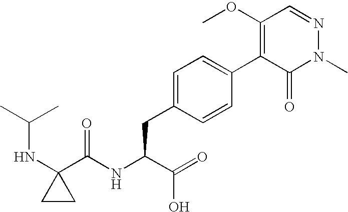 |
| | and |
| 212 | 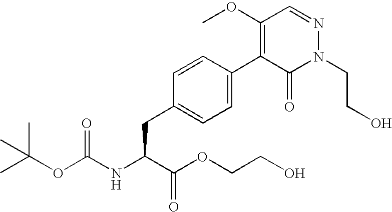 |

The compounds of the present invention, and preferably those compounds illustrated in Table IV, may be converted into pharmaceutically acceptable prodrugs using reagents and techniques known to those skilled in the art. A preferred prodrug derivative for the compounds of Table IV is a 2-hydroxyethyl ester. The preparation of 2-hydroxyethyl esters is demonstrated in Example 30 herein.

The compounds of the present invention may also be present in the form of pharmaceutically acceptable salts. For use in medicine, the salts of the compounds of this invention refer to non-toxic “pharmaceutically acceptable salts” (Ref. International J. Pharm., 1986, 33, 201-217; J. Pharm. Sci., 1997 (Jan), 66, 1, 1). Other salts may, however, be useful in the preparation of compounds according to this invention or of their pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. Where the processes for the preparation of the compounds according to the invention give rise to mixtures of stereoisomers, these isomers may be separated by conventional techniques such as preparative chromatography. The compounds may be prepared in racemic form or as individual enantiomers or diasteromers by either stereospecific synthesis or by resolution. The compounds may, for example, be resolved into their component enantiomers or diasteromers by standard techniques, such as the formation of stereoisomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers and enantiomers thereof are encompassed within the scope of the present invention.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

As used herein, unless otherwise noted, “alkyl” and “alkoxy” whether used alone or as part of a substituent group refers to straight and branched carbon chains having 1 to 8 carbon atoms or any number within this range. Similarly, alkenyl and alkynyl groups include straight and branched chain alkenes and alkynes having 2 to 8 carbon atoms or any number within this range, wherein an alkenyl chain has at least one double bond in the chain and an alkynyl chain has at least one triple bond in the chain. Alkoxy radicals are oxygen ethers formed from the previously described straight or branched chain alkyl groups.

As used herein, unless otherwise noted “oxo” whether used alone or as part of a substituent group refers to an O=to either a carbon or a sulfur atom. For example, phthalimide and saccharin are examples of compounds with oxo substituents.

The term “cycloalkyl,” as used herein, refers to an optionally substituted, stable, saturated or partially saturated monocyclic or bicyclic ring system containing from 3 to 8 ring carbons and preferably 5 to 7 ring carbons. Examples of such cyclic alkyl rings include cyclopropyl, cyclobutyl, cyclopentyl, cyclohexyl or cycloheptyl.

The term “benzo fused cycloalkyl” shall mean an optionally substituted stable ring system wherein one of the rings is phenyl and the other is a cycloalkyl as previously defined. Examples of such benzo fused cycloalkyl includes, but is not limited to, indane, dihydronaphthalene, and 1,2,3,4-tetrahydronaphthalene.

The term “polycycloalkyl” as used herein refers to an optionally substituted stable, saturated or partially saturated tricyclic or tetracyclic ring system containing from 8 to 12 carbons. Examples of such polycyclic alkyl rings include adamantyl.

The term “heterocyclyl” as used herein refers to an optionally substituted, stable, saturated or partially saturated 5 or 6 membered monocyclic or bicyclic ring systems which consists of carbon atoms and from one to three heteroatoms selected from N, O or S. Examples of heterocyclyl groups include, but are not limited to, pyrrolinyl (including 2H-pyrrole, 2-pyrrolinyl or 3-pyrrolinyl), pyrrolidinyl, dioxolanyl, 2-imidazolinyl, imidazolidinyl, 2-pyrazolinyl, pyrazolidinyl, piperidinyl, dioxanyl, morpholinyl, dithianyl, thiomorpholinyl or piperazinyl. The heterocyclyl group may be attached at any heteroatom or carbon atom which results in the creation of a stable structure.

The term “benzo fused heterocycle” or the radical “benzo fused heterocyclyl” as used herein refers to a optionally substituted, stable ring structure wherein one of the rings is phenyl and the other is stable, saturated or partially saturated 5 or 6 membered monocyclic or 8 to 10 membered bicyclic ring system which consists of carbon atoms and from one to three heteroatoms selected from N, O, or S. Examples of benzo fused heterocyclyl groups include, but are not limited to, indoline, isoindoline, and 1,2,3,4-tetrahydroquinoline.

The term “aza-bridged polycyclyl” refers to an optionally substituted stable ring structure of the formula: 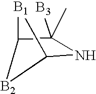
wherein B₁ and B₂ are independently selected from the group consisting of C₁₋₂alkylene and C₂alkenylene and B₃ is hydrogen or a C₁₋₄alkyl. Preferably, B₃ is hydrogen. The amine of the aza-bicyclic is the preferred point of attachment of the R^{d} substituent.

The term “aryl”, as used herein, refers to optionally substituted aromatic groups comprising a stable six membered monocyclic or ten membered bicyclic aromatic ring system which consists of carbon atoms. Examples of aryl groups include, but are not limited to, phenyl or naphthalenyl.

The term “heteroaryl” as used herein represents a stable five or six membered monocyclic aromatic ring system or a nine or ten membered benzo-fused heteroaromatic ring system which consists of carbon atoms and from one to three heteroatoms selected from N, O or S. The heteroaryl group may be attached at any heteroatom or carbon atom which results in the creation of a stable structure.

The term “heteroaryl fused heterocyclyl” as used herein represents a optionally substituted stable bicyclic ring structure in which one ring is an aromatic five or six membered ring which consists of carbon atoms and from one to three heteroatoms selected from N, O or S and the second ring is a stable, saturated or partially saturated 5 or 6 membered ring which consists of carbon atoms and from one to three heteroatoms selected from N, O or S.

The term “heteroaryl fused cycloalkyl” as used herein represents an optionally substituted stable bicyclic ring structure in which one ring is an aromatic five or six membered ring which consists of carbon atoms and from one to three heteroatoms selected from N, O or S and the other ring is a saturated or partially saturated ring containing from 3 to 8 ring carbons and preferably 5 to 7 ring carbons.

The term “arylalkyl” means an alkyl group substituted with an aryl group (e.g., benzyl, phenethyl). The term “arylalkoxy” indicates an alkoxy group substituted with an aryl group (e.g., benzyloxy, phenethoxy, etc.). Similarly, the term “aryloxy” indicates an oxy group substituted with an aryl group (e.g., phenoxy).

Whenever the term “alkyl” or “aryl” or either of their prefix roots appear in a name of a substituent (e.g., aralkyl, alkylamino) it shall be interpreted as including those limitations given above for “alkyl” and “aryl.” Designated numbers of carbon atoms (e.g., C₁₋₆) shall refer independently to the number of carbon atoms in an alkyl or cycloalkyl moiety or to the alkyl portion of a larger substituent in which alkyl appears as its prefix root.

The term “cycloalkyloxy” and “polycycloalkyloxy” whether used alone or as part of a substituent group, denotes an oxygen ether radical of the above described cycloalkyl or polycyloalkyl groups.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The pyridazinone compounds of the present invention are useful α4 integrin receptor antagonists and, more particularly, α4β1 and α4β7 integrin receptor antagonists for treating a variety of integrin mediated disorders that are ameliorated by inhibition of the α4β1 and α4β7 integrin receptor including, but not limited to, inflammatory, autoimmune and cell-proliferative disorders.

Illustrative of the invention is a pharmaceutical composition comprising a pharmaceutically acceptable carrier and any of the compounds described above. Also illustrative of the invention is a pharmaceutical composition made by mixing any of the compounds described above and a pharmaceutically acceptable carrier. A further illustration of the invention is a process for making a pharmaceutical composition comprising mixing any of the compounds described above and a pharmaceutically acceptable carrier. The present invention also provides pharmaceutical compositions comprising one or more compounds of this invention in association with a pharmaceutically acceptable carrier.

An example of the invention is a method for the treatment of integrin mediated disorders in a subject in need thereof comprising administering to the subject a therapeutically effective amount of any of the compounds or pharmaceutical compositions described above. Also included in the invention is the use of a compound of Formula (I) for the preparation of a medicament for treating an integrin mediated disorder in a subject in need thereof.

Further exemplifying the invention is the method for the treatment of integrin mediated disorders, wherein the therapeutically effective amount of the compound is from about 0.01 mg/kg/day to about 120 mg/kg/day.

In accordance with the methods of the present invention, the individual components of the pharmaceutical compositions described herein can be administered separately at different times during the course of therapy or concurrently in divided or single combination forms. The instant invention is therefore to be understood as embracing all such regimes of simultaneous or alternating treatment and the term “administering” is to be interpreted accordingly.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, that is being sought by a researcher, veterinarian, medical doctor, or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

The utility of the compounds to treat integrin mediated disorders can be determined according to the procedures herein. The present invention therefore provides a method for the treatment of integrin mediated disorders in a subject in need thereof which comprises administering any of the compounds as defined herein in a quantity effective to inhibit the α4β1 and α4β7 integrin receptor including, but not limited to, inflammatory, autoimmune and cell-proliferative disorders.

The ability of the compounds of Formula I to antagonize the actions of VLA4 and/or α4β7 integrin makes them useful for preventing or reversing the symptoms, disorders or diseases induced by the binding of VLA4 and or α4β7 to their various respective ligands. Thus, these antagonists will inhibit cell adhesion processes including cell activation, migration, proliferation and differentiation. Accordingly, another aspect of the present invention provides a method for the treatment (including prevention, alleviation, amelioration or suppression) of diseases or disorders or symptoms mediated by VLA-4 and/or α4β7 binding and cell adhesion and activation, which comprises administering to a mammal an effective amount of a compound of Formula I. Such diseases, disorders, conditions or symptoms are for example (1) multiple sclerosis, (2) asthma, (3) allergic rhinitis, (4) allergic conjunctivitis, (5) inflammatory lung diseases, (6) rheumatoid arthritis, (7) septic arthritis, (8) type I diabetes, (9) organ transplantation rejection, (10) restenosis, (11) autologous bone marrow transplantation, (12) inflammatory sequelae of viral infections, (13) myocarditis, (14) inflammatory bowel disease including ulcerative colitis and Crohn's disease, (15) certain types of toxic and immune-based nephritis, (16) contact dermal hypersensitivity, (17) psoriasis, (18) tumor metastasis, (19) atherosclerosis, and (20) hepatitis.

The utilities of the present compounds in these diseases or disorders may be demonstrated in animal disease models that have been reported in the literature. The following are examples of such animal disease models:
i) experimental allergic encephalomyelitis, a model of neuronal demyelination resembling multiple sclerosis (for example, see T.

Yednock et al., “Prevention of experimental autoimmune encephalomyelitis by antibodies against. α4β1 integrin.” Nature, 356, 63 (1993) and E. Keszthelyi et al., “Evidence for a prolonged role of .α4 integrin throughout active experimental allergic encephalomyelitis.” Neurology, 47, 1053 (1996));
ii) bronchial hyperresponsiveness in sheep and guinea pigs as models for the various phases of asthma (for example, see W. M. Abraham et al., “α4-Integrins mediate antigen-induced late bronchial responses and prolonged airway hyperresponsiveness in sheep.” J. Clin. Invest. 93, 776 (1993) and A. A. Y. Milne and P. P. Piper, “Role of VLA-4 integrin in leucocyte recruitment and bronchial hyperresponsiveness in the guinea-pig.” Eur. J. Pharmacol., 282, 243 (1995));
iii) adjuvant-induced arthritis in rats as a model of inflammatory arthritis (see C. Barbadillo et al., “Anti-VLA4 mAb prevents adjuvant arthritis in Lewis rats.” Arthr. Rheuma. (Suppl.), 36 95 (1993) and D. Seiffge, “Protective effects of monoclonal antibody to VLA-4 on leukocyte adhesion and course of disease in adjuvant arthritis in rats.” J. Rheumatol., 23, 12 (1996));
iv) adoptive autoimmune diabetes in the NOD mouse (see J. L. Baron et al., “The pathogenesis of adoptive murine autoimmune diabetes requires an interaction between α4-integrins and vascular cell adhesion molecule-1.”, J. Clin. Invest., 93, 1700 (1994), A. Jakubowski et al., “Vascular cell adhesion molecule-Ig fusion protein selectively targets activated α4-integrin receptors in vivo: Inhibition of autoimmune diabetes in an adoptive transfer model in nonobese diabetic mice.” J. Immunol., 155, 938 (1995), and X. D. Yang et al., “Involvement of β7 integrin and mucosal addressin cell adhesion molecule-1 (MadCAM-1) in the development of diabetes in nonobese diabetic mice”, Diabetes, 46, 1542 (1997));
v) cardiac allograft survival in mice as a model of organ transplantation (see M. Isobe et al., “Effect of anti-VCAM-1 and anti-VLA-4 monoclonal antibodies on cardiac allograft survival and response to soluble antigens in mice.”, Tranplant. Proc., 26, 867 (1994) and S. Molossi et al., “Blockade of very late antigen-4 integrin binding to fibronectin with connecting segment-1 peptide reduces accelerated coronary arteripathy in rabbit cardiac allografts.” J. Clin Invest., 95, 2601 (1995));
vi) spontaneous chronic colitis in cotton-top tamarins which resembles human ulcerative colitis, a form of inflammatory bowel disease (see D. K. Podolsky et al., “Attenuation of colitis in the Cotton-top tamarin by anti-α4 integrin monoclonal antibody.”, J. Clin. Invest., 92, 372 (1993));
vii) contact hypersensitivity models as a model for skin allergic reactions (see T. A. Ferguson and T. S. Kupper, “Antigen-independent processes in antigen-specific immunity.”, J. Immunol., 150, 1172 (1993) and P. L. Chisholm et al., “Monoclonal antibodies to the integrin a-4 subunit inhibit the murine contact hypersensitivity response.” Eur. J. Immunol., 23, 682 (1993));
viii) acute nephrotoxic nephritis (see M. S. Mulligan et al., “Requirements for leukocyte adhesion molecules in nephrotoxic nephritis.”, J. Clin. Invest., 91, 577 (1993));
ix) tumor metastasis (for examples, see M. Edward, “Integrins and other adhesion molecules involved in melanocytic tumor progression.”, Curr. Opin. Oncol., 7, 185 (1995));
x) experimental autoimmune thyroiditis (see R. W. McMurray et al., “The role of α4 integrin and intercellular adhesion molecule-1 (ICAM-1) in murine experimental autoimmune thyroiditis.” Autoimmunity, 23, 9 (1996);
xi) ischemic tissue damage following arterial occlusion in rats (see F. Squadrito et al., “Leukocyte integrin very late antigen-4/vascular cell adhesion molecule-1 adhesion pathway in splanchnic artery occlusion shock.” Eur. J. Pharmacol., 318, 153 (1996; and
xii) inhibition of TH2 T-cell cytokine production including IL-4 and IL-5 by VLA-4 antibodies which would attenuate allergic responses (J. Clinical Investigation 100, 3083 (1997).
xiii) Shigematsu, T., Specian, R. D., Wolf, R. E., Grisham, M. B., and Granger, D. N. MADCAM mediates lymphocyte—endothelial cell adhesion in a murine model of chronic colitis. Am. J. Physiol. Gastrointest. Liver Physiol., 281: G1309-13015, 2001.
xiv) Picarella, D., Hurlbut, P., Torrman, J., Shi, X., Butcher, E., and Ringler, D. J. Monoclonal antibodies specific for β7 integrin and mucosal addressin cell adhesion molecule-1 (MAdCAM-1) reduce inflammation in the colon of scid mice reconstituted with CD45RB^{high} CD4⁺ T cells. J. Immuol., 158: 2099-2106. 1997.
xv) Hesterberg, P. E., Winsor-Hines, D., Briskin, M. J., et al., Rapid resolution of chronic colitis in the cotton-top tamarin with an antibody to a gut-homing integrin α4β7. Gastroenterology, 111:1373-1380, 1996.
xvi) Gordon, F. H., Lai, C. W. Y., Hamilton, M. I., Allison, M. C., Srivastava, E. D., Foutweather, M. G., Donoghue, S., Greenlee, C., Subhani, J., Amlot, P. L., and Pounder, R. E. A randomized placebo-controlled trial of a humanized monoclonal antibody to α4 integrin in active Crohn's disease. Gastroenterology, 121:268-274, 2001.
xvii) Ghosh, S., Goldin, E., Gordon, F. H., Malchow, H. A., Rask-madsen, J., Rutgeerts, P., Vyhnalek, P., Zadorova, Z, Palmer, T, and Donoghue, S. Natalizumab for active Crohn's disease. New Engl. J. Med., 348: 24-32, 2003.

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (a) other VLA-4 antagonists such as those described in U.S. Pat. No. 5,510,332, WO97/03094, WO97/02289, WO96/40781, WO96/22966, WO96/20216, WO96/01644, WO96/06108, WO95/15973 and WO96/31206; (b) steroids such as beclomethasone, methylprednisolone, betamethasone, prednisone, dexamethasone, and hydrocortisone; (c) immunosuppressants such as FK-506 type immunosuppressants; (d) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti-asthmatics such as b2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, praniukast, iralukast, pobilukast, SKB-106,203), leukotriene biosynthesis inhibitors (zileuton, BAY-1005); (f) non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); (g) cyclooxygenase-2 (COX-2) inhibitors such as celecoxib, rofecoxib, and parecoxib; (h) inhibitors of phosphodiesterase type IV (PDE-IV); (i) antagonists of the chemokine receptors, especially CCR-1, CCR-2, and CCR-3; (j) cholesterol lowering agents such as HMG-COA reductase inhibitors (lovastatin, simvastatin, pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; (k) anti-diabetic agents such as insulin, sulfonylureas, biguanides (mefformin), a-glucosidase inhibitors (acarbose) and glitazones (troglitazone, pioglitazone, englitazone, MCC-555, BRL49653 and the like); (1) agents that interfer with TNF such as antibodies to TNF (REMICADE®) or soluble TNF receptor (e.g. ENBREL®); (m) anticholinergic agents such as muscarinic antagonists (ipratropium nad tiatropium); (n) agents that slow gut motility such as opiate agonist (i.e. LOPERAMIDE®), serotonin receptor receptor anagonists (ALOSERTON, ODANSETRON, ect.) (O) other compounds such as 5-aminosalicylic acid and prodrugs thereof, antimetabolites such as azathioprine and 6-mercaptopurine, and cytotoxic cancer chemotherapeutic agents.

The weight ratio of the compound of the Formula I to the second active ingredient may be varied and will depend upon the effective dose of each ingredient. Generally, an effective dose of each will be used. Thus, for example, when a compound of the Formula I is combined with an NSAID the weight ratio of the compound of the Formula I to the NSAID will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

Accordingly, a compound of the present invention may be administered by any conventional route of administration including, but not limited to oral, nasal, pulmonary, sublingual, ocular, transdermal, rectal, vaginal and parenteral (i.e. subcutaneous, intramuscular, intradermal, intravenous etc.).

To prepare the pharmaceutical compositions of this invention, one or more compounds of Formula (I) or salt thereof as the active ingredient, is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing a pharmaceutical composition of the present invention in liquid dosage form for oral, topical and parenteral administration, any of the usual pharmaceutical media or excipients may be employed. Thus, for liquid dosage forms, such as suspensions (i.e. colloids, emulsions and dispersions) and solutions, suitable carriers and additives include but are not limited to pharmaceutically acceptable wetting agents, dispersants, flocculation agents, thickeners, pH control agents (i.e. buffers), osmotic agents, coloring agents, flavors, fragrances, preservatives (i.e. to control microbial growth, etc.) and a liquid vehicle may be employed. Not all of the components listed above will be required for each liquid dosage form.

In solid oral preparations such as, for example, dry powders for reconstitution or inhalation, granules, capsules, caplets, gelcaps, pills and tablets (each including immediate release, timed release and sustained release formulations), suitable carriers and additives include but are not limited to diluents, granulating agents, lubricants, binders, glidants, disintegrating agents and the like. Because of their ease of administration, tablets and capsules represent the most advantageous oral dosage unit form, in which case solid pharmaceutical carriers are obviously employed. If desired, tablets may be sugar coated, gelatin coated, film coated or enteric coated by standard techniques.

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg/kg to about 300 mg/kg (preferably from about 0.01 mg/kg to about 100 mg/kg; and, more preferably, from about 0.01 mg/kg to about 30 mg/kg) and may be given at a dosage of from about 0.01 mg/kg/day to about 300 mg/kg/day (preferably from about 0.01 mg/kg/day to about 100 mg/kg/day and more preferably from about 0.01 mg/kg/day to about 30 mg/kg/day). Preferably, the method for the treatment of integrin mediated disorders described in the present invention using any of the compounds as defined herein, the dosage form will contain a pharmaceutically acceptable carrier containing between from about 0.01 mg to about 100 mg; and, more preferably, from about 5 mg to about 50 mg of the compound, and may be constituted into any form suitable for the mode of administration selected. The dosages, however, may be varied depending upon the requirement of the subjects, the severity of the condition being treated and the compound being employed. The use of either daily administration or post-periodic dosing may be employed.

Preferably these compositions are in unit dosage forms from such as tablets, pills, capsules, dry powders for reconstitution or inhalation, granules, lozenges, sterile parenteral solutions or suspensions, metered aerosol or liquid sprays, drops, ampoules, autoinjector devices or suppositories for administration by oral, intranasal, sublingual, intraocular, transdermal, parenteral, rectal, vaginal, dry powder inhaler or other inhalation or insufflation means. Alternatively, the composition may be presented in a form suitable for once-weekly or once-monthly administration; for example, an insoluble salt of the active compound, such as the decanoate salt, may be adapted to provide a depot preparation for intramuscular injection.

For preparing solid pharmaceutical compositions such as tablets, the principal active ingredient is mixed with a pharmaceutical carrier, e.g. conventional tableting ingredients such as diluents, binders, adhesives, disintegrants, lubricants, antiadherents and gildants. Suitable diluents include, but are not limited to, starch (i.e. corn, wheat, or potato starch, which may be hydrolized), lactose (granulated, spray dried or anhydrous), sucrose, sucrose-based diluents (confectioner's sugar; sucrose plus about 7 to 10 weight percent invert sugar; sucrose plus about 3 weight percent modified dextrins; sucrose plus invert sugar, about 4 weight percent invert sugar, about 0.1 to 0.2 weight percent cornstarch and magnesium stearate), dextrose, inositol, mannitol, sorbitol, microcrystalline cellulose (i.e. AVICEL™ microcrystalline cellulose available from FMC Corp.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (i.e. alginic acid and salts thereof, magnesium aluminum silicate, hydroxyethylcellulose [i.e. TYLOSE™ available from Hoechst Celanese], polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like. Suitable lubricants and antiadherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like. Suitable gildants include, but are not limited to, talc, cornstarch, silica (i.e. CAB-O-SIL ™ silica available from Cabot, SYLOID™ silica available from W. R. Grace/Davison, and AEROSIL™ silica available from Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

Generally these carriers are mixed with the pharmaceutical active to form a solid preformulation composition containing a homogeneous mixture of the pharmaceutical active of the present invention, or a pharmaceutically acceptable salt thereof. Generally the preformulation will be formed by one of three common methods: (a) wet granulation, (b) dry granulation and (c) dry blending. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.1 mg to about 500 mg of the active ingredient of the present invention. The tablets or pills containing the novel compositions may also be formulated in multilayer tablets or pills to provide a sustained or provide dual-release products. For example, a dual release tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric materials such as shellac, cellulose acetate (i.e. cellulose acetate phthalate, cellulose acetate trimetilitate), polyvinyl acetate phthalate, hydroxypropyl methylcellulose phthalate, hydroxypropyl methylcellulose acetate succinate, methacrylate and ethylacrylate copolymers, methacrylate and methyl methacrylate copolymers and the like. Sustained release tablets may also be made by film coating or wet granulation using slightly soluble or insoluble substances in solution (which for a wet granulation acts as the binding agents) or low melting solids a molten form (which in a wet granulation may incorporate the active ingredient). These materials include natural and synthetic polymers waxes, hydrogenated oils, fatty acids and alcohols (i.e. beeswax, carnauba wax, cetyl alcohol, cetylstearyl alcohol, and the like), esters of fatty acids metallic soaps, and other acceptable materials that can be used to granulate, coat, entrap or otherwise limit the solubility of an active ingredient to achieve a prolonged or sustained release product.

The liquid forms in which the novel compositions of the present invention may be incorporated for administration orally or by injection include, but are not limited to aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable suspending agents for aqueous suspensions, include synthetic and natural gums such as, acacia, agar, alginate (i.e. propylene alginate, sodium alginate and the like), guar, karaya, locust bean, pectin, tragacanth, and xanthan gum, cellulosics such as sodium carboxymethylcellulose, methylcellulose, hydroxymethylcellulose, hydroxyethylcellulose, hydroxypropyl cellulose and hydroxypropyl methylcellulose, and combinations thereof, synthetic polymers such as polyvinyl pyrrolidone, carbomer (i.e. carboxypolymethylene), and polyethylene glycol; clays such as bentonite, hectorite, attapulgite or sepiolite; and other pharmaceutically acceptable suspending agents such as lecithin, gelatin or the like. Suitable surfactants include but are not limited to sodium docusate, sodium lauryl sulfate, polysorbate, octoxynol-9, nonoxynol-10, polysorbate 20, polysorbate 40, polysorbate 60, polysorbate 80, polyoxamer 188, polyoxamer 235 and combinations thereof. Suitable deflocculating or dispersing agent include pharmaceutical grade lecithins. Suitable flocculating agent include but are not limited to simple neutral electrolytes (i.e. sodium chloride, potassium, chloride, and the like), highly charged insoluble polymers and polyelectrolyte species, water soluble divalent or trivalent ions (i.e. calcium salts, alums or sulfates, citrates and phosphates (which can be used jointly in formulations as pH buffers and flocculating agents). Suitable preservatives include but are not limited to parabens (i.e. methyl, ethyl, n-propyl and n-butyl), sorbic acid, thimerosal, quaternary ammonium salts, benzyl alcohol, benzoic acid, chlorhexidine gluconate, phenylethanol and the like. There are many liquid vehicles that may be used in liquid pharmaceutical dosage forms, however, the liquid vehicle that is used in a particular dosage form must be compatible with the suspending agent(s). For example, nonpolar liquid vehicles such as fatty esters and oils liquid vehicles are best used with suspending agents such as low HLB (Hydrophile-Lipophile Balance) surfactants, stearalkonium hectorite, water insoluble resins, water insoluble film forming polymers and the like. Conversely, polar liquids such as water, alcohols, polyols and glycols are best used with suspending agents such as higher HLB surfactants, clays silicates, gums, water soluble cellulosics, water soluble polymers and the like. For parenteral administration, sterile suspensions and solutions are desired. Liquid forms useful for parenteral administration include sterile solutions, emulsions and suspensions. Isotonic preparations which generally contain suitable preservatives are employed when intravenous administration is desired.

Furthermore, compounds of the present invention can be administered in an intranasal dosage form via topical use of suitable intranasal vehicles or via transdermal skin patches, the composition of which are well known to those of ordinary skill in that art. To be administered in the form of a transdermal delivery system, the administration of a therapeutic dose will, of course, be continuous rather than intermittent throughout the dosage regimen.

Compounds of the present invention can also be administered in the form of liposome delivery systems, such as small unilamellar vesicles, large unilamellar vesicles, multilamellar vesicles and the like. Liposomes can be formed from a variety of phospholipids, such as cholesterol, stearylamine, phosphatidylcholines and the like.

Compounds of the present invention may also be delivered by the use of monoclonal antibodies as individual carriers to which the compound molecules are coupled. The compounds of the present invention may also be coupled with soluble polymers as targetable drug carriers. Such polymers can include, but are not limited to polyvinylpyrrolidone, pyran copolymer, polyhydroxypropylmethacrylamidephenol, polyhydroxy-ethylaspartamidephenol, or polyethyl eneoxidepolylysine substituted with palmitoyl residue. Furthermore, the compounds of the present invention may be coupled to a class of biodegradable polymers useful in achieving controlled release of a drug, for example, to homopolymers and copolymers (which means polymers containing two or more chemically distinguishable repeating units) of lactide (which includes lactic acid d-, l- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, γ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, polyorthoesters, polyacetals, polydihydropyrans, polycyanoacrylates and cross-linked or amphipathic block copolymers of hydrogels and blends thereof.

Compounds of this invention may be administered in any of the foregoing compositions and dosage regimens or by means of those compositions and dosage regimens established in the art whenever treatment of integrin mediated disorders is required for a subject in need thereof.

The daily dose of a pharmaceutical composition of the present invention may be varied over a wide range from about 0.7 mg to about 21,000 mg per adult human per day; preferably, the dose will be in the range of from about 0.7 mg to about 7000 mg per adult human per day; most preferably the dose will be in the range of from about 0.7 mg to about 2100 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated. An effective amount of the drug is ordinarily supplied at a dosage level of from about 0.01 mg/kg to about 300 mg/kg of body weight per day. Advantageously, a compound of the present invention may be administered in a single daily dose or the total daily dosage may be administered in divided doses of two, three or four times daily.

Optimal dosages to be administered may be readily determined by those skilled in the art, and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level.

Representative I UPAC names for the compounds of the present invention were derived using the ACD/LABS SOFTWARE™ Index Name Pro Version 4.5 nomenclature software program provided by Advanced Chemistry Development, Inc., Toronto, Ontario, Canada.

Abbreviations used in the instant specification, particularly the Schemes and Examples, are as follows:
Boc=tert-butoxycarbonyl
BOC-ON=2-(tert-butoxycarbonyloxyimino)-2-phenylacetonitrile
BOP-Cl=Bis-(2-oxo-3-oxazolidinyl)phosphinic chloride
BuLi=n-butyllithium
t-BuOH=tert-butanol
CDI=1,1′-carbonyldiimidazole
Cpd or Cmpd=compound
d=day/days
DCM=dichloromethane
DIPEA=diisopropylethylamine
EDC=1-(3-Dimethylaminopropyl)-3-ethylcarbodiimide hydrochloride
EtOAc=ethyl acetate
EtOH=ethanol
h=hour/hours
HOBt/HOBT=hydroxybenzotriazole
LDA=lithium diisopropyamide
M=molar
MeCN=acetonitrile
MeOH=methanol
min=minutes
NMM=N-methylmorpholine
NT=not tested
rt/RT=room temperature
THF=tetrahydrofuran
TFA=trifluoroacetic acid
TsOH=para-toluenesulfonic acid

### General Synthetic Methods

Representative compounds of the present invention can be synthesized in accordance with the general synthetic methods described below and are illustrated more particularly in the schemes that follow. Since the schemes are an illustration, the invention should not be construed as being limited by the chemical reactions and conditions expressed. The preparation of the various starting materials used in the schemes is well within the skill of persons versed in the art.

The following schemes describe general synthetic methods whereby intermediate and target compounds of the present invention may be prepared. Additional representative compounds and stereoisomers, racemic mixtures, diasteromers and enantiomers thereof can be synthesized using the intermediates prepared in accordance to the general schemes and other materials, compounds and reagents known to those skilled in the art. All such compounds, stereoisomers, racemic mixtures, diasteromers and enantiomers thereof are intended to be encompassed within the scope of the present invention.

Scheme A describes a general method for the synthesis of optionally substituted pyridazinone intermediates which may be further reacted to give compounds of the present invention. R³ substituents may be introduced into pyridazinones through the cyclization of Compound A1 with a hydrazine precursor (Compound A2), to produce Compound A3. Alternatively, R³ may be introduced by alkylation, as shown in Scheme AA, provided that R³≠H in R³—X.

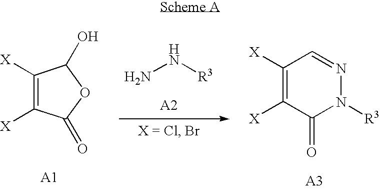

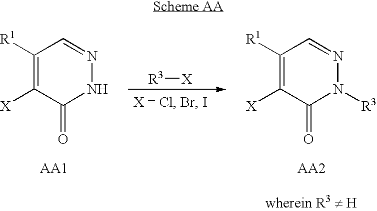

As shown in Scheme B, R¹ may be introduced into 4-halopyridazinones by selective displacement of the 5-X substituent of Compound A3 with a desired functional group. For example, selective arylation of Compound A3 with an aryl boronic acid and a palladium catalyst provides Compound B1. Compound A3 may also be reacted at the 5-position with an alcohol or amine to give Compound AA2 wherein R¹ is alkoxy or amino as defined herein.

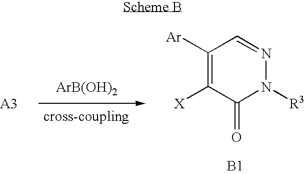

Scheme C illustrates another route to substituted pyridazinones by displacing a 5-methoxy group. Compound C1 may be treated with an alcohol and base to form Compound C2 wherein R¹ is a new alkoxy substituent as defined within the scope of this invention.

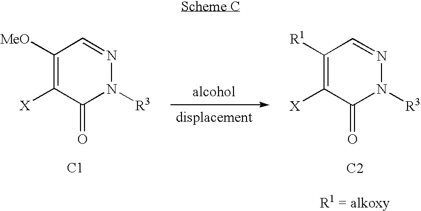

Scheme D describes a general method for preparing compounds of the present invention. Compound D1 may be reacted with a 4-halogen substituted pyridazinone (D2) in the presence of a palladium catalyst and an appropriate base such as sodium carbonate to afford Compound D3. The carboxy group of Compound D3 may be protected as its methyl ester, Compound D4, using conventional chemistry. Compound D4 may then be acylated with an acid chloride to afford Compound D5. Alternatively, Compound D4 may be acylated through a coupling reaction with a carboxylic acid in the presence of an appropriate coupling agent, base, and solvent. One example of an appropriate set of coupling reagents is using EDC and HOBt as coupling agents with triethylamine in dichloromethane. The substituent Z may be further elaborated using chemistry known to those skilled in the art. Compound D6 may be obtained upon deprotection of Compound D5.

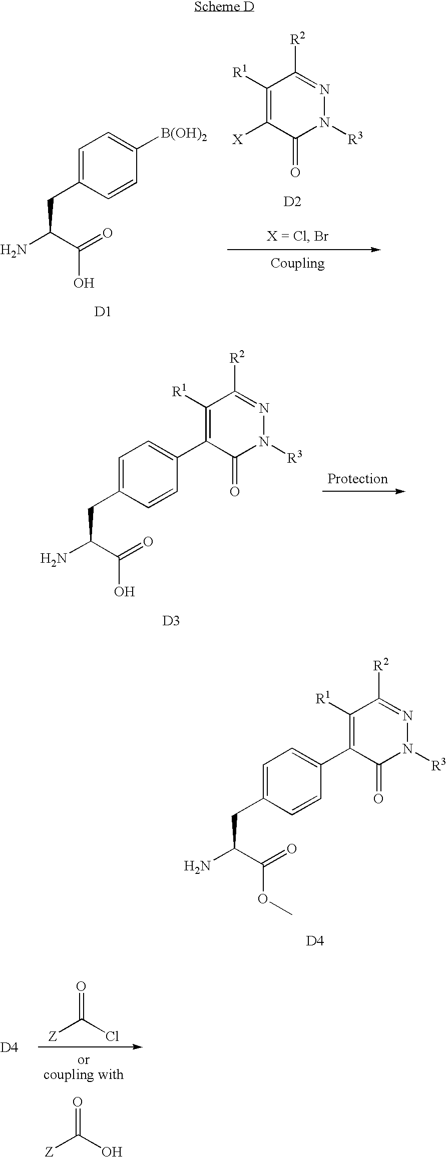

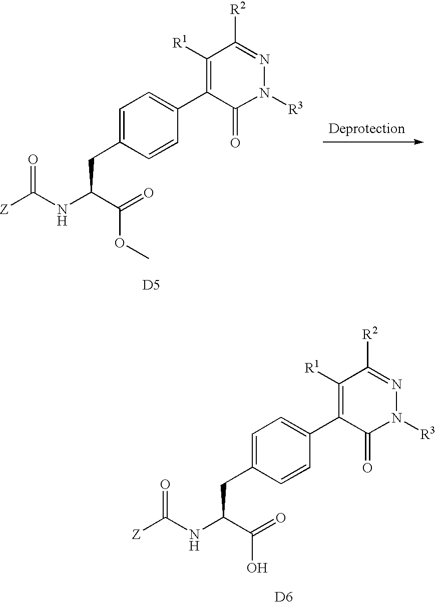

Those skilled in the art will recognize that the construction of compounds of Formula D6 may be completed by manipulation of the reaction sequence of Scheme D. As shown in Scheme E, Compound D1 may be acylated with an acid chloride to yield Compound E1, which may then be coupled to Compound D2 in the presence of a palladium catalyst to give Compound D6. 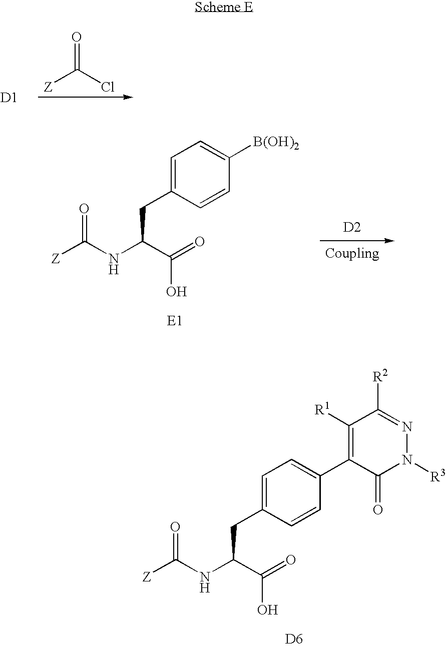
Alternatively, Compound D3 may be directly acylated by reaction with an acid chloride to provide Compound D6.

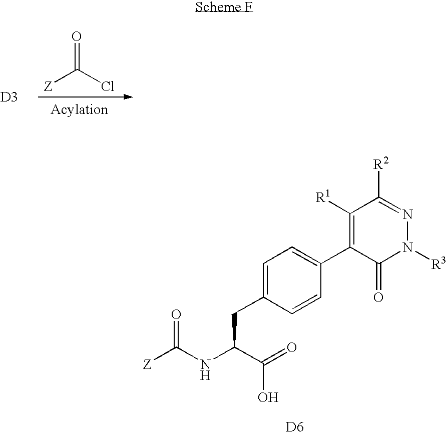

Compounds of the present invention wherein W is sulfur can be prepared by treatment of Compound D6 with Lawesson's Reagent as illustrated in Scheme G.

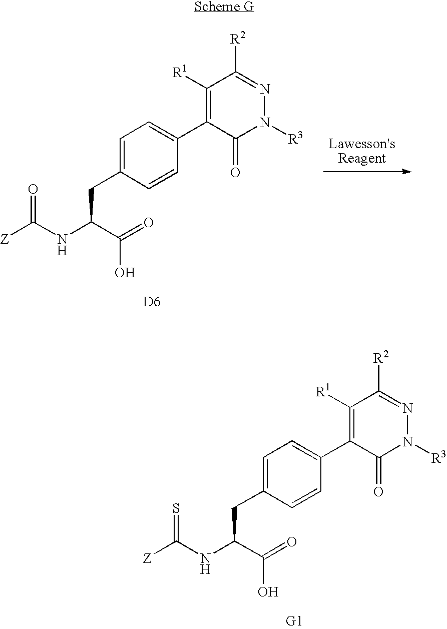

Scheme H describes the preparation of compounds of the present invention wherein R¹ is heteroaryl. Compound H1 wherein R¹ is methoxy, may be reacted with an NH-containing heteroaryl compound under basic conditions in a microwave reactor to afford Compound H2.

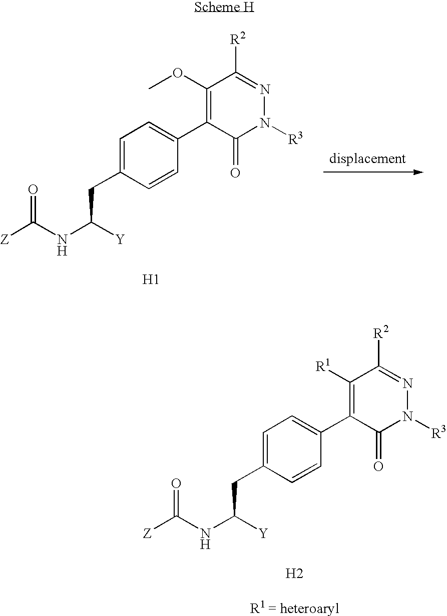

Compound D4 may be acylated with CDI and the resultant carbamoyl imidazole activated by reaction with methyl iodide. Upon methylation, this intermediate may be treated with an alkoxide to form Compound J1. Basic hydrolysis of Compound J1 provides Compound J2.

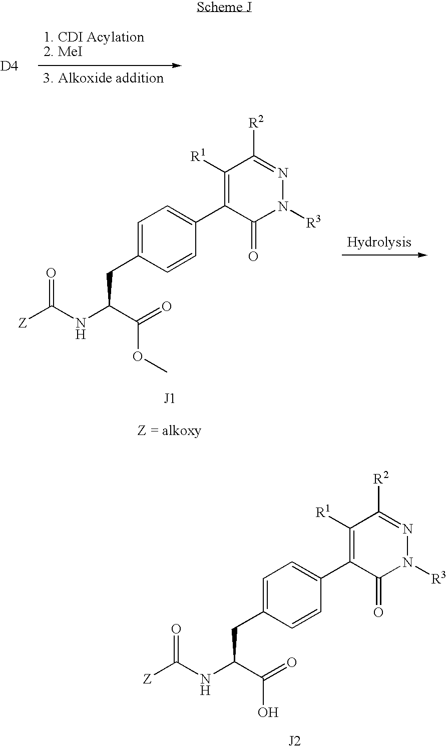

Carbamates of the present invention (wherein Z is an alkoxy substituent) may be synthesized by alternative routes. For example, the amino group of Compound D4 may be treated with a chloroformate or a dialkyldicarbonate to afford a carbamate intermediate, which may be hydrolyzed under basic conditions to yield Compound J2.

Scheme K describes the preparation of compounds of the present invention wherein Y is tetrazole. Boc-protected compound K1 may be synthesized according to the literature (Samanen, et al. J. Med. Chem. 1988, 31, 510-516), and then may be coupled to compound D2 as described above to afford Compound K2. Compound K2 may be treated with ammonium bicarbonate and di-tert-butyl-dicarbonate to provide the primary amide, Compound K3. Compound K3 may be reacted with cyanuric chloride to give Compound K4, which may then be reacted with sodium azide in the presence of zinc bromide to yield Compound K5. Acylation of Compound K5 by a method described in Scheme D yields Compound K6.

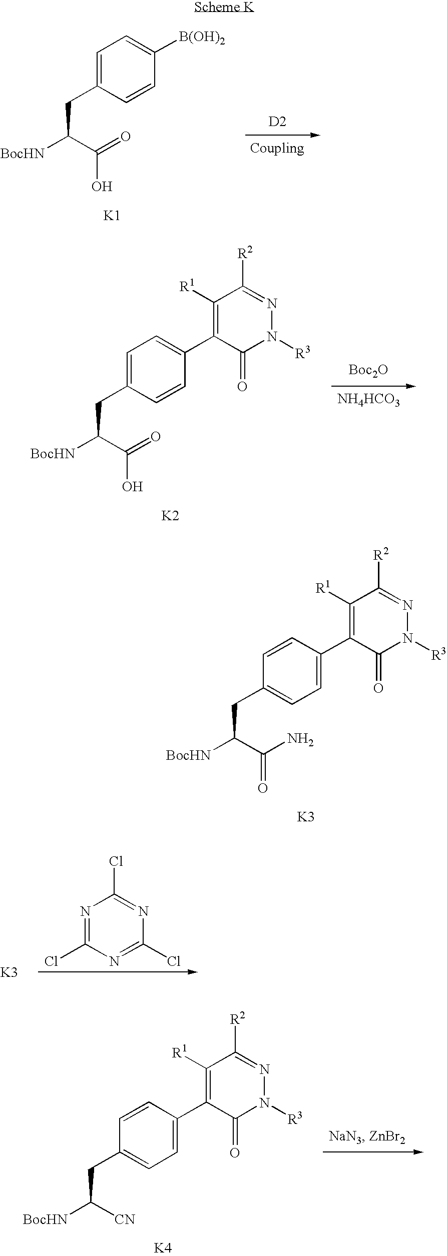

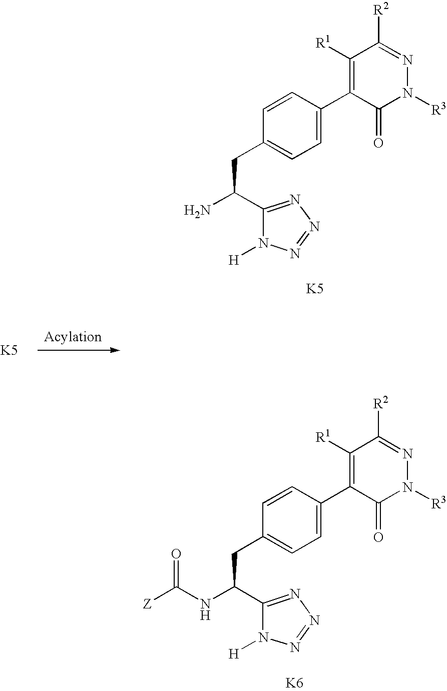

Scheme L illustrates the preparation of compounds of the present invention wherein Y is —C(═O)NHSO₂(C₁₋₄)alkyl. Compound D6 may be coupled with alkylsulfonamides in the presence of an appropriate coupling agent, base and solvent to yield Compound L1. Compounds of the present invention were prepared in the presence of EDC and DMAP in DCM.

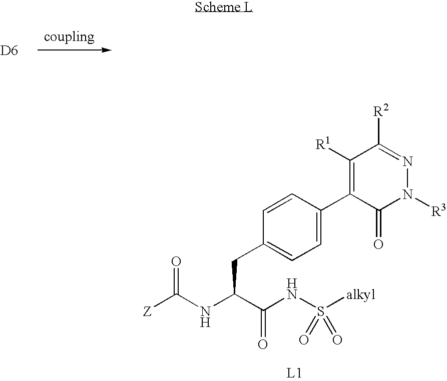

Scheme M describes the preparation of compounds of the present invention wherein Y is hydroxymethyl. Treatment of Compound D5 with an appropriate hydride source, preferably a metalloborohydride, affords the corresponding alcohol (Compound M1).

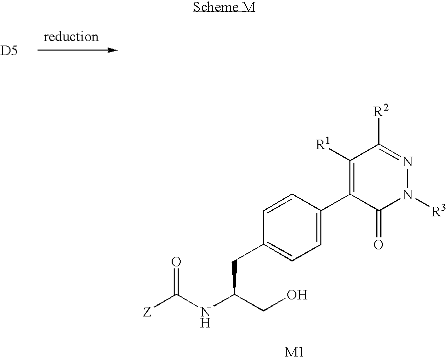

As shown in Scheme N, R¹ and R² can be taken together to form a heterocycle. Compound N1 may be reacted with ethylene glycol under basic conditions to afford Compound N2, which may be coupled with an aryl boronic acid such as E1 and a palladium catalyst to afford compounds of the present invention.

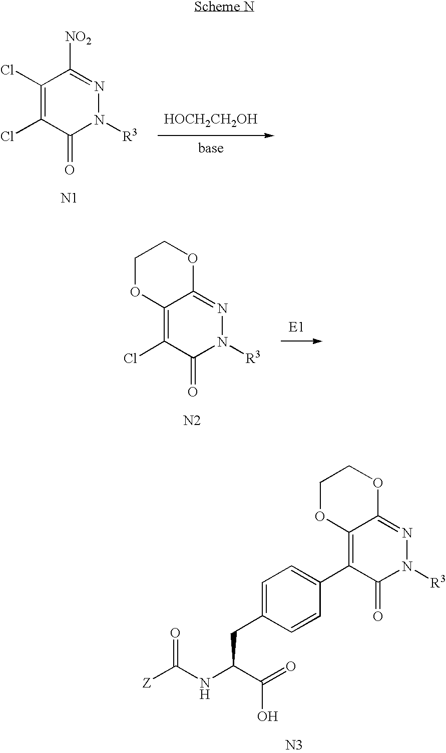

Scheme P further illustrates the preparation of compounds of the present invention wherein R¹ and R² form a heterocyclic ring. Compound N1 may be reacted with ethanolamine with microwave irradiation to give Compound P1. Compound P1 may be coupled with a boronic acid such as Compound E1 using a palladium catalyst to provide Compound P2.

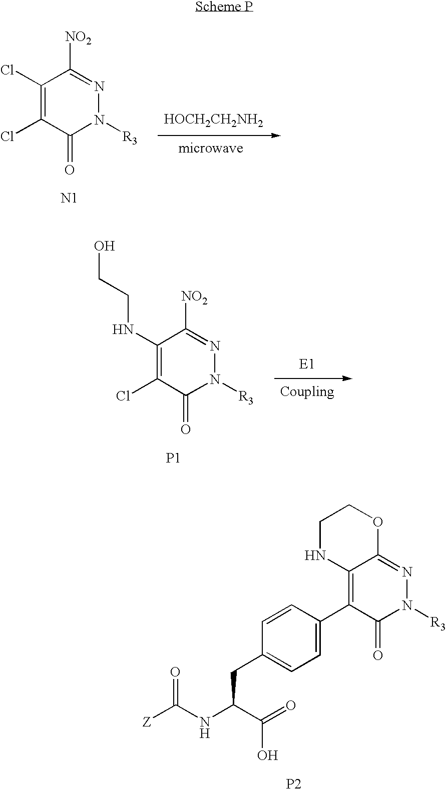

Scheme Q describes the preparation of compounds of the present invention wherein Y, the amino group, and the atoms to which they are attached are covalently bound to form a ring. Compound D5 may be reacted with paraformaldehyde and para-toluenesulfonic acid to form Compound Q1.

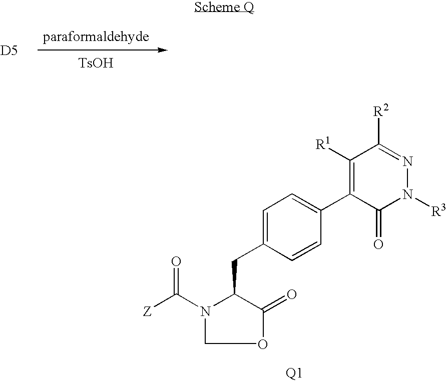

Ester prodrugs of compounds of the present invention may be prepared from Compound D6 by methods know to those skilled in the art. For instance, as illustrated in Scheme R, Compound D6 may be reacted with an alcohol using an appropriate coupling agent such as bis(2-oxo-3-oxazolidinyl)phosphinic chloride, affording Compound R1 wherein Y is an optionally substituted —C(═O)C₁₋₆alkoxy as defined herein. Alternatively, Compound D6 may be converted to an acid chloride intermediate using conventional chemistry known to one skilled in the art, and the acid chloride may then be treated with an alcohol to yield Compound R1.

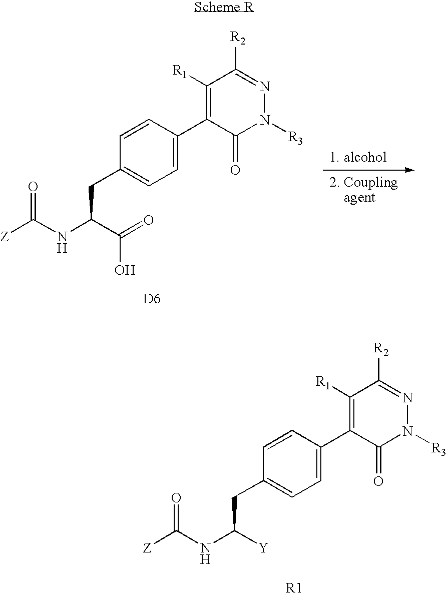

### Specific Synthetic Methods

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The instant compounds may also be used as intermediates in subsequent examples to produce additional compounds of the present invention. No attempt has been made to optimize the yields obtained in any of the reactions. One skilled in the art would know how to increase such yields through routine variations in reaction times, temperatures, solvents and/or reagents.

Reagents were purchased from commercial sources. Nuclear magnetic resonance (NMR) spectra for hydrogen atoms were measured in the indicated solvent with (TMS) as the internal standard on a Bruker AM-360 (360 MHz) spectrometer. The values are expressed in parts per million down field from TMS. The mass spectra (MS) were determined on a Micromass Platform LC spectrometer or an Agilent LC spectrometer using electrospray techniques. Microwave accelerated reactions were performed using either a CEM Discover or a Personal Chemistry Smith Synthesizer microwave instrument. Stereoisomeric compounds may be characterized as racemic mixtures or as separate diastereomers and enantiomers thereof using X-ray crystallography and other methods known to one skilled in the art. Unless otherwise noted, the materials used in the examples were obtained from readily available commercial suppliers or synthesized by standard methods known to one skilled in the art of chemical synthesis. The substituent groups, which vary between examples, are hydrogen unless otherwise noted.

### EXAMPLE 1

### (S)-2-Amino-3-[4-(5-methoxy-2-methyl-3-oxo-2,3-di hydro-pyridazin-4-yl)-phenyl]-propionic acid, Cpd 17

A 10 mL vial containing a magnetic stir bar was charged with Compound 1a (4-borono-L-phenylalanine) (110 mg, 0.50 mmol), 4-chloro-5-methoxy-2-methyl-2H-pyridazin-3-one (Compound 1b) (79 mg, 0.45 mmol), dichlorobis(triphenylphosphine)palladium (II) (18 mg, 0.025 mmol), 1.0 M sodium carbonate (1.0 mL, 1.0 mmol) and acetonitrile (1.0 mL). The vial was sealed and the mixture was heated under microwave irradiation at 150° C. for 10 min. The crude mixture, upon acidification with TFA and removal of the solvents, was purified by reverse phase HPLC (0.1% TFA H₂O/MeCN, 0-20% gradient) to yield Compound 1c as a white solid (TFA salt, 125 mg). ¹H NMR (CD₃OD) δ: 8.20 (s, 1H), 7.42 (d, 2H), 7.35 (d, 2H), 4.24, (dd, 1H), 3.92 (s, 3H), 3.80 (s, 3H), 3.37 (dd, 1H), 3.14 (dd, 1H). MS m/z: M+1=304.

Compound 1c (TFA salt, 0.20 g, 0.48 mmol) was dissolved in MeOH (8 mL) and heated in the presence of SOCl₂ (0.2 mL) at 80° C. for 2 h. The solution was concentrated, and the resulting solid was treated with saturated NaHCO₃ (aq) and extracted with CH₂Cl₂ (3×2 mL). The organic phase was dried (MgSO₄), filtered, and concentrated to a clear gum to give Compound 1d (0.10 g). ¹H NMR (CDCl₃) δ: 7.88 (s, 1H), 7.48 (d, 2H), 7.24 (d, 2H), 3.90 (s, 3H), 3.80 (s, 3H), 3.80 (s, 3H), 3.77 (dd, 1H), 3.73 (s, 3H), 3.15 (dd, 1H), 2.86 (dd, 1H). MS m/z: M+1=318.

To Compound 1d (0.33 g, 1.0 mmol) in CH₂Cl₂ (10 mL) was added Et₃N (0.35 mL, 2.5 mmol) and Compound 1e (2,6-dichlorobenzoyl chloride) (0.29 mL, 2.0 mmol). The reaction was quenched with saturated NaHCO₃ (aq) after 1 h, and concentrated to a residue. The crude mixture was purified by reverse phase HPLC (0.1% TFA H₂O/MeCN, 25-45% gradient) to yield Compound 1f as a white solid (0.40 g). ¹H NMR (CDCl₃) δ: 7.88 (s, 1H), 7.46 (d, 2H), 7.25-7.30 (m, 5H), 6.35 (br, 1H), 5.25 (m, 1H), 3.89 (s, 3H), 3.80 (s, 3H), 3.80 (s, 3H), 3.77 (dd, 1H), 3.73 (s, 3H), 3.30 (m, 2H). MS m/z: M+1=490.

Compound 1f (0.21 g, 0.43 mmol) was treated with 1N LiOH (1.0 mL) in MeOH (5 mL) at rt for 4 h. The slurry, after removal of MeOH, was dissolved in water (4 mL) and washed with CH₂Cl₂ (2×2 mL) before being acidified with aqueous HCl. The precipitate was collected by filtration, washed with water (3×), and dried in a vacuum oven (50° C.) to yield Compound 17 as a white solid (0.18 g). ¹H NMR (CD₃OD) δ: 8.18 (s, 1H), 7.38-7.33 (m, 7H), 4.99 (dd, 1H), 3.94 (s, 3H), 3.78 (s, 3H), 3.33 (dd, 1H), 3.08 (dd, 1H). MS m/z: M+1=476.

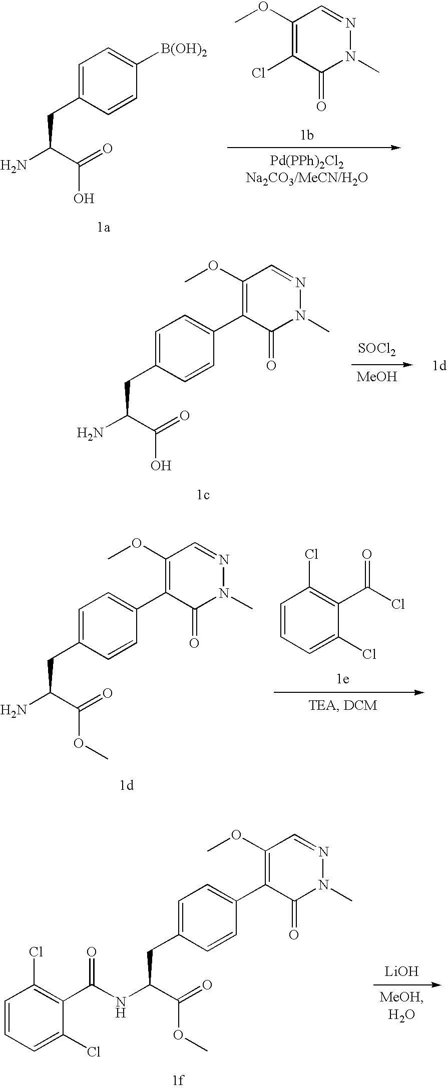

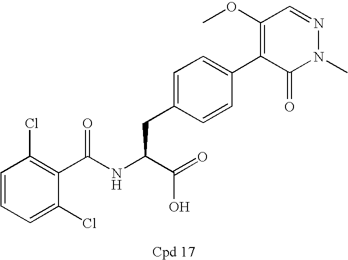

Other compounds of the present invention may be prepared by those skilled in the art by varying the starting materials, reagent(s) and conditions used. Using the procedure of Example 1, the following compounds were prepared:

| Cpd | MS (M + H) | Cpd | MS (M + H) |
|---|---|---|---|
| 45 | 385 | 55 | 477 |
| 76 | 443 | | |

### EXAMPLE 1-1

### (S)-2-(2,6-Dichloro-benzoylamino)-3-[4-(5-hydroxy-2-methyl-3-oxo-2,3-dihydro-pyridazin-4-yl)-phenyl]-propionic acid, Cpd 95

Compound 17 (40 mg, 0.084 mmol) was heated in HBr (40%, 0.2 mL) and AcOH (0.2 mL) at 130° C. for 20 min under microwave irradiation. The reaction mixture was concentrated to a residue and purified by HPLC to give Compound 95 as a white solid (9 mg). ¹H NMR (CD₃OD) δ: 7.77 (s, 1H), 7.35-7.44 (m, 7H), 4.98 (dd, 1H), 3.73 (s, 3H), 3.30 (dd, 1H), 3.12 (dd, 1H). MS m/z: M+1=462.

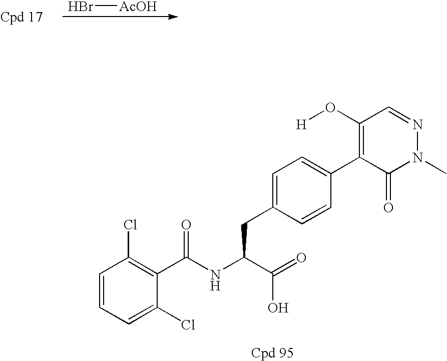

Other compounds of the present invention may be prepared by those skilled in the art by varying the starting materials, reagent(s) and conditions used. Using the procedure of Example 1-1, the following compounds were prepared:

| Cpd | MS (M + H) |
|---|---|
| 62 | 463 |
| 168 | 448 |

### EXAMPLE 2

### (S)-3-[4-(5-Methoxy-2-methyl-3-oxo-2,3-dihydro-pyridazin-4-yl)-phenyl]-2-[(2,2,3,3-tetramethyl-cyclopropanecarbonyl)-amino]-propionic acid, Cpd 136

A mixture of Compound 1d (32 mg, 0.10 mmol), 2,2,3,3-tetramethyl-cyclopropanecarboxylic acid (Compound 2a) (17 mg, 0.12 mmol), EDC (23 mg, 0.12 mmol), HOBt (16 mg, 0.12 mmol) and Et₃N (0.17 μL, 0.12 mmol) in CH₂Cl₂ (2 mL) was stirred at rt for 16 h. The reaction mixture was washed with water, then with saturated NaHCO₃ (aq) and concentrated under reduced pressure to dryness. The residue was hydrolyzed in MeOH (1 mL) containing 1 M LiOH (0.3 mL) for 4 h. Acidification followed by reverse phase HPLC purification (0.1% TFA H₂O/MeCN, 25-45% gradient) yielded Compound 136 as a white solid (23 mg). ¹H NMR (CD₃OD) δ: 8.19 (s, 1H), 7.38 (d, 2H), 7.28 (d, 2H), 4.66 (dd, 1H), 3.94 (s, 3H), 3.79 (s, 3H), 3.19 (dd, 1H), 2.99 (dd, 1H), 1.19 (s, 3H), 1.13(s, 3H), 1.11(s, 3H), 1.08 (s, 3H). MS m/z: M+1=428.

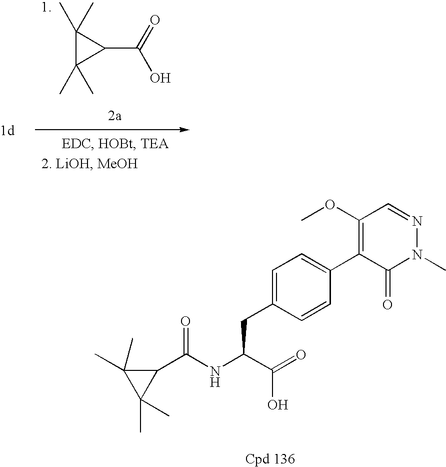

Other compounds of the present invention may be prepared by those skilled in the art by varying the starting materials, reagent(s) and conditions used. Using the procedure of Example 2, the following compounds were prepared:

| Cpd | MS (M + H) | Cpd | MS (M + H) |
|---|---|---|---|
| 124 | 388 | 169 | 477 |
| 127 | 404 | 170 | 438 |
| 129 | 404 | 173 | 389 |
| 130 | 506 | 177 | 487 |
| 132 | 492 (M − H) | 185 | 390 |
| 133 | 701 | 191 | 425 |
| 137 | 452 | 186 | 531 |
| 138 | 452 | 187 | 448 |
| 143 | 402 | 188 | 487 |
| 144 | 427 | 193 | 439 |
| 145 | 533 | 195 | 501 |
| 147 | 413 | 177 | 487 |
| 154 | 604 | 200 | 448 |
| 157 | 473 (M − H) | 202 | 515 |
| 161 | 535 | 205 | 398 |
| 163 | 442 | | |
| 167 | 604 | | |

### EXAMPLE 3

### (S)-2-(2-Chloro-4-methanesulfonyl-benzoylamino)-3-[4-(5-methoxy-2-methyl-3-oxo-2,3-dihydro-pyridazin-4-yl)-phenyl]-propionic acid, Cpd 31

To crude Compound 1c in Na₂CO₃/H₂O/CH₃CN (0.50 mmol, as described in EXAMPLE 1) was added 2-chloro-4-methanesulfonyl-benzoyl chloride (0.36 g, 1.4 mmol) in acetonitrile (1 mL) and the mixture was stirred at 50° C. for 15 min. Acidification followed by reverse phase HPLC purification (0.1% TFA H₂O/MeCN, 20-40% gradient) gave Compound 31 as a white solid (81 mg). ¹H NMR (CD₃OD) δ: 8.18 (s, 1H), 7.98 (d, 1H), 7.95 (d, 1H), 7.47 (d, 1H), 7.41-7.33 (m, 4H), 4.96 (dd, 1H), 3.94 (s, 3H), 3.78 (s, 3H), 3.40 (dd, 1H), 3.14 (s, 3H), 3.07 (dd, 1H). MS m/z: M+1=520.

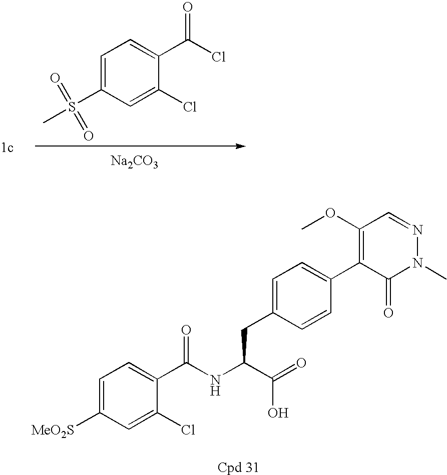

### EXAMPLE 4

### (S)-2-(2,6-Dichloro-thiobenzoylamino)-3-[4-(5-methoxy-2-methyl-3-oxo-2,3-dihydro-pyridazin-4-yl)-phenyl]-propionic acid, Cpd 140

Lawesson's reagent (2,4-bis(4-methoxyphenyl)-1,3-dithia-2,4-diphosphetane-2,4-disulfide, 83.9 mg, 208 μmol) was added to a suspension of Compound 17 (198 mg, 0.415 mmol) in toluene (2 mL). The suspension was heated to reflux for 15 min, resulting in formation of a yellow solution. The solution was allowed to cool to 23° C. and was concentrated. The residue was suspended in acetonitrile and was acidified by addition of TFA. The resulting solution was filtered and was purified by reverse-phase HPLC (YMC Pack ODS-A column, gradient elution from 20-50% acetonitrile-water, both containing 0.1% TFA). The column eluant was lyophilized, yielding Compound 140 as a white powder (43.7 mg). (MS ES+) m/z 514 (M+Na)⁺.

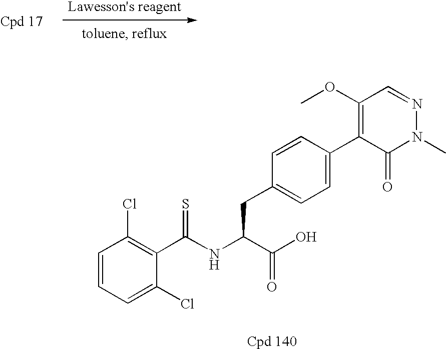

### EXAMPLE 5

### (S)-3-[4-(5-Cyclopropyl methoxy-2-methyl-3-oxo-2,3-dihydro-pyridazin-4-yl)-phenyl]-2-(2,6-dichloro-benzoylamino)-propionic acid, Cpd 86

Sodium (25 mg, 1.09 mmol) was added to cyclopropylmethyl alcohol (Compound 5a, 1.0 mL) in a pressure tube. The suspension was stirred at 23° C. until the sodium had dissolved (45 min). Compound 17 (100 mg, 210 μmol) was added and the reaction vessel was sealed and was placed in a 85° C. oil bath. The mixture was heated for 1 h, then was allowed to cool to 23° C. and was concentrated. To the residue was added acetonitrile and the resulting mixture was acidified by addition of TFA and was filtered. The filtrate was purified by reverse-phase HPLC (YMC Pack ODS-A column, gradient elution from 25-50% acetonitrile-water, both containing 0.1% TFA) to yield Compound 86 as a white powder (87.6 mg). (MS ES+) m/z 516.1 (M+H).

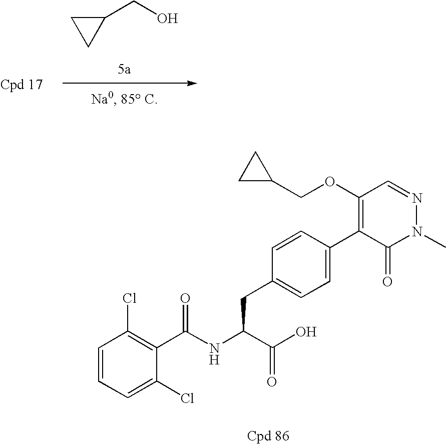

Other compounds of the present invention may be prepared by those skilled in the art by varying the starting materials, reagent(s) and conditions used. Using the procedure of Example 5, the following compounds were prepared:

| Cpd | MS (M + H⁺) |
|---|---|
| 7 | 550 |
| 8 | 506 |
| 12 | 545 |
| 25 | 553 |
| 41 | 544 |
| 52 | 552 |
| 60 | 552 (M + Na) |
| 74 | 504 |
| 150 | 533 |

### EXAMPLE 6

### (S)-2-Benzyloxycarbonylamino-3-[4-(5-methoxy-2-methyl-3-oxo-2,3-dihydro-pyridazin-4-yl)-phenyl]-propionic acid, Cpd 172

Compound 6a was prepared from Compound 1a by the method of Samanen, et al. J. Med. Chem. 1988, 31, 510-516.

To a mixture of Compound 6a (12.86 g, 41.6 mmol), Compound 6b (Cho, S.-D.; Choi, W.-Y.; Yoon, Y.-J. J. Heterocycl. Chem. 1996, 33, 1579-1582) (10.02 g, 45.8 mmol) and trans-dichloro(bistriphenylphosphine)palladium (II) (1.46 g, 2.08 mmol) were sequentially added a solution of Na₂CO₃ (aq)(2 M, 84 mL, 168 mmol) and CH₃CN (84 mL). The resulting suspension was heated at reflux under N₂ for 1 h, then was allowed to cool to 23° C. The mixture was partially concentrated to remove volatile solvent. The resulting mixture was diluted with one-quarter saturated NaHCO₃ (aq) (200 mL) and was washed with Et₂O (200 mL). The organic phase was back-extracted with one-quarter saturated NaHCO₃ (aq) (200 mL). The combined aqueous extracts were cooled to 0° C. and were acidified to pH 2 by addition of 2 N aqueous HCl. The precipitated solid was collected by vacuum filtration, affording crude Compound 6c (15.18 g). A sample of purified Compound 6c was obtained by reverse-phase HPLC (YMC Pack ODS-A column, gradient elution from 23 to 43% CH₃CN-water, both containing 0.1% TFA). (MS ES+) m/z 426 (M+Na)⁺.

Trimethylsilyldiazomethane (2 M solution in hexanes, 28.0 mL, 56.0 mmol) was added to a solution of crude Compound 6c (15.18 g) in benzene:MeOH (7:2, 135 mL). The resulting mixture was stirred at 23° C. for 17 h. The mixture was concentrated and the residue was purified by column chromatography (gradient elution from 50 to 90% EtOAc-hexanes), yielding Compound 6d as a white foam (7.83 g). (TOF MS ES+) m/z 440 (M+Na)⁺.

TFA (819 μL, 10.6 mmol) was added to a solution of Compound 6d (443 mg, 1.06 mmol) in CH₂Cl₂. The resulting solution was stirred at 23° C. for 3 h. The solution was concentrated and the residue was purified by flash column chromatography (silica gel, gradient elution from 2 to 10% MeOH—CH₂Cl₂) to yield a white foam (539 mg). (TOF MS ES+) m/z 318 (M+H)⁺. To a solution of the foam in CH₂Cl₂:THF (5:1, 6 mL) was added 1,1′-carbonyldiimidazole (259 mg, 1.59 mmol). The resulting solution was stirred at 23° C. for 1 h. The mixture was concentrated and the residue was purified by column chromatography (silica gel, gradient elution from 2 to 10% MeOH—CH₂Cl₂). Compound 6e was obtained as a white solid (355 mg). (TOF MS ES+) m/z 412 (M+H)⁺.

Methyl iodide (50.5 μL, 811 μmol) was added to a solution of Compound 6e (83.4 mg, 203 [mol). The resulting mixture was stirred at 23° C. for 16 h, then was concentrated, yielding a light yellow oil. To a solution of the residue in THF:DMF (1:1, 1 mL) was added benzyl alcohol (Compound 6f, 21.7 μL, 203 μmol) followed by sodium hydride (60% dispersion in mineral oil, 8.9 mg, 223 μmol). The resulting yellow solution was stirred at 23° C. for 4 h. An aqueous solution of LiOH (2 N, 1 mL) was added and the resulting mixture was stirred at 23° C. for 3.5 h. The mixture was concentrated. The residue was dissolved in MeOH and acidified to pH 2 by addition of TFA. The resulting solution was purified by reverse-phase HPLC (YMC Pack ODS-A column, gradient elution from 35 to 55% CH₃CN-water, both containing 0.1% TFA). The column eluant was lyophilized, yielding Compound 172 (36.6 mg) as a white powder. (TOF MS ES+) m/z 438 (M+H)⁺.

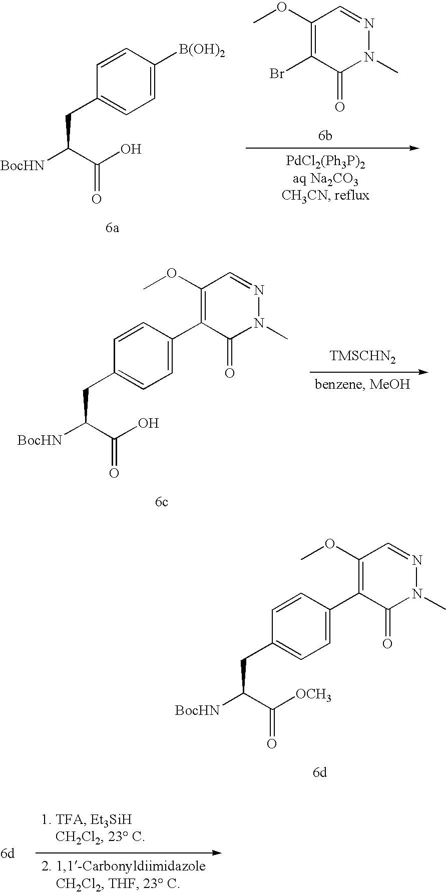

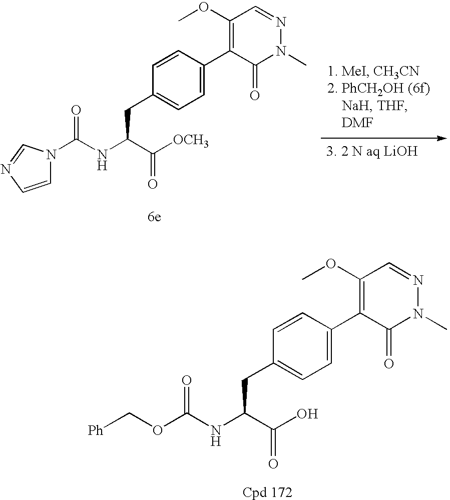

Other compounds of the present invention may be prepared by those skilled in the art by varying the starting materials, reagent(s) and conditions used. Using the procedure of Example 6, the following compounds were prepared without further purification:

| Cdp | MS (M + H⁺) |
|---|---|
| 111 | 446 |
| 171 | 418 |
| 175 | 404 |
| 184 | 390 |
| 201 | 444 |
| 203 | 480 |
| 204 | 506 |
| 208 | 430 |
| 209 | 452 |

### EXAMPLE 7

### (S)-2,6-Dichloro-N[2-[4-(5-methoxy-2-methyl-3-oxo-2,3-di hydro-pyridazin-4-yl)-phenyl]-1-(1H-tetrazol-5-yl)-ethyl]-benzamide, Cpd 106

To a solution of Compound 6d (2.07 g, 4.96 mmol) in a mixture of MeOH:tetrahydrofuran (1:1, 20 mL) was added 2 N aqueous LiOH (10 mL, 20 mmol). The resulting solution was stirred at 23° C. for 4 h. The mixture was partially concentrated to remove the organic solvents. The resulting solution was cooled to 0° C., then was acidified to pH 2 by addition of 2 N aqueous HCl. The acidified solution was extracted with DCM (4×20 mL). The combined organic extracts were dried (Na₂SO₄) and were concentrated. Di-tert-butyl dicarbonate (1.46 g, 6.67 mmol) and NH₄CO₃H (507 mg, 6.40 mmol) were added to the residue. The reaction vessel was flushed with N₂ prior to the sequential addition of acetonitrile (24 mL) and pyridine (249 μL, 3.08 mmol). The mixture was stirred at 23° C. for 19 h. The mixture was concentrated and the resulting white foam was purified by column chromatography (silica gel, gradient elution, 2-10% MeOH/DCM). Compound 7a was obtained as a white foam (1.76 g). (TOF MS ES+) m/z 403 (M+H)⁺.

Cyanuric chloride (Compound 7b, 518 mg, 2.81 mmol) was added to an ice-cold solution of Compound 7a (1.74 g, 4.32 mmol) in DMF. The solution was allowed to slowly warm to 23° C. and was stirred for 25 h. The mixture was partitioned between EtOAc (30 mL) and water (30 mL). The aqueous phase was extracted with EtOAc (3×30 mL). The combined organic extracts were dried (Na₂SO₄) and concentrated. The residue was purified by column chromatography (silica gel, gradient elution from 50-80% EtOAc/hexanes). Compound 7c was obtained as a white solid (1.38 g). (TOF MS ES+) m/z 385 (M+H)⁺.

Sodium azide (18.9 mg, 291 μmol), zinc bromide (164 mg, 728 μmol), iPrOH (0.33 mL), and water (0.33 mL) were added in sequence to Compound 7c (56.0 mg, 146 μmol). The resulting suspension was heated at reflux for 19 h, then stirred at 23° C. for 7 d. The mixture was concentrated and the residue purified by reverse-phase HPLC (YMC Pack ODS-A column, gradient elution from 5-25% acetonitrile-water, both containing 0.1% TFA). Compound 7d was obtained as a colorless oil (22.7 mg). (TOF MS ES+) m/z 328 (M+H)⁺.

Triethylamine (157 μL, 1.13 mmol) and Compound 1e (80.7 μL, 0.563 mmol) were added in sequence to a suspension of Compound 7d (219 mg, 0.512 mmol) in DCM (2.5 mL). The resulting suspension was stirred at 23° C. for 15 h. The mixture was concentrated and the resultant residue was suspended in MeOH and acidified with the addition of TFA. The resulting yellow solution was purified by reverse-phase HPLC (YMC Pack ODS-A column, gradient elution from 25-45% acetonitrile-water, both containing 0.1% TFA). Compound 106 was obtained as a colorless oil (52 mg). (TOF MS ES+) m/z 500 (M+H)⁺.

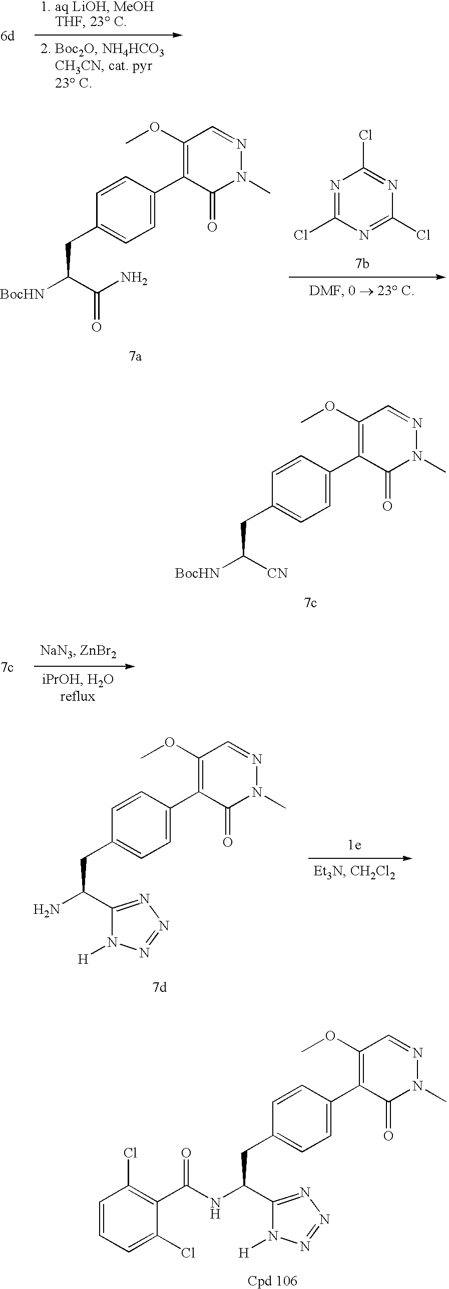

### EXAMPLE 8

### (S)-2,6-Dichloro-N{2-methanesulfonylamino-1-[4-(5-methoxy-2-methyl-3-oxo-2,3-dihydro-pyridazin-4-yl)-benzyl]-2-oxo-ethyl}-benzamide, Cpd 192

Dimethylaminopyridine (32.1 mg, 263 μmol), EDC (50.4 mg, 263 μmol, 1.25 equiv), and methanesulfonamide (25.0 mg, 263 μmol, 1.25 equiv) were added in sequence to a solution of Compound 17 (100 mg, 210 μmol) in DCM (1.0 mL). The resulting solution was stirred at 23° C. for 11 d. The resulting mixture was partitioned between DCM (5 mL) and 1 N HCl (aq) (5 mL). The organic phase was dried (Na₂SO₄), filtered, and concentrated. The residual white solid was purified initially by column chromatography (silica gel, gradient elution from 1 to 10% MeOH in DCM:HOAc, 99:1). A portion of the material obtained was further purified by preparative thin-layer chromatography (elution solvent: HOAc:MeOH:DCM, 1:10:89). Compound 192 was obtained as a colorless oil (10.8 mg). (MS ES+) m/z 553 (M+H)⁺.

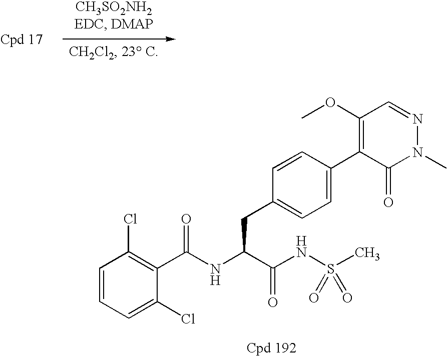

### EXAMPLE 9

### (S)-2,6-Dichloro-N-{1-hydroxycarbamoyl-2-methyl-3-oxo-2,3-dihydro-pyridazin-4-yl)-benzyl]-2-oxo-ethyl}-benzamide, Cpd 176

To a solution of Compound 17 (320.2 mg, 572 μmol) in DMF (3.0 mL) was added HOBt (118.1 mg, 874 μmol) followed by EDC hydrochloride (193.3 mg, 1.01 mmol). The resulting mixture was stirred at 23° C. for 5 min. Hydroxylamine hydrochloride (51.4 mg, 739 μmol) was then added followed by triethylamine (103.0 μL, 739 μmol). The mixture was stirred at 23° C. for 20 h. The mixture was partitioned between EtOAc (10 mL) and a saturated solution of NaHCO₃ (aq) (10 mL). The organic phase was dried (Na₂SO₄), filtered, and concentrated. The residual white solid was purified by reverse-phase HPLC (YMC Pack ODS-A column, gradient elution from 20 to 40% acetonitrile-water, both containing 0.1% TFA), yielding Compound 176 as a white powder (14.2 mg). (TOF MS ES+) m/z 491 (M+H)⁺.

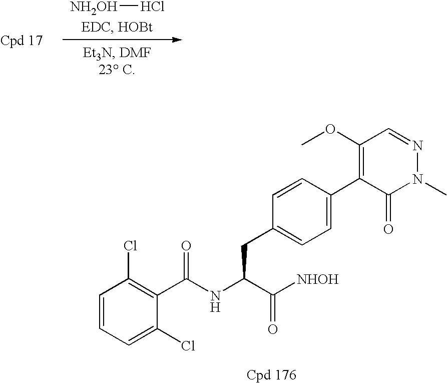

### EXAMPLE 10

### (S)-2,6-Dichloro-N{2-hydroxy-1-[4-(5-methoxy-2-methyl-3-oxo-2,3-dihydro-pyridazin-4-yl)-benzyl]-ethyl}-benzamide, Cpd 141

A solution of lithium borohydride in THF (2.0 M, 486 μL, 972 μmol) was added to a solution of Compound 1f (216.6 mg, 442 mmol) at 0° C. The resulting yellow solution was stirred at 0° C. for 30 min, then was allowed to warm to 23° C. and was stirred for an additional 3 h. Excess hydride was quenched by the addition of a saturated solution of NH₄Cl (aq). The resulting solution was concentrated and the residual white solid was partitioned between EtOAc (5 mL) and saturated NH₄Cl (aq) (5 mL). The aqueous phase was extracted with EtOAc (5 mL). The combined organic extracts were dried (Na₂SO₄), filtered, and concentrated. The residue was purified initially by column chromatography (silica gel, EtOAc), then by reverse-phase HPLC (YMC Pack ODS-A column, gradient elution from 20 to 40% acetonitrile-water, both containing 0.1% TFA). Compound 141 was obtained as a colorless oil (76.6 mg). (MS ES+) m/z 462 (M+H)⁺.

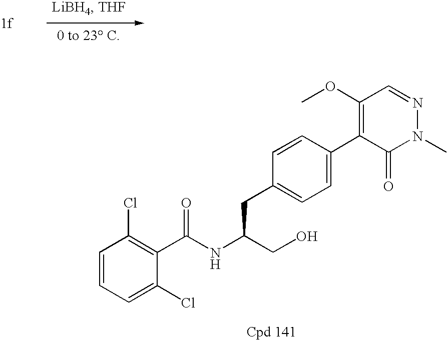

### EXAMPLE 11

### (R)-2-(2-tert-Butoxycarbonylamino-3-methyl-butyrylamino)-3-(S)-[4-(5-methoxy-2-methyl-3-oxo-2,3-dihydro-pyridazin-4-yl)-phenyl]-propionic acid, Cpd 81

A mixture of Compound 1d (200 mg, 0.63 mmol), Compound 11a (187 mg, 0.63 mmol), EDC (157 mg, 0.82 mmol), HOBt (153 mg, 1.13 mmol, and DIEA (219 μL, 1.26 mmol), in 20 mL of CH₂Cl₂ was allowed to stir at rt under an argon atmosphere for 30 h. The mixture was washed with 10% citric acid (aq) solution followed by saturated NaHCO₃ (aq) solution. The organic layer was dried (MgSO₄), filtered, and concentrated to yield Compound 11b (458 mg) as an amber oil.

To a solution of Compound 11b (20 mg, 0.03 mmol) 2 mL of 1:1 MeOH:H₂O was added LiOH—H₂O (8 mg, 0.18 mmol). After stirring for 23 h, the mixture was concentrated and purified on a preparative reverse phase HPLC (YMC Pack ODS-H80 column 100×20 mm, gradient elution from 2040% water-acetonitrile, both containing 0.1% TFA) to yield Compound 81 (7 mg) as a white powder. LC 100% @254 nm, 98% @214 nm; ¹H NMR (CD₃OD): δ 0.75 (d, 3H), 0.83 (d, 3H), 1.43 (s, 9H), 1.90 (m, 1H), 3.05 (m, 1H), 3.23 (m, 1H), 3.78 (s, 3H), 3.93 (s, 3H), 3.94 (m, 1H), 4.72 (m, 1H), 7.29 (d, 2H), 7.40 (d, 2H), 8.18(s, 1H).

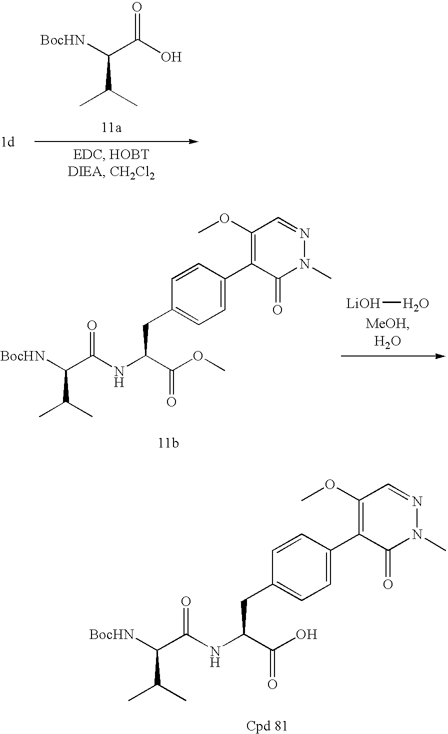

Other compounds of the present invention may be prepared by those skilled in the art by varying the starting materials, reagent(s) and conditions used. Using the procedure of Example 11, the following compounds were prepared without further purification:

| Cpd | MS (M + H⁺) |
|---|---|
| 216 | 389 |
| 217 | 388 |
| 222 | 529 |
| 223 | 515 |
| 229 | 432 |
| 230 | 478 |

### EXAMPLE 12

### 2-(2-Amino-3-methyl-butyrylamino)-3-[4-(5-methoxy-2-methyl-3-oxo-2,3-dihydro-pyridazin-4-yl)-phenyl]-propionic acid, Cpd 181

To a solution of Compound 11b (458 mg, 0.89 mmol) in 5 mL of DCM was added 2 mL of TFA. The resulting mixture was allowed to stir at rt for 1.5 h. The mixture was concentrated, the residue was dissolved in MeOH, and the mixture was concentrated again. The residue was purified on a preparative reverse phase HPLC (YMC Pack ODS-H80 column 100×20 mm, gradient elution from 5-25% acetonitrile-water, both containing 0.1% TFA) to obtain Compound 12a (192 mg, 0.36) as a white foam.

To a solution of 20 mg (0.03 mmol) of 12a in 4 mL of 1:1 MeOH: H₂O was added 5 mg (0.12 mmol) of LiOH—H₂O. The resulting mixture was allowed to stir at rt overnight. The mixture was acidified by the addition of several drops of TFA and concentrated to 1 mL. The product was purified by preparative reverse phase HPLC (YMC Pack ODS-H80 column 100×20 mm, gradient elution from 5-25% water-acetonitrile, both containing 0.1% TFA) to obtain Compound 181 (8.8 mg) as a white powder. LC 96% desired R valine isomer, 4% S valine isomer; ¹H NMR (CD₃OD): δ 0.70 (d, 3H), 0.82 (d, 3H), 1.97 (m, 1H), 2.99 (m, 1H), 3.33 (m, 1H), 3.62 (d, 1H), 3.78 (s, 3H), 3.92 (s, 3H), 4.87 (m, 1H), 7.31 (d, 2H), 7.38 (d, 2H), 8.17 (s, 1H).

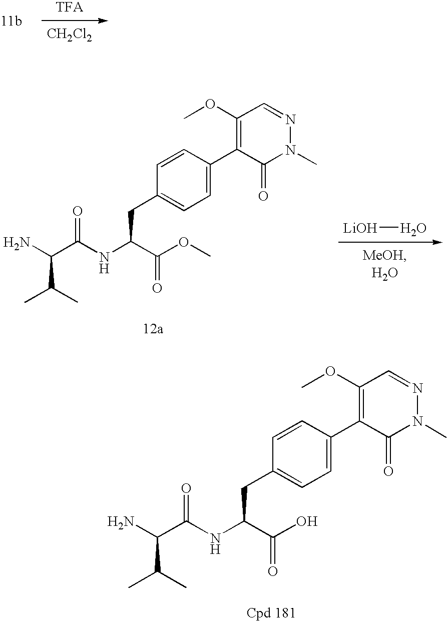

Other compounds of the present invention may be prepared by those skilled in the art by varying the starting materials, reagent(s) and conditions used. Using the procedure of Example 12, the following compounds were prepared:

| Cpd | MS (M + H⁺) |
|---|---|
| 123 | 417 |
| 128 | 608 |
| 159 | 403 |
| 166 | 401 |
| 181 | 403 |
| 210 | 502 |
| 221 | 403 |

### EXAMPLE 13

### 3-[4-(5-Methoxy-2-methyl-3-oxo-2,3-dihydro-pyridazin-4-yl)-phenyl]-2-[3-methyl-2-(2-thiophen-3-yl-acetylamino)-butyrylamino]-propionic acid, Cpd 107

A solution of Compound 12a (17 mg, 0.027 mmol), Compound 13a (4 mg, 0.03 mmol), EDC (8 mg, 0.04 mmol), HOBt (7 mg, 0.054 mmol), and DIEA (16 μL, 0.09 mmol) in 5 mL of CH₂Cl₂ was allowed to stir at rt overnight. The mixture was washed with 10% citric acid (aq) followed by saturated NaHCO₃ (aq) solution. The organic layer was dried (MgSO₄), filtered, and concentrated to yield Compound 13b (12 mg).

To a solution of Compound 13b (12 mg, 0.022 mmol) in 3 mL of 2:1 MeOH: H₂O was added LiOH—H₂O (3 mg, 0.06 mmol). After stirring for 1.5 h, the mixture was acidified with several drops of TFA and purified by preparative reverse phase HPLC (YMC Pack ODS-H80 column 100×20 mm, gradient elution from 2040% water-acetonitrile, both containing 0.1% TFA) to yield Compound 107 (2.5 mg) as a white powder.

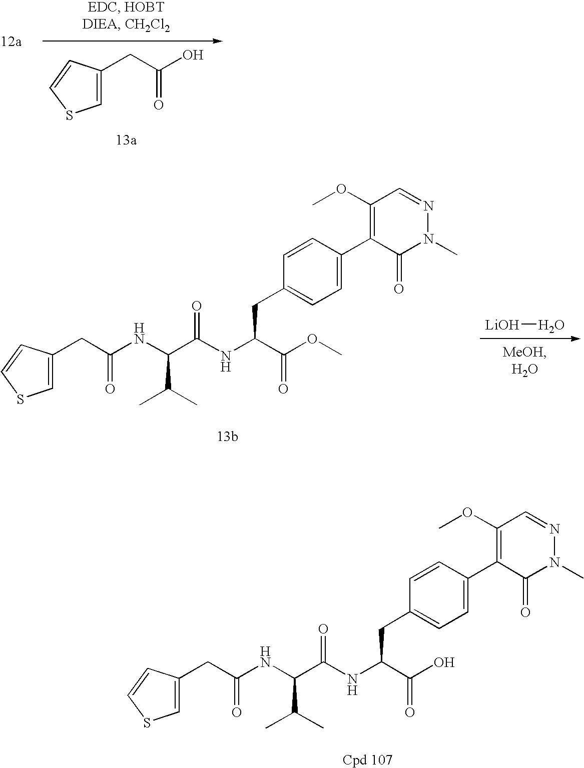

Other compounds of the present invention may be prepared by those skilled in the art by varying the starting materials, reagent(s) and conditions used. Using the procedure of Example 13, the following compounds were prepared without further purification:

| Cpd | MS (M + H⁺) |
|---|---|
| 227 | 429 |

### EXAMPLE 14

### 2-(2-Isopropylamino-3-methyl-butyrylamino)-3-[4-(5-methoxy-2-methyl-3-oxo-2,3-dihydro-pyridazin-4-yl)-phenyl]-propionic acid, Cpd 153

To a solution of Compound 12a (55 mg, 0.08 mmol) in 5 mL of THF was added acetone (6.3 μL, 0.08 mmol) and Na(OAc)₃BH (25 mg, 0.12 mmol). The resulting mixture was allowed to stir under an argon atmosphere for 4 h. The mixture was concentrated, and the residue was taken up in CH₂Cl₂ and washed with Na₂CO₃ (aq) solution. The aqueous layer was separated and washed (2×) with CH₂Cl₂. The combined organics were dried (MgSO₄), filtered, and concentrated to Compound 14a (40 mg, 0.08 mmol) as a clear oil.

To a solution of Compound 14a (40 mg, 0.08 mmol) in 4 mL of 1:1 MeOH: H₂O was added LiOH—H₂O (7 mg, 0.16 mmol). The resulting solution was allowed to stir at rt for 2.5 h. The mixture was concentrated and purified by preparative reverse phase HPLC (YMC Pack ODS-H80 column 100×20 mm, gradient elution from 5-25% water-acetonitrile, both containing 0.1% TFA) to give Compound 153 (6 mg, 0.01 mmol) as a white powder. ¹H NMR (CD₃OD): δ 0.65 (d, 3H), 0.70 (d, 3H), 1.22 (m, 6H), 1.86 (m, 1H), 2.88 (m, 1H), 3.28 (m, 1H), 3.56 (d, 1H), 3.68 (s, 3H), 3.83 (s, 3H), 4.70 (m, 1H), 7.21 (d, 2H), 7.30 (d, 2H), 8.08 (s, 1H).

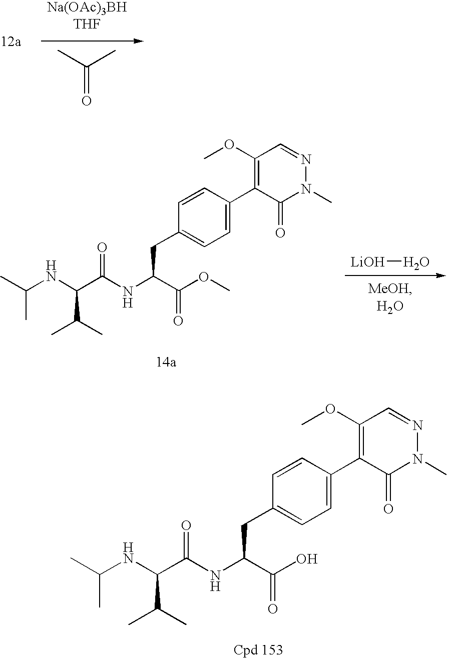

Other compounds of the present invention may be prepared by those skilled in the art by varying the starting materials, reagent(s) and conditions used. Using the procedure of Example 14, the following compounds were prepared:

| Cpd | MS (M + H⁺) |
|---|---|
| 146 | 429 |
| 179 | 544 |
| 189 | 485 |
| 218 | 431 |
| 220 | 529 |
| 224 | 527 |
| 225 | 455 |
| 226 | 457 |

### EXAMPLE 15

### 3-[4-(5-Methoxy-2-methyl-3-oxo-2,3-dihydro-pyridazin-4-yl)-phenyl]-2-(3-methyl-2-pyrrol-1-yl-butyrylamino)-propionic acid, Cpd 87

A solution of Compound 15a (14 μL, 0.11 mmol) in 0.5 mL of 0.1 M HCl was heated to 100° C. for 40 min. The mixture was cooled to rt. A solution of Compound 12a (55 mg, 0.01 mmol) in 5 mL of CH₂Cl₂ was added and the resulting mixture was allowed to stir at rt for 2 h. The mixture was washed with saturated NaHCO₃ (aq) solution and separated. The aqueous layer was washed with additional CH₂Cl₂. The combined organic extracts were dried (MgSO₄), filtered and concentrated to Compound 15b (43 mg) as a clear oil.

To a solution of Compound 15b (43 mg, 0.092 mmol) in 3 mL of 1:1 MeOH:H₂O was added LiOH—H₂O (12 mg, 0.3 mmol). The solution was allowed to stir at rt overnight. The solution was concentrated and purified on a preparative reverse phase HPLC system (YMC Pack ODS-H80 column 100×20 mm, gradient elution from 20-40% water-acetonitrile, both containing 0.1% TFA) to yield Compound 87 (19 mg) as a white powder. LC 100%; ¹H NMR (CD₃OD): δ 0.53 (d, 3H), 0.66 (d, 3H), 2.20 (m, 1H), 2.92 (m, 1H), 3.14 (dd, 1H), 3.21 (m, 1H), 3.69 (s, 3H), 3.86 (s, 3H), 3.93 (d, 1H), 4.52 (m, 1H), 5.92 (s, 1H), 6.65 (s, 2H), 7.08 (d, 2H), 7.22 (d, 2H), 8.08 (s, 1H), 8.39 (m, 1H).

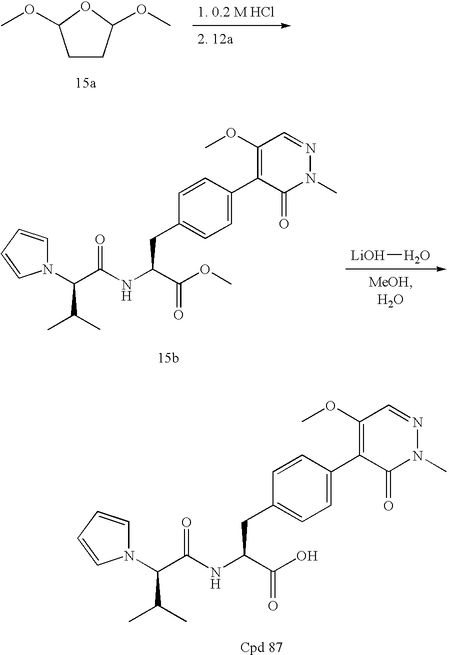

### EXAMPLE 16

### 2-[2(2,5-Dimethyl-pyrrol-1-yl)-3-methyl-butyrylamino]-3-[4-(5-methoxy-2-methyl-3-oxo-2,3-dihydro-pyridazin-4-yl)-phenyl]-propionic acid, Cpd 33

The (R,S)-2-(2-amino-3-methyl-butyrylamino)-3-[4-(5-methoxy-2-methyl-3-oxo-2,3-dihydro-pyridazin-4-yl0-phenyl]-propionic acid, methyl ester, di-TFA salt, prepared according to Example 12 (644 mg, 1 mmol) was suspended in 50 mL of toluene with acetonylacetone (230 mg). The reaction was equipped with a Dean-Stark trap, then heated to reflux under an argon atmosphere for 2 h. The mixture was cooled to rt and the volatile solvent was evaporated. The residue was subjected to column chromatography (silica gel, 0-10% MeOH in CHCl₃) to provide Compound 16a (278 mg). ¹H NMR (CD₃OD): δ 0.56 (m, 3H), 1.00-1.13 (dd, 3H), 2.06 (s, 3H), 2.17 (s, 3H), 2.56 (m, 1H), 3.00 (m, 1H), 3.76 (s, 3H), 3.94 (s, 3H), 7.03 (m, 1H), 7.18 (m, 1H), 7.32 (m, 2H), 8.20 (s, 1H).

Compound 16a was hydrolyzed to Compound 33 by the method described in Example 15. Compound 33 was isolated by HPLC (YMC Pack ODS-H80 column 100×20 mm, gradient elution from 30-50% water-acetonitrile, both containing 0.1% TFA). MS 481 (M+H).

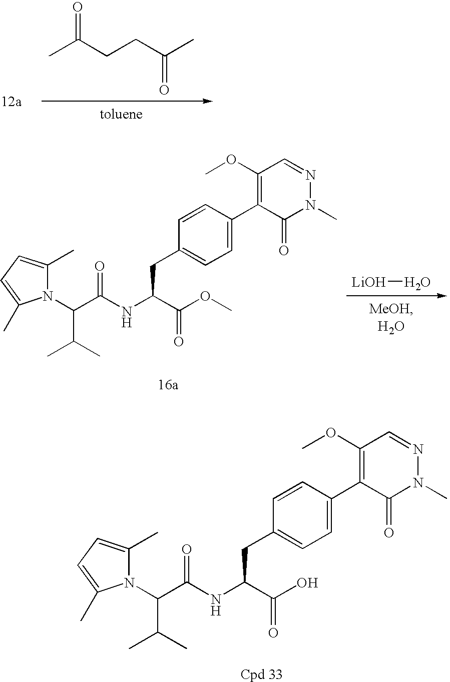

Other compounds of the present invention may be prepared by those skilled in the art by varying the starting materials, reagent(s) and conditions used. Using the procedure of Example 16, the following compounds were prepared:

| Cpd | MS (M + H⁺) |
|---|---|
| 213 | 481 |
| 214 | 481 |
| 215 | 481 |

### EXAMPLE 17

### (S)-2-(2,6-Dichloro-benzoylamino)-3-[4-(5-difluoromethoxy-2-methyl-3-oxo-2,3-dihydro-pyridazin-4-yl)-phenyl]-propionic acid, Cpd 112

Compound 1e (1.47 mL, 10.2 mmol) was added to a mixture of Compound 1a (4-borono-L-phenylalanine) (2.04 g, 9.76 mmol) and Na₂CO₃ (2.07 g, 19.5 mmol) in acetonitrile:water (1:1, 40 mL) at 50° C. The resulting mixture was stirred at 50° C. for 1 h, then was cooled to 0° C. and was acidified to pH 2 by addition of concentrated HCl (aq). The suspension was stirred at 0° C. for 30 min and the precipitated solid was collected by vacuum filtration and was washed with water. The white solid was dried in a vacuum oven at 50° C., affording Compound 17a (2.65 g). ¹H NMR (CD₃OD) δ 7.55 (d, 2H, J=7.6 Hz), 7.28-7.40 (m, 5H), 4.95 (dd, 1H, J=9.3, 4.7 Hz), 3.30 (dd, 1H, J=13.9, 5.3 Hz), 3.03 (dd, 1H, J=14.1, 9.4 Hz).

A pressure tube was charged sequentially with Compound 17b (Cho, S.-D.; Choi, W.-Y.; Yoon, Y.-J. J. Heterocycl. Chem. 1996, 33, 1579-1582) (1.29 g, 6.28 mmol), chlorodifluoroacetic acid sodium salt (1.15 g, 7.54 mmol), and NaOH (314 mg, 7.85 mmol). The vessel was purged with nitrogen, and DMF (3.0 mL) was added. The mixture was heated to 130° C. for 1 h, then was allowed to cool to 23° C. The mixture was diluted with EtOAc (50 mL) and the resulting solution was washed with a saturated solution of NaCl (aq) (2×50 mL). The organic phase was dried (Na₂SO₄), filtered, and concentrated, to yield a tan solid which was purified by column chromatography (silica gel, gradient elution from 50 to 70% EtOAc-hexanes). Compound 17c was obtained as an off-white solid (1.09 g). (MS ES+) m/z 255 (M+H)⁺.

To a mixture of Compound 17a (370 mg, 0.968 mmol, 1 equiv), Compound 17c (0.218 g, 1.06 mmol, 1.1 equiv) and transdichloro(bistriphenylphosphine)palladium (II) (33.9 mg, 0.0484 mmol, 0.05 equiv) were added in sequence an aqueous solution of sodium carbonate (2 M, 2 mL, 4 mmol, 4 equiv) and acetonitrile (2 mL). The resulting suspension was heated at reflux under a nitrogen atmosphere for 1 h, then was allowed to cool to 23° C. The mixture was partially concentrated, to remove organic solvent. The resulting mixture was diluted with half-saturated aqueous sodium bicarbonate (20 mL) and was washed with ether (20 mL). The aqueous extract was cooled to 0° C. and was acidified to pH 2 by addition of 1 N aqueous hydrochloric acid. The precipitated white solid was collected by vacuum filtration. The crude product was purified by reverse-phase HPLC (YMC Pack ODS-A column, gradient elution from 35 to 55% acetonitrile-water, both containing 0.1% TFA) affording Compound 112 (305.0 mg). (MS ES+) m/z 512 (M+H)⁺.

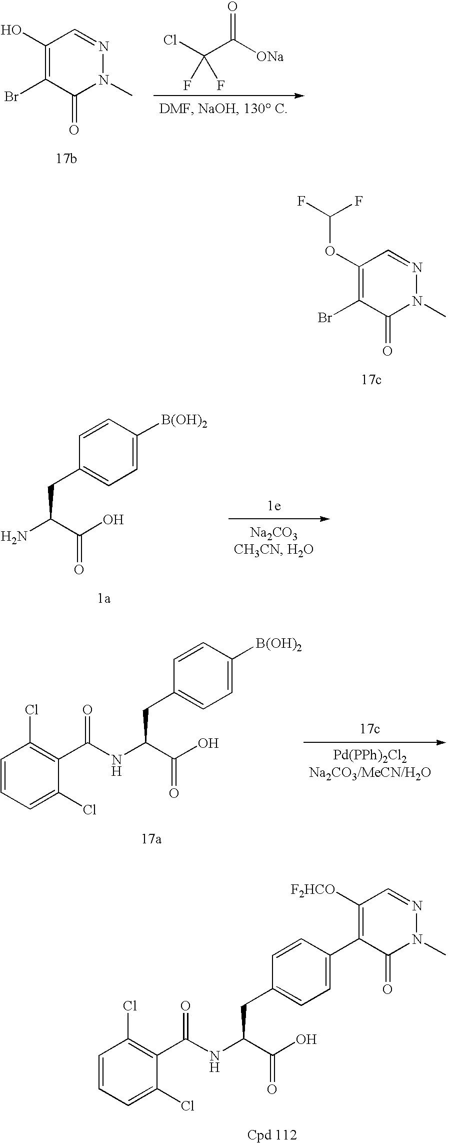

Other compounds of the present invention may be prepared by those skilled in the art by varying the starting materials, reagent(s) and conditions used. Using the procedure of Example 17, the following compounds were prepared without further purification:

| Cpd | MS (M + H⁺) | Cpd | MS (M + H⁺) |
|---|---|---|---|
| 120 | 434 | 190 | 389 |

### EXAMPLE 18

### (S)-2-(2,6-Dichloro-benzoylamino)-3-[4-(2-methyl-3-oxo-5-phenyl-2,3-dihydro-pyridazin-4-yl)-phenyl]-propionic acid, Cpd 63

Phenylboronic acid (Compound 18a, 61 mg, 0.50 mmol) was dissolved in 1 M Na₂CO₃ (1 mL) and then mixed with Compound 18b (180 mg, 1.0 mmol) in DMF (1 mL). Pd(PEt₃)₂Cl₂ (10 mg, 0.024 mmol) was added and the resulting slurry was stirred at rt for 5 h. The crude mixture was concentrated to dryness, treated with water (2 mL), and extracted with DCM (3×2 mL). The DCM extract was concentrated to residue and purified by reverse phase HPLC (0.1% TFA H₂O/MeCN, 20-40% gradient). Compound 18c was obtained as a white solid (85 mg). mp 131-133° C.; ¹H NMR (CDCl₃, 300 MHz) δ 7.76 (s, 1H), 7.50 (s, 5H), 3.89 (s, 3H); MS m/z: 221 (M+H⁺).

Compound 17a and 18c were coupled to yield Compound 63 by the method described in Example 1 for the palladium-catalyzed coupling of Compounds 1a and 1b.

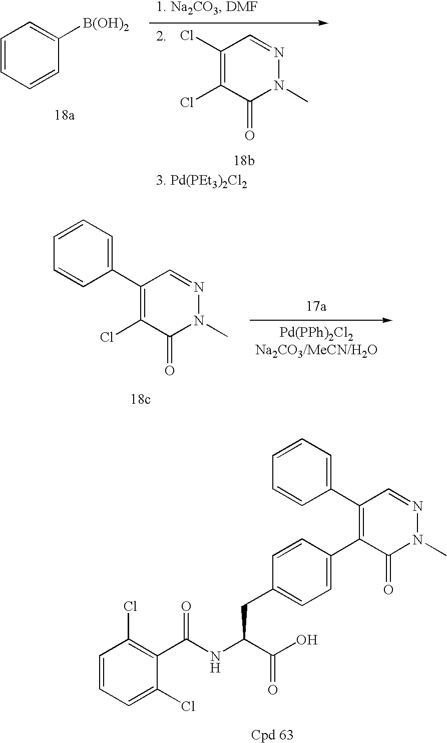

Other compounds of the present invention may be prepared by those skilled in the art by varying the starting materials, reagent(s) and conditions used. Using the procedure of Example 18, the following compounds were prepared without further purification:

| Cpd | MS (M + H⁺) | Cpd | MS (M + H⁺) |
|---|---|---|---|
| 16 | 565 | 47 | 552 |
| 59 | 540 | 105 | 528 |
| 14 | 600 | 67 | 547 |
| 71 | 606 | 64 | 590 |
| 27 | 566 | 65 | 640 |

### EXAMPLE 18-1

### 2-(2,6-Dichloro-benzoylamino)-3-[4-(5-ethyl-2-methyl-3-oxo-2,3-dihydro-pyridazin-4-yl)-phenyl]-propionic acid, Cpd 101

Compound 18b (0.45 g, 2.5 mmol) was added to Et₂Zn in hexanes (1.0 M, 9 mL) at 0° C. The resulting mixture was heated to 60° C. in an oil bath for 4 h, before being quenched with water and treating with CH₂Cl₂. The insoluble materials were filtered off, and the CH₂Cl₂ filtrate was concentrated and purified by HPLC to give Compound 18-1a as a clear liquid (47 mg). MS m/z: M+1=173.

Using the procedure described in Example 1 for converting Compound 1a to Compound 1c and substituting 18-1 a for 1 b, and Compound 17a for Compound 1a, Compound 18b was converted to Compound 101. MS m/z: M+1=474.

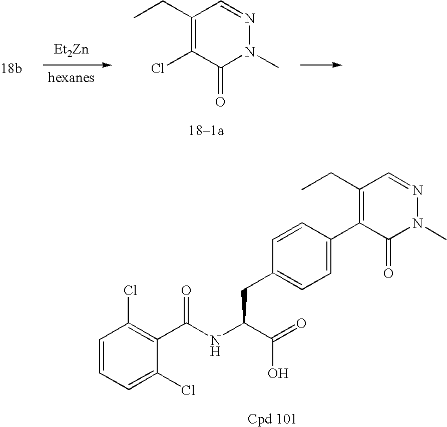

Other compounds of the present invention may be prepared by those skilled in the art by varying the starting materials, reagent(s) and conditions used. Using the procedure of Example 18-1, the following compounds were prepared without further purification:

| Cpd | MS (M + H⁺) | Cpd | MS (M + H⁺) |
|---|---|---|---|
| 4 | 490 | 5 | 519 |
| 10 | 575 | 11 | 520 |
| 20 | 531 | 36 | 543 |
| 37 | 544 | 39 | 544 |
| 49 | 480 | 51 | 515 |
| 56 | 538 | 78 | 534 |
| 85 | 518 | 92 | 585 |
| 117 | 552 | 197 | 523 |
| 165 | 544 | 228 | 518 |

### EXAMPLE 19

### (S)-2-(2,6-Dichloro-benzoylamino)-3-{4-[2-(2-hydroxy-ethyl)-3-oxo-5-(5-thiophen-2-yl-pyrazol-1-yl)-2,3-dihydro-pyridazin-4-yl]-phenyl}-propionic acid, Cpd 15

2-Hydroxyethylhydrazine (13.2 mL, 195.0 mmol) was added to a solution of mucobromic acid (Compound 19a, 38.68 g, 150.0 mmol) in EtOH (128 mL) at 5° C. The internal temperature rose to 10° C. during the addition. The mixture was stirred at 0° C. for 1 h, then was allowed to warm to 23° C., before further heating to reflux for 2 h. The mixture was allowed to cool to 23° C. and was concentrated. A portion of the resulting black oil was purified by column chromatography (silica gel, gradient elution from 50 to 75% EtOAc-hexanes). Compound 19b was obtained as a tan solid (22.93 g). ¹H NMR (CDCl₃) δ 7.85 (s, 1H), 4.38 (t, 2H, J=5.1 Hz), 4.04 (t, 2H, J=5.1 Hz), 2.41 (br s, 1H).

A solution of NaOMe in MeOH (30 wt. %, 4.85 mL, 25.8 mmol) was added to an ice-cold solution of Compound 19b (7.00 g, 23.5 mmol) in MeOH (40 mL). The resulting mixture was allowed to slowly warm to 23° C. and was stirred for 21 h. The mixture was concentrated and the residual off-white solid was partitioned between CH₂Cl₂ (100 mL) and a saturated solution of NaCl (aq) (100 mL). A white solid precipitated from the resulting mixture. The solid was collected by vacuum filtration, affording Compound 19c as a white powder (4.73 g). (MS ES+) m/z 249 (M+H)⁺.

To a mixture of Compound 19c (1.88 g, 4.91 mmol), Compound 17a (1.35 g, 5.40 mmol) and trans-dichloro(bistriphenylphosphine)palladium (II) (172 mg, 0.246 mmol) were added in sequence an aqueous solution of Na₂CO₃ (2 M, 10 mL, 20 mmol) and CH₃CN (10 mL). The resulting suspension was heated at reflux under a nitrogen atmosphere for 1 h, then was allowed to cool to 23° C. The mixture was partially concentrated to remove organic solvent. The resulting mixture was diluted with half-saturated aqueous NaHCO₃ (50 mL) and was washed with Et₂O (50 mL). The aqueous extract was cooled to 0° C. and was acidified to pH 2 by addition of 1 N aqueous HCl. The precipitated white solid was collected by vacuum filtration, affording Compound 15 (2.11 g). (MS ES+) m/z 506 (M+H)⁺.

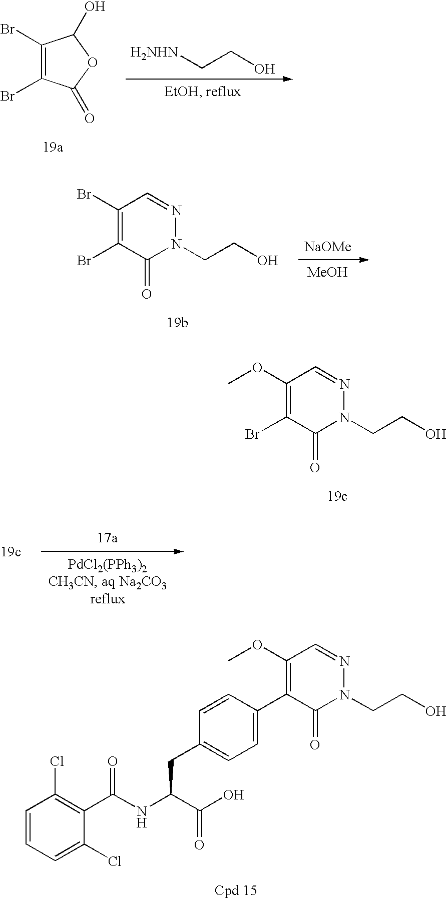

### EXAMPLE 19-1

### (S)-2-(2,6-Dichloro-benzoylamino)-3{4-[2-(2-hydroxy-ethyl)-5-methoxy-3-oxo-2,3-dihydro-pyridazin-4-yl]-phenyl}-propionic acid, Cpd 61

A mixture of 19b (0.30 g, 1.0 mmol) and morpholine (0.33 mL, 2.5 mmol) in water (1.2 mL) was heated to 120° C. in an oil bath for 4 h, and then concentrated to a residue. The residue was extracted with MeCN, and the insoluble material was removed by filtration. The filtrate was concentrated to a residue and treated with water (0.5 mL). The precipitate was collected by filtration, and washed with water to give Compound 19-1a as a white solid (0.06 g). ¹H NMR (CDCl₃, 300 MHz) δ 7.57 (s, 1H), 4.39 (t, 2H), 4.00 (t, 2H), 3.88 (t, 4H), 3.41 (t, 4H); MS m/z: 304 (M⁺).

Using the method of Example 19 for the conversion of 19c to Compound 15, Compound 19-1a was converted to the title Compound 61. MS m/z 561 (M+H)⁺.

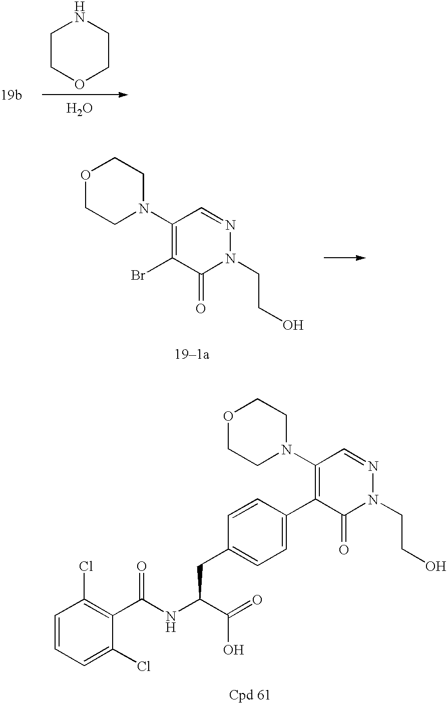

### EXAMPLE 20

### (S)-2-(2,6-Dichloro-benzoylamino)-3-{4-[2-(2-hydroxy-ethyl)-3-oxo-5-(5-thiophen-2-yl-pyrazol-1-yl)-2,3-di hydro-pyridazin-4-yl]-phenyl}-propionic acid, Cpd 131

Sodium hydride (60% dispersion in mineral oil, 42 mg, 1.05 mmol) was added to a solution of Compound 20a (157.7 mg, 1.05 mmol) in THF (1 mL). To the resulting suspension was added Compound 15 (106 mg, 210 μmol). The mixture was heated by microwave irradiation (CEM Explorer, 100° C., 10 min). To the mixture was added aqueous HCl solution (1.0 N, 1.5 mL). The resulting mixture was filtered through a plug of Celite (Varian Chem Elut), which was washed with 1% AcOHY CH₂Cl₂ (10 mL). The filtrate was concentrated and the residue was purified by reverse-phase HPLC, yielding Compound 131 (13.2 mg). (MS ES+) m/z 624 (M+H)⁺.

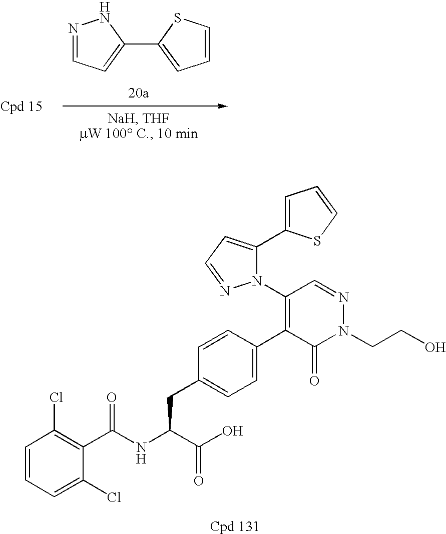

Other compounds of the present invention may be prepared by those skilled in the art by varying the starting materials, reagent(s) and conditions used. Using the procedure of Example 20, the following compounds were prepared without further purification:

| Cpd | MS (M + H⁺) | Cpd | MS (M + H⁺) |
|---|---|---|---|
| 32 | 526 | 88 | 292 |
| 69 | 540 | 94 | 620 |
| 79 | 562 | 148 | 594 |
| 82 | 590 | | |

### EXAMPLE 21

### (S)-3-[4-(2-tert-Butyl-3-oxo-5-p-tolyloxy-2,3-dihydro-pyridazin-4-yl)-phenyl]-2-(2,6-dichloro-benzoylamino)-propionic acid, Cpd 196

To a suspension of Compound 1a (13.6 mg, 65.1 μmol) in a mixture of aqueous sodium carbonate (2M, 0.25 mL) and acetonitrile (0.25 mL) was added Compound 1e (10.3 μL, 71.9 μmol). The mixture was stirred at 50° C. for 30 min prior to the addition of trans-dichloro(bistriphenylphosphine) palladium (II) (2.3 mg, 3.3 μmol) and Compound 21a (21.0 mg, 71.7 μmol). The resulting suspension was heated by microwave irradiation (CEM Explorer, 150° C., 6 min). The resulting mixture was acidified to pH 2 by addition of TFA and was concentrated and resuspended in a mixture of 1% HOAc-CH₂Cl₂ (500 μL) and water (100 μL). The resulting mixture was filtered through a plug of Celite (Varian Chem Elut), which was washed with 1% HOAc-CH₂Cl₂ (4×1.2 mL). The filtrate was concentrated and the residue was purified by reverse-phase HPLC, affording Compound 196 as a colorless oil (9.3 mg). (MS ES+) m/z 594.6 (M+H)⁺.

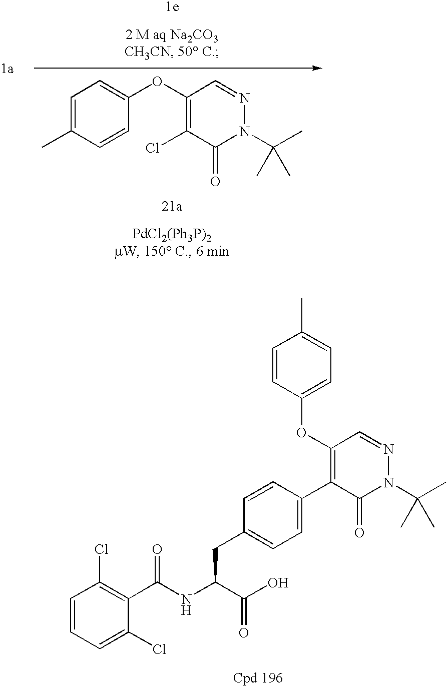

Other compounds of the present invention may be prepared by those skilled in the art by varying the starting materials, reagent(s) and conditions used. Using the procedure of Example 21, the following compounds were prepared without further purification:

| Cpd | MS (M + H⁺) | Cpd | MS (M + H⁺) |
|---|---|---|---|
| 18 | 518 | 142 | 533 |
| 29 | 546 | 155 | 532 |
| 46 | 462 | 160 | 556 |

### EXAMPLE 22

Using the method described in Example 5, the following compounds were prepared from Compound 15:

| Cpd | MS (M + H⁺) | Cpd | MS (M + H⁺) |
|---|---|---|---|
| 22 | 605 | 35 | 546 |
| 23 | 590 | 43 | 561 |
| 24 | 535 | 53 | 547 |
| 26 | 536 | 72 | 575 |
| 30 | 583 | | |

### EXAMPLE 23

### 2-[2-(3-Benzoyl-2,5-dimethyl-pyrrol-1-yl)-3-methyl-butyrylamino]-[4-(5-methoxy-2-methyl-3-oxo-2,3-dihydro-pyridazin-4-yl)-phenyl]-propionic acid, Cpd 211

Compound 23b was prepared using the methodology of U.S. Pat. No. 3,998,844.

Compound 23a was prepared using the procedure described in Example 16, using racemic materials. Compound 23a (1 g, 0.002 mol) was heated to reflux with 2 equivalents of benzoyl chloride (560 mg) in 5 mL of xylene for 36 h. The reaction mixture was then cooled, the solvent was removed in vacuo, and the residue was purified by column chromatography (silica, heptane-EtOAc, 50 to 100%) to yield Compound 23b (418 mg).

Compound 23b was hydrolyzed by the method described in Example 15. The residue was purified by reverse phase HPLC to yield Compound 211 as white powder.

HPLC analysis indicated a 1:1 mixture of diastereomers; MS 555 (M−H); 557 (M+H).

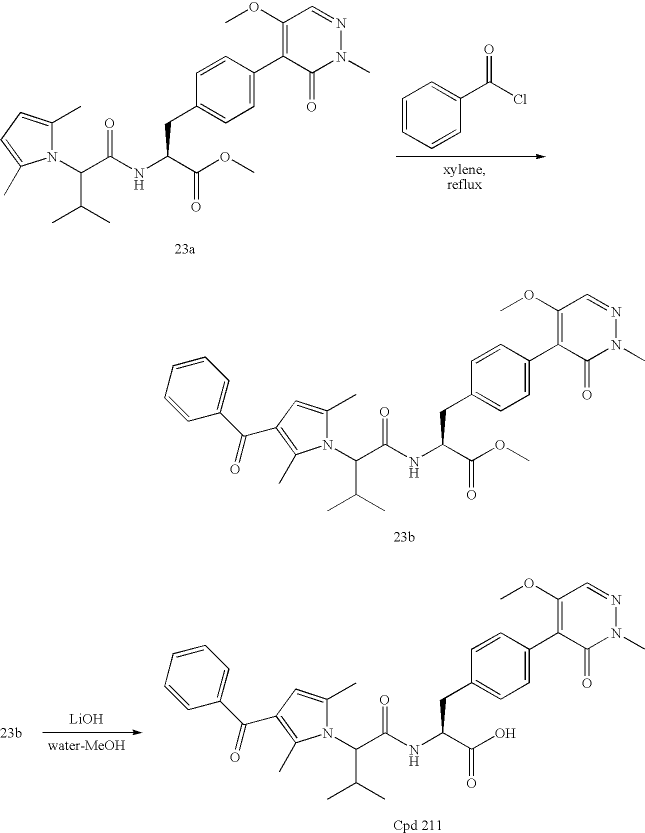

### EXAMPLE 24

### (S)-2-(2,6-Dichloro-benzoylamino)-3-[4-(2-methyl-3-oxo-2,3,6,7-tetrahydro-[1,4]dioxino[2,3-c]pyridazin-4-yl)-phenyl]-propionic acid, Cpd 6

A mixture of Compound 24a (0.45 g, 2.0 mmol), ethylene glycol (0.12 mL, 2.2 mmol), and K₂CO₃ (0.61 g, 4.4 mmol) in MeCN (20 mL) was heated at 100° C. in an oil bath for 3 h. The insoluble materials were removed by filtration. The filtrate was concentrated and treated with H₂O and CH₂Cl₂. The CH₂Cl₂ extract was concentrated and purified by HPLC to give Compound 24b as an off-white solid (18 mg). ¹H NMR (CDCl₃, 300 MHz) δ 4.45 (s, 4H), 3.71 (s, 3H); MS m/z: M+1=203.

Using the procedure described in Example 1 for converting Compound 1a to Compound 1c and substituting 24b for 1b, and Compound 17a for Compound 1a, Compound 24b was converted to Compound 6. MS m/z: M+1=504.

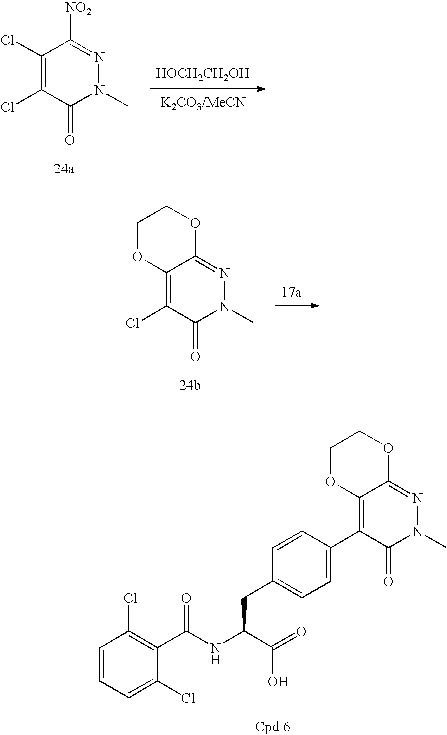

### EXAMPLE 25

### (S)-2-(2,6-Dichloro-benzoylamino)-3-[4-(2-methyl-3-oxo-3,5,6,7-tetrahydro-2H-pyridazino[3,4-b][1,4]oxazin-4-yl)-phenyl]-propionic acid, Cpd 13

A mixture of Compound 24a (0.23 g, 1.0 mmol) and ethanolamine (0.15 mL, 2.5 mmol) in EtOH (3 mL) was heated under microwave at 150° C. for 10 min. A solid formed upon cooling and was collected by filtration to provide Compound 25b (0.13 g).

Using the procedure described in Example 1 for converting Compound 1a to Compound 1c and substituting 25b for 1b, and Compound 17a for Compound 1a, Compound 25b was converted to Compound 13 as its TFA salt (18 mg). ¹H NMR (CD₃OD) δ: 7.45-7.26 (m, 7H), 4.94 (dd, 1H), 4.32 (t, 2H), 3.57, 3.35 (t, 2H), (s, 3H), 3.28 (dd, 1H), 3.16 (dd, 1H); MS m/z: M+1=503.

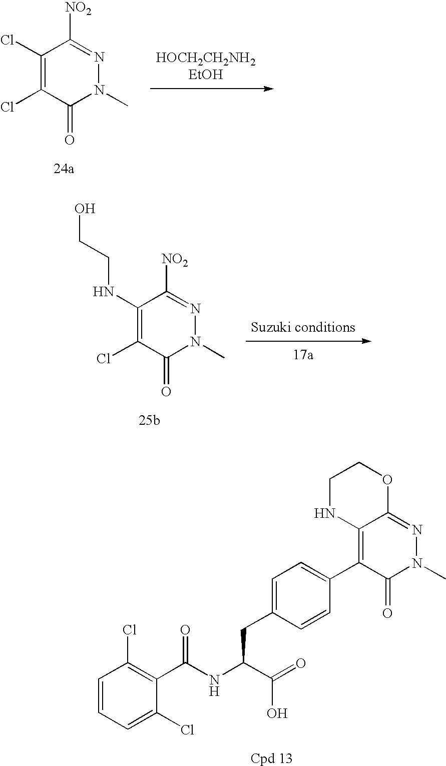

### EXAMPLE 26

### (S)-3-[4-(5-Chloro-2-cyclopropylmethyl-3-oxo-2,3-dihydro-pyridazin-4-yl)-phenyl]-2-(2,6-dichloro-benzoylamino)-propionic acid, Cpd 93

A mixture of Compound 26a (1.65 g, 10 mmol), (bromomethyl)cyclopropane (2.0 mL, 20 mmol), and K₂CO₃ (2.76 g, 20 mmol) in DMF (40 mL) was stirred at rt for 2 h. The mixture was concentrated and treated with H₂O and CH₂Cl₂. The CH₂Cl₂ extract was washed with water and concentrated to a yellow solid, Compound 26b (1.4 g). ¹H NMR (CDCl₃, 300 MHz) δ 7.78 (s, 1H), 4.04 (d, 2H), 1.35 (m, 1H), 0.56 (m, 2H), 0.43 (m, 2H); MS m/z: M+1=219.

Using the procedure described in Example 1 for converting Compound 1a to Compound 1c and substituting 26b for 1b, and Compound 17a for Compound 1a, Compound 26b was converted to Compound 93. MS m/z: M+1=520.

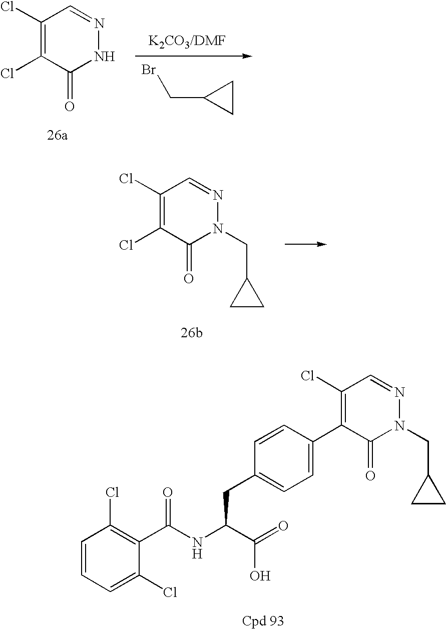

### EXAMPLE 27

### (S)-2-(2,6-Dichloro-benzoylamino)-3-[4-(2,5-dimethyl-3-oxo-2,3-dihydro-pyridazin-4-yl)-phenyl]-propionic acid, Cpd 109 and (S)-2-(2,6-dichloro-benzoylamino)-3-[4-(5ethoxycarbonylmethyl-2-methyl-3-oxo-2,3-dihydro-pyridazin-4-yl)-phenyl]-propionic acid, Cpd 121

Diethyl malonate (Compound 27a) (0.46 mL, 3.0 mmol) in THF (10 mL) was treated with 60% NaH (0.14 g, 3.5 mmol) at rt for 20 min before addition of Compound 18b (0.35 g, 2.0 mmol) in THF (10 mL). The mixture was stirred overnight and then acidified with TFA. The mixture was concentrated and purified by HPLC to give Compound 27b as a clear oil (0.23 g). ¹H NMR (CDCl₃, 300 MHz) δ 7.78 (s, 1H), 5.12 (s, 1H), 4.27 (m, 4H), 3.78 (s, 3H), 1.29 (t, 6H); MS m/z: M+1=303.

Using the procedure described in Example 1 for converting Compound 1a to Compound 1c and substituting 27b for 1b, and Compound 17a for Compound 1a, Compound 27b was converted to Compound 109 (4 mg, MS m/z: M+1=460) and Compound 121 (18 mg, MS m/z: M+1=532).

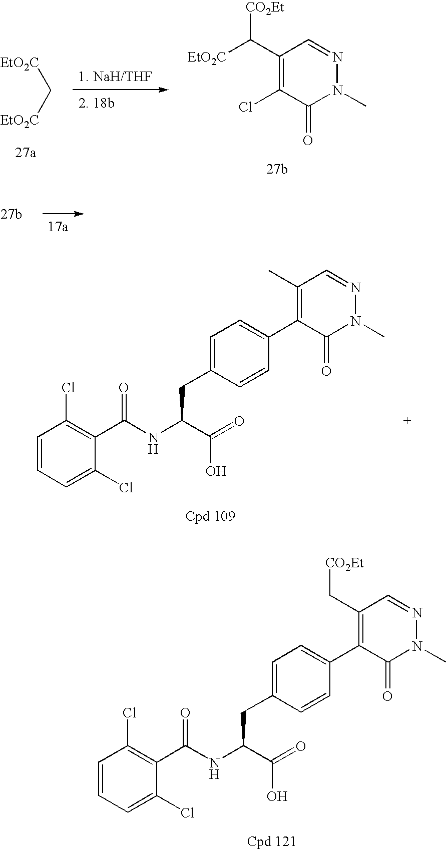

### EXAMPLE 28

### (S)-4-{4-[3-(2,6-Dichloro-benzoyl)-5-oxo-oxazol idin-4-ylmethyl]-phenyl}-5-methoxy-2-methyl-2H-pyridazin-3-one, Cpd 114

A mixture of Compound 1f (0.49 g, 1.0 mmol), paraformaldehyde (1.8 g, 60 mmol), and TsOH (19 mg, 0.1 mmol) in toluene (100 mL) was heated at 100° C. in an oil bath for 24 h. Paraformaldehyde formed on the top of the flask and in the condenser, so the glassware was scraped free of paraformaldehyde from time to time during the reaction. The toluene solution was concentrated and purified by HPLC to give Compound 114 as a white solid (0.25 g). MS m/z: M+1=488.

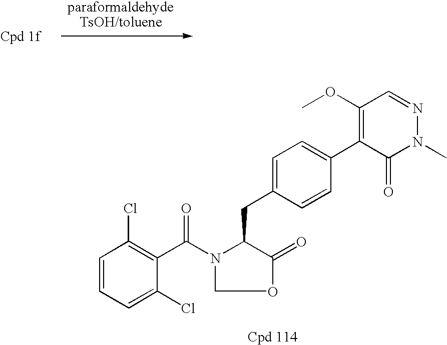

### EXAMPLE 29

### 3-[4-(5-Methoxy-2-methyl-3-oxo-2,3-dihydro-pyridazin-4-yl)-phenyl]-2(S)-{[2-(3-phenyl-propionyl)-2-aza-bicyclo[2.2.2]octane-3(S)-carbonyl]-amino}-propionic acid, Cpd 89

Using the procedure described in Example 11 for the conversion of Compound 1d to Compound 11b, substituting Compound 29a for Compound 11a, Compound 29b was prepared.

Using the procedure described in Example 12 for the conversion of Compound 11b to Compound 12a, Compound 29c was prepared.

To a solution of Compound 29c (0.17g, 0.37 mmol) in DCM (6 mL) was added TEA (66 μL, 0.46 mmol) followed by Compound 29d (66 μL, 0.44 mmol). The mixture was stirred at rt for 2h and then treated with dilute HCl solution. The DCM phase was washed with H₂O, NaHCO₃ (aq), and then again with H₂O. The organic phase was separated, dried over MgSO₄, and concentrated to give Compound 182 as a white solid (0.20 g). MS m/z: M+1=587.

Using the procedure of Example 12 for the conversion of Compound 12a to Compound 181, Compound 182 was converted to Compound 89. MS m/z: M+1=573.

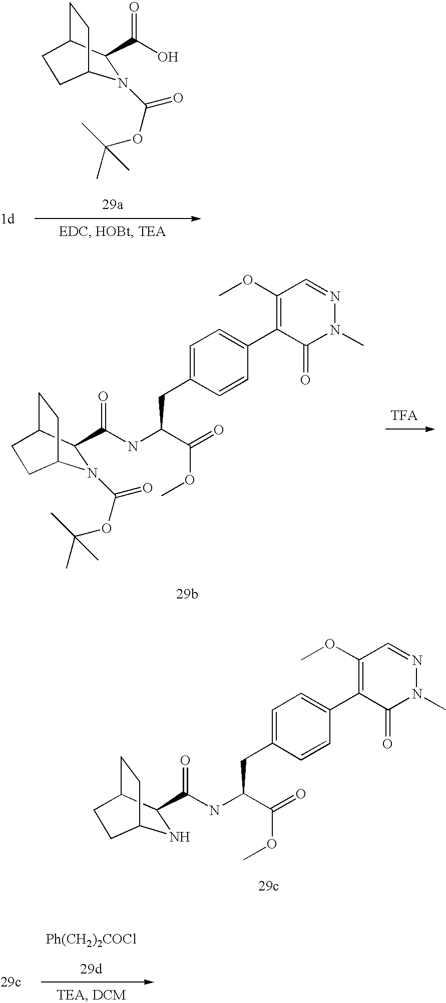

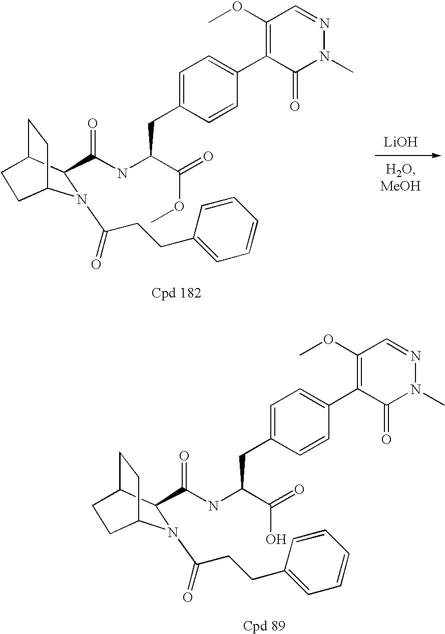

Other compounds of the present invention may be prepared by those skilled in the art by varying the starting materials, reagent(s) and conditions used. Using the procedure of Example 29, the following compounds were prepared without further purification:

| Cpd | MS (M + H⁺) | Cpd | MS (M + H⁺) |
|---|---|---|---|
| 122 | 563 | 125 | 527 |
| 126 | 541 | 139 | 580 |
| 174 | 541 | 113 | 565 |
| 100 | 539 | 115 | 563 |

### EXAMPLE 30

### 2-(2,6-Dichloro-benzoylamino)-3-[4-(5-methoxy-2-methyl-3-oxo-2,3-dihydro-pyridazin-4-yl)-phenyl]-propionic acid 2-hydroxy-ethyl ester, Cpd 97

To a solution of Compound 17 (0.94 g, 1.97 mmol) in DCM (6 mL) containing 1 mL of TEA was added BOP-Cl (590 mg, 2.33 mmol) followed by ethylene glycol (200 μL, 3.60 mmol). The mixture was stirred at rt overnight and then evaporated under reduced pressure at rt. The residue was subjected to column chromatography (silica, EtOAc) to yield a clear oil (650 mg). The oil was dissolved in a 2:1 MeOH-water mixture and lyophilized, providing Compound 97 as white powder: NMR (CD₃OD): δ 8.17 (s, 1H), 7.42-7.30 (m, 7H), 5.06 (dd, J=5.4 and 9.1 Hz), 4.41 (t, J=7.9 Hz, 2H), 4.21 (t, J=4.5 Hz, 2H), 3.93 (s, 3H), 3.77 (s, 3H), 3.72 (t, J=5.5 Hz, 2H), 3.58 (t, J=5.7 Hz, 2H), 1.35 (d, J=6.6 Hz, 1H); MS m/z M+H=520.

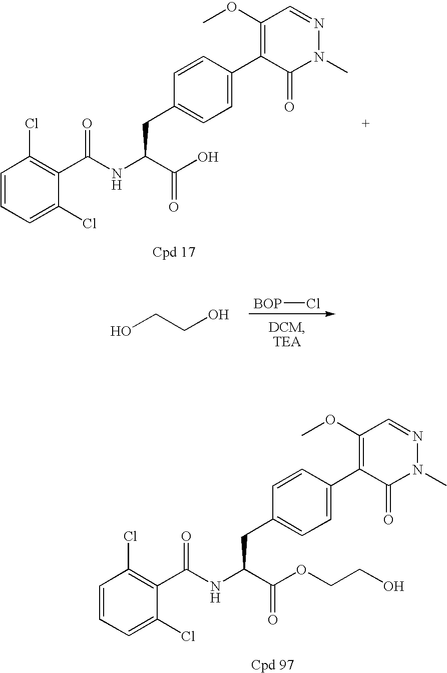

Other compounds of the present invention may be prepared by those skilled in the art by varying the starting materials, reagent(s) and conditions used. Using the procedure of Example 30, the following compounds were prepared without further purification:

Cpd 231: ¹H NMR (CD₃OD, 300 MHz) δ 8.22 (s, 1H), 7.40 (d, 2H, J=8.0 Hz), 7.29 (d, 2H, J=8.1 Hz), 6.98 (d, 1H, J=8.2 Hz), 4.37-4.45 (br m, 1H), 4.34 (t, 2H, J=5.7 Hz), 4.16-4.21 (br m, 2H), 3.96 (s, 3H), 3.94 (t, 2H, J=5.7 Hz), 3.69-3.74 (br m), 3.20 (dd, 1H, J=13.8, 5.4 Hz), 3.01 (dd, 1H, J=13.5, 8.8 Hz), 1.42 (s, 9H); MS: m/z 478 (M+H)⁺.

### Biological Experimental Examples

As demonstrated by biological studies described hereinafter, and shown in Table III, the compounds of the present invention are α4β1 and α4β7 integrin receptor antagonists useful in treating integrin mediated disorders including, but not limited to, inflammatory, autoimmune and cell-proliferative disorders.

### EXAMPLE 1

### Ramos Cell Adhesion Assay (α₄β₁ Mediated Adhesion/VCAM-1)

Immulon 96 well plates (Dynex) were coated with 100 μL recombinant hVCAM-1 at 4.0 μg/mL in 0.05 M NaCO₃ buffer pH 9.0 overnight at 4° C. (R&D Systems). Plates were washed 2 times in PBS with 1% BSA and blocked for 1 h @ room temperature in this buffer. PBS was removed and compounds to be tested (50 μL) were added at 2× concentration. Ramos cells, (50 μL at 2×10⁶/mL) labeled with 5 μM Calcein AM (Molecular Probes) for 1 h at 37° C., were added to each well and allowed to adhere for 1 h at room temperature. Plates were washed 4× in PBS+1% BSA and cells were lysed for 15 minutes in 100 μL of 1 M Tris pH 8.0 with 1% SDS. The plate was read at 485 nm excitation and 530 nm emission. Resulting data is shown in Table V.

### EXAMPLE 2

### α₄β₇-K562 Cell Adhesion Assay (α₄β₇ Mediated Adhesion/MAdCAM-1)

M2 anti-FLAG Antibody Coated 96-well plates (Sigma) were coated for 1 hour at 4° C. with 2-8 μl/well recombinant FLAG-hMAdCAM-1 contained in 100 μL of Dulbecco's PBS, pH 7.4, with 1% BSA and 1 mM Mn ⁺2 (PBS-BSA-Mn). Plates were washed once with PBS-BSA-Mn. Buffer was removed and compounds to be tested (50 μL) were added at 2× concentration. Stably transfected K562 cells expressing human α4β7 integrin, (50 μL at 2×10⁶/mL) that had been labeled with 100 μg/ml carboxymethyl fluorescein diacetate succinimidyl ester (CFDA-SE; Molecular Probes) for 15 min at 37° C. were added to each well and allowed to adhere for 1 h at room temperature. Plates were washed 4× in PBS-BSA-Mn and then cells were lysed for 2 minutes by addtion of 100 μL of PBS without Ca, Mg supplemented with 0.1 M NaOH. The plate was read on a 96-well fluorescent plate reader at 485 nm excitation and 530 nm emission. Resulting data is shown in Table V.

TABLE V

| | α4β1 | α4β7 |
|---|---|---|
| Cpd | IC₅₀ (μM) | IC₅₀ (μM) |
| *1 | >5 | nt |
| *2 | >5 | nt |
| 3 | >5 | 0.067 |
| 4 | 0.168 | 0.001 |
| 5 | 0.307 | 0.001 |
| 6 | 0.049 | 0.001 |
| 7 | 0.192 | 0.002 |
| 8 | 0.303 | 0.002 |
| 9 | 0.023 | 0.002 |
| 10 | 0.363 | 0.002 |
| 11 | 0.842 | 0.002 |
| 12 | 0.305 | 0.002 |
| 13 | 0.130 | 0.002 |
| 14 | 0.150 | 0.002 |
| 15 | 0.125 | 0.002 |
| 16 | 0.093 | 0.002 |
| 17 | 0.031 | 0.003 |
| 18 | >1 | 0.003 |
| 19 | 0.044 | 0.003 |
| 20 | 0.484 | 0.003 |
| 21 | 0.065 | 0.003 |
| 22 | 0.376 | 0.003 |
| 23 | 0.248 | 0.003 |
| 24 | 0.249 | 0.004 |
| 25 | 0.133 | 0.005 |
| 26 | 0.291 | 0.005 |
| 27 | 0.403 | 0.005 |
| 28 | 2.621 | 0.005 |
| 29 | >1 | 0.006 |
| 30 | 0.284 | 0.006 |
| 31 | 0.310 | 0.006 |
| 32 | 0.791 | 0.007 |
| 33 | 2.400 | 0.007 |
| *34 | 0.250 | 0.008 |
| 35 | 0.342 | 0.008 |
| 36 | 1.313 | 0.009 |
| 37 | 0.474 | 0.009 |
| 38 | >5 | 0.009 |
| 39 | 0.588 | 0.01 |
| 40 | >5 | 0.038 |
| 41 | 0.152 | 0.011 |
| 42 | 0.936 | 0.059 |
| 43 | 0.437 | 0.012 |
| 44 | 1.217 | 0.012 |
| 45 | >5 | 0.021 |
| 46 | 0.370 | 0.013 |
| 47 | 0.339 | 0.013 |
| 48 | 0.974 | 0.013 |
| 49 | 0.486 | 0.014 |
| 50 | 0.229 | 0.014 |
| 51 | 0.113 | 0.016 |
| 52 | 0.663 | 0.017 |
| 53 | 0.269 | 0.017 |
| 54 | 4.022 | 0.017 |
| 55 | 0.005 | 0.018 |
| 56 | 3.860 | 0.018 |
| 57 | 1.220 | 0.018 |
| 58 | >5 | 0.018 |
| 59 | 0.465 | 0.019 |
| 60 | 0.155 | 0.020 |
| 61 | 0.221 | 0.020 |
| 62 | 0.040 | 0.021 |
| 63 | 0.567 | 0.021 |
| 64 | 0.523 | 0.021 |
| 65 | 4.220 | 0.021 |
| 67 | 0.737 | 0.023 |
| 68 | 0.473 | 0.024 |
| 69 | >5 | 0.024 |
| 70 | >5 | 0.024 |
| 71 | 1.538 | 0.025 |
| 72 | 0.190 | 0.026 |
| 74 | 0.135 | 0.027 |
| 75 | >5 | 0.028 |
| 76 | 0.7295 | 0.035 |
| 77 | 0.24 | 0.036 |
| 78 | 5.611 | 0.036 |
| 79 | 0.511 | 0.036 |
| 80 | >5 | 0.228 |
| 81 | 0.987 | 0.039 |
| 82 | 2.693 | 0.039 |
| 83 | 1.016 | 0.041 |
| 84 | >5 | 0.041 |
| 85 | 0.794 | 0.042 |
| 86 | 0.212 | 0.044 |
| 87 | 0.2785 | 0.044 |
| 88 | 1.041 | 0.046 |
| 89 | 0.0397 | 0.047 |
| 90 | 4.5 | 0.048 |
| *91 | 2.38 | 0.049 |
| 92 | >5 | 0.052 |
| 93 | >5 | 0.052 |
| 94 | 1.321 | 0.053 |
| 95 | 0.48 | 0.0535 |
| 96 | 1.79 | 0.056 |
| *97 | 3.24 | 0.058 |
| 98 | 3.84 | 0.248 |
| 99 | 0.2645 | 0.06 |
| 100 | 0.272 | 0.06 |
| 101 | >5 | 0.061 |
| 102 | 1.0715 | 0.066 |
| 103 | 0.629 | 0.067 |
| 105 | 0.8325 | 0.069 |
| 106 | 2.097 | 0.069 |
| 107 | 0.3055 | 0.071 |
| 108 | 0.0027 | 0.072 |
| 109 | 3.2 | 0.078 |
| 110 | >5 | 0.08 |
| 111 | >5 | 0.082 |
| 112 | 0.289 | 0.085 |
| 113 | 0.044 | 0.088 |
| *114 | >5 | 0.093 |
| 115 | 0.0635 | 0.094 |
| 116 | 0.656 | 0.097 |
| 117 | 0.518 | 0.102 |
| *118 | 1.29 | 0.103 |
| *119 | 3.8 | 0.105 |
| 120 | >5 | 0.113 |
| 121 | >5 | 0.114 |
| 122 | 0.142 | 0.118 |
| 123 | >5 | 0.118 |
| 124 | >1 | 0.119 |
| 125 | 0.112 | 0.123 |
| 126 | 2.455 | 0.127 |
| 127 | >1 | 0.133 |
| 128 | 1.486 | 0.138 |
| 129 | >1 | 0.141 |
| 130 | >1 | 0.147 |
| 131 | >5 | 0.152 |
| 132 | >5 | 0.152 |
| 133 | 1.277 | 0.01 |
| 134 | >1 | 0.165 |
| 135 | >1 | 0.165 |
| 136 | 1.16 | 0.179 |
| 137 | 0.962 | 0.200 |
| 138 | 0.508 | 0.204 |
| 139 | 0.319 | 0.228 |
| 140 | 0.345 | 0.228 |
| 141 | >5 | 0.229 |
| 142 | >1 | 0.23 |
| 143 | >1 | 0.244 |
| 144 | 0.792 | 0.246 |
| 145 | >5 | 0.248 |
| 146 | 0.218 | 0.010 |
| 147 | >5 | 0.249 |
| 148 | >5 | 0.261 |
| 150 | 0.154 | 0.271 |
| *151 | >5 | 0.274 |
| 152 | >5 | 0.282 |
| 153 | 2.046 | 0.295 |
| 154 | >5 | 0.330 |
| 155 | >1 | 0.332 |
| 156 | >5 | 0.339 |
| 157 | >5 | 0.353 |
| 158 | >1 | 0.356 |
| 159 | >5 | 0.363 |
| 160 | 0.611 | 0.364 |
| 161 | 0.471 | 0.4 |
| 162 | >5 | 0.408 |
| 163 | >5 | 0.429 |
| 164 | >5 | 0.457 |
| 165 | >5 | 0.471 |
| 166 | >5 | 0.478 |
| 167 | >5 | 0.48 |
| 168 | >1 | 0.49 |
| 169 | 0.370 | 0.500 |
| 170 | 2.81 | 0.514 |
| 171 | >5 | 0.536 |
| 172 | >5 | 0.570 |
| 173 | >5 | 0.575 |
| 174 | 0.604 | 0.585 |
| *175 | >5 | 0.591 |
| 176 | >5 | 0.614 |
| 177 | >5 | 0.163 |
| 178 | >5 | 0.706 |
| 179 | >5 | 0.721 |
| 180 | >5 | 0.731 |
| 181 | >5 | 0.750 |
| *182 | >1 | 0.785 |
| 183 | >5 | 0.805 |
| 184 | >5 | 0.813 |
| 185 | >5 | 0.817 |
| 186 | 1.530 | 0.842 |
| 187 | >5 | 0.865 |
| 188 | >5 | 0.922 |
| 189 | >5 | 0.937 |
| 190 | >5 | 0.979 |
| 191 | >5 | 0.999 |
| 192 | >5 | 1.025 |
| 193 | >5 | 1.164 |
| 194 | >5 | 1.220 |
| 195 | >5 | 1.280 |
| 196 | >5 | 1.290 |
| 197 | >1 | 1.320 |
| 198 | >5 | 1.340 |
| *199 | >5 | 1.370 |
| 200 | >5 | 1.410 |
| 201 | >5 | 1.444 |
| 202 | >5 | 1.480 |
| 203 | >5 | 1.776 |
| 204 | >5 | 1.869 |
| 205 | >5 | 2.32 |
| 206 | 3.300 | 2.55 |
| *207 | >5 | 2.69 |
| 208 | >5 | 2.760 |
| 209 | >5 | 3.137 |
| 210 | >5 | 4.648 |
| 211 | >5 | 0.529 |
| *212 | >5 | >5 |
| 213 | 2.880 | 0.264 |
| 214 | 1.066 | 0.023 |
| 215 | >5 | 0.305 |
| 216 | >5 | 0.101 |
| 217 | >5 | 0.640 |
| 218 | >5 | 0.239 |
| 219 | >5 | 0.852 |
| 220 | >5 | 0.263 |
| 221 | >5 | 0.380 |
| 222 | >5 | 0.401 |
| 223 | >5 | 0.115 |
| 224 | >5 | 0.014 |
| 225 | 0.472 | 0.013 |
| 226 | 0.925 | 0.031 |
| 227 | >5 | 0.219 |
| 228 | 0.535 | 0.006 |
| 229 | >5 | 0.089 |
| 230 | NT | 0.092 |
| *231 | NT | NT |
| *232 | >5 | >5 |

*indicates a prodrug

### EXAMPLE 3

### In Vivo Model for Leukocytosis

Leukocytosis is the increase in circulating white blood cells (leukocytes). This can be brought about by preventing leukocyte binding to counter-receptor adhesion molecules expressed on high endothelial venules. This cell adhesion occurs between immunoglobulin superfamily molecules and integrins. Relevant examples of these paired interactions include Intracellular Adhesion Molecule-1 and AlphaL Beta2 integrin, Vascular Cell Adhesion Molecule-1 and α4β1 integrin, and Mucosal Addressin Cell Adhesion Molecule-1 and α4β7 integrin, respectively.

In this model, a compound that antagonizes these leukocyte-endothelial interactions will cause an increase in circulating leukocytes, defined as leukocytosis, as measured at 1-1.5 h post-administration. This leukocytosis is indicative that normal lymphocyte or leukocyte emigration from the peripheral circulation was prevented. Similar emigration of cells out of the circulation into inflamed tissues is responsible for the progression and maintainance of the inflammatory state. Leukocytosis is an indication that lymphocyte and leukocyte extravasation is prevented, and is predictive of general anti-inflammatory activity.

Procedure

One week prior to being tested, 7-10 week old female Balb/c mice, n=8 per group, were bled and randomized according to leukocyte counts. One week later, the mice were adminstered test compound orally or subcutaneously and then bled 1-1.5 h after drug administration, approximately 1 h after the peak blood concentration of the compound occurred. Whole blood, 250-350 microliters, was collected from each mouse into potassium-EDTA serum collection tubes (Becton-Dickenson) and mixed to prevent clotting.

Cell counts and differential counts on the whole blood preparation were performed using an Advia 120 Hematology System (Bayer Diagnostics). Cell counts as total leukocytes and as total lympohcytes were made and compared to counts made from mice dosed with vehicle only. Data were reported as percent of vehicle control for lymphocyte counts and total leukocyte counts.

Statistical analyses were performed using ANOVA with Dunnet's multiple comparison test. Resulting data is shown in Table VI.

TABLE VI

| | | Lymphoycte Counts | | | Total Leukocyte Count | | |
|---|---|---|---|---|---|---|---|
| | | % of Vehicle Control | | | % of Vehicle Control | | |
| Cpd | Rte | 3 mg/kg | 10 mg/kg | 30 mg/kg | 3 mg/kg | 10 mg/kg | 30 mg/kg |
| 17 | sc | 145.3 | 191.7 | 220.2 | 143.3 | 198.7 | 264.5 |
| | | p < 0.05 | p < 0.05 | p < 0.05 | p < 0.05 | p < 0.05 | p < 0.05 |
| 120 | sc | 104.3 | 112.8 | 80.0 | 91.8 | 101.7 | 78.0 |
| 231 | po | 114.5 | 94.0 | 88.7 | 110.7 | 98.0 | 86.9 |

p < 0.05 = significant increase vs. vehicle-treated control, ANOVA with Dunnets multiple comparisons test

### EXAMPLE 4

### Phorbol 12-Myristate 13-Acetate (PMA)-Induced Inflammation in Mouse Ear Skin and Measurement of Eosinophil Peroxidase

Phorbol 12-myristate 13-acetate (PMA) when applied to skin, generates a vigorous recruitment of immune cells to the site of application. Over a 24 hour period, there is accumulation of fluid and cells to the inflamed site, and thus is a general indicator of an inflammatory response. Among the recruited cells are eosinophils and neutrophils. Eosinophils can migrate into an inflamed or infected tissue via alpha 4 beta 1 integrin interactions with vascular cell adesion molecule-1 (VCAM-1) counter-receptors on vascular endothelial cells, and via alpha 4 beta 7 integrin to mucosal addressin cellular adhesion molecule on vascular endothelial cells in the gastrointestinal tract and mesenteric system. The recruited esoinophils can be quantified by measuring the presence of eosinophil peroxidase in a sample of the homogenized tissue. Those that are recruited to the inflamed site in the ear do so via integrin-Ig superfamily receptor pairs that notably include alpha 4 beta 1 integrin—VCAM-1 interactions.

### Induction of Ear Inflammation and Treatment of Animals

Female BALB/C mice are ordered at 6 weeks of age and 16-18 grams from Charles River were used between 6-10 weeks of age. The animals were randomly assigned to groups of 10 (5/box) and housed in groups in plastic cages in a room with 12 h light-dark cycle and controlled temperature and humidity. They received food and water ad libitum.

Phorbol 12-myristate 13-acetate (PMA) was dissolved as 5 mg per mL stock in dimethyl sulfoxide (DMSO) and stored frozen as 20 microliter aliquots. For application to mouse ears, each aliquot was diluted in 2 mL with acetone.

The right ear of each mouse was treated topically with 20 microliters of acetone solution (10 microliters to each side of the ear) containing either 1 microgram of phorbol 12-myristate 13-acetate (PMA) or acetone alone.

Drugs that were tested orally were administered at −1 and +3 hours relative to PMA application.

Estimation of Ear Tissue Eosinophil Content By Assay of Eosinophil Peroxidase.

Mice were sacrificed 24 h after PMA application. The right ear was punched with a 6 mm tissue punch and the tissue was placed in a tube on dry ice and kept frozen until extraction.

Methods

### Substrate Buffer Preparation

One tablet of phosphate citrate buffer was dissolved with urea hydrogen peroxide in 100 ml of water in which one tablet containing 60 mg of o-phenylenediaminedihydrochloride was added.

### Eosinophil Peroxidase Extraction

Ear tissue samples were homogenized in 2 ml of HTAB for 15 sec at speed 5.5 with a Polytron (large head) (Brinkman Instruments). The homogenate was stored at −20° C. until assayed.

### Eosinophil Peroxidase Assay

On the day of eosinophil peroxidase measurements, the ear tissue homogenates were heated to 60° C. for 2 h in a waterbath to guarantee the maximal recovery of eosinophil peroxidase activity.

After heating, samples were transferred into a 2 mL conical polypropylene microcentrifuge tube and spun for 10 min at 10,000×g in a microcentrifuge to clear debris.

Samples were typically tested at either a 1:2 or 1:4 dilution made with HTAB. A 100 μL portion of sample was pipetted into a 96-well microtiter plate (Costar no. 3595) followed by addition of 100 μL of substrate buffer. After 10 minutes of incubation at room temperature the reaction was stopped by adding 50 microliters of 4N H₂SO₄. Absorbance was read at 490 nm for the specific with subtraction of a 650 nm noise signal using a Thermomax 96-well spectrophotmetric plate reader (Molecular Devices).

Analysis was made using ANOVA on EXCEL and determining significance with Dunnett's Significant Difference compared to normal controls who received only an acetone application to the ear.

The inhibition of PMA-induced ear edema was measured by eosinophil peroxidase levels in ear punches. The ear punches were taken 24 h after PMA application to ear. Compounds were administered in 2 doses that equally divided the total dose. Administration was conducted 1 h before and 3 h after PMA application. Statistical significance was ascertained by ANOVA using Dunnet's multiple comparison's test. Resulting data is shown in Table VII.

TABLE VII

| | | | | | | p-value vs. |
|---|---|---|---|---|---|---|
| | Total | | | % Inh. of | | vehicle |
| | Dose | | Dosing | Eosinophil | | treated with |
| Cpd | (mg/kg) | Rte | Regimen | Peroxidase | SE | PMA control |
| dexa | 5 | po | bid | 109.0 | 2.8 | <0.05 |
| methasone | | | | | | |
| 89 | 40 | po | bid | 43.5 | 4.4 | ns |
| 113 | 40 | po | bid | | 11.8 | ns |
| dexa | 5 | po | bid | 83.6 | 4.0 | <0.05 |
| methasone | | | | | | |
| 17 | 40 | po | bid | 34.9 | 7.2 | ns |
| 151 | 40 | po | bid | 26.5 | 4.6 | ns |

### EXAMPLE 5

Intraperitoneal Delayed Type Hypersensitivity (IP-DTH) Response. A Method for Analyzing Effects of Integrin Antagonists In Vivo

Integrin antagonists are meant to interfere with the binding or adhesion of immune cells, such as lymphocytes, monocytes and eosinophils that bear integrin receptors to counter-receptors that exist on endothelial cells in the vasculature. Among those integrin-bearing cells, cells that are positive for alpha 4 beta 7 integrin (found in the mesenteric system and in the gut), would comprise many of the cells recruited to a peritoneal antigen challenge. One can maximize the number of alpha 4 beta 7 integrin-positive cells recruited by inducing an intraperitoneal delayed type hypersensitivity response to antigen that will recruite antigen-responsive cells from the mesenteric lymph nodes. An inhibitor of alpha 4 beta 7 integrin should prevent the recruitment of these cells to the site of antigen challenge. Alpha 4 beta 7 integrin-positive cells are considered to be gut-homing, and are found in greater abundance in inflamed tissues of the GI tract and pancrea.

The antigenic challenge will induce a delayed type hypersensitivity response. In this model, animals were primed with antigen, then 7 days later were challenged intraperitoneally with the same antigen. During the ensuing 24-48 h, cells that were primed to recognize this antigen should be recruited to the challenge site. If the site is the peritoneal cavity, the recruited cells can be obtained by ravaging the cavity with a physiological buffer and collecting the lavage fluid.

The contribution of alpha 4 beta 7 integrin positive cells to the peritonal cavity cell population was ascertained by using flow cytometry to evaluate their relative percent in this population.

Method

The mice were primed via intraperitoneal administration with 25 micrograms ovalbumin in a physiological buffer that may or may not contain alum as an adjuvant.

After 7 days, the mice were challenged with 25 micrograms ovalbumin via intraperitoneal administration.

Compounds were administered either orally (po), or subcutaneously (sc), either once daily or twice daily, for 2 days, starting on the the day of antigen challenge.

Forty eight hours after antigen challenge, the elicited cells in the peritoneal cavity were harvested by lavaging the cavity in physiological saline or phosphate buffered saline, with calcium and magnesium salts.

The cells were washed into Staining Buffer consisting of phosphate buffered saline, 1% bovine serum albumin and 0.1% sodium azide, and resuspended to 2×10e7 cells/ml. A portion of 1×10e6 cells was deposited into a 96-well V-bottom plate for staining.

The sample of 1×10e6 cells was stained with fluorochrome-coupled antibody to alpha 4 beta 7 integrin or a primary antibody to alpha 4 beta 7 integrin followed by a secondary fluorochrome-coupled antibody. Each staining step was carried out at 4° C. for 30 to 45 min with gentle shaking, followed by 4 washes with Staining Buffer at 4° C. The cells were resuspended in 200 microliters of 1% paraformaldehyde in phosphate buffered saline. The cells were then transferred to test tubes and maintained at 4° C. until analyzed by flow cytometry to determine numbers of alpha4 beta7-postive cells.

A Becton-Dickenson FACSort (B-D instruments) was used for these studies.

Comparisons were made between numbers of alpha 4 beta 7-positive cells in samples taken from antigen-treated animals and numbers of alpha 4 beta 7-positive cells taken from antigen-treated animals administered experimental compounds. Resultant data is presented in Table VII.

TABLE VII

| | DOSE | | Dose | % Decrease in |
|---|---|---|---|---|
| Cpd | (mg/kg) | Route | Regimen | recruited α4β7 + cells |
| 17 | 30 | sc | bid × 2 | 26.5 |
| 17 | 30 | sc | bid × 2 | 46.6 |
| 17 | 30 | sc | bid × 2 | 53.2 |
| 11 | 30 | sc | bid × 2 | 70.5 |
| 5 | 30 | sc | bid × 2 | 53.4 |
| 46 | 30 | sc | bid × 2 | 56.6 |
| 18 | 30 | sc | bid × 2 | 52.8 |
| 17 | 30 | sc | bid × 2 | 54.6 |
| 15 | 30 | sc | bid × 2 | 10.4 |
| 10 | 30 | sc | bid × 2 | 56.3 |
| 26 | 30 | sc | bid × 2 | 0 |
| 61 | 30 | sc | bid × 2 | 37.4 |
| 17 | 30 | sc | bid × 2 | 35.1 |
| 17 | 30 | sc | bid × 2 | 15.4 |
| 35 | 30 | sc | bid × 2 | 49.8 |
| 30 | 30 | sc | bid × 2 | 37.4 |
| 120 | 30 | po | bid × 2 | 25.4 |
| 232 | 30 | po | bid × 2 | 47.76 |
| 199 | 30 | po | bid × 2 | 16.04 |
| 207 | 30 | po | bid × 2 | 25.6 |
| 59** | 30 | po | bid × 2 | −39.8 |

Mean Value ± SE for Cpd 17 treatments, 30 mg/kg, sc, bid, ×2 (n = 6): 38.6 ± 7% Decrease
**Increased blood in peritoneum: not valid

### EXAMPLE 6

### In Vivo Model for Colitis: Dextran Sulfate Sodium (DSS) Induced Colitis

Inflammatory bowel diseases such as ulcerative colitis and Crohn's disease are characterized by diminished intestinal barrier function, apparent inflammatory damage that may include erosive loss of intestinal mucosa, and inflammatory infiltrates in the mucosa and submucosa.

Chemically induced models of experimental of colitis are used to mimic various aspects of these diseases. Among the many possible chemicals used are dextran sulfate sodium (DSS) and trinitrobenzene sulfonic acid (TNBS). The dextran sulfate sodium model of experimental colitis is characterized by a shrinkage of the colon's length, macrosopic inflammatory damage, diarrhea, a discontinuous pattern of mucosal epithelial damage in the distal colon with infiltration of inflammatory cells that include macrophages and neutrophils into the mucosa and submucosa (Blumberg, R. S., Saubermann, L. J., and Strober, W. Animal models of mucosal inflammation and their relation to human inflammatory bowel disease. Current Opinion in Immunology, 11: 648-656, 1999; Okayasu, I., Hatakeyama, S., Yamada, M., Ohkusa, T., Inagaki, Y., and Nakaya, R. A novel method of induction of reliable experimental acute and chronic colitis in mice. Gastroenterology, 98: 694-702, 1990; Cooper, H. S., Murthy, S. N. S., Shah, R. S., and Sedergran, D. J. Clinicopathologic study of dextran sulfate sodium experimental murine colitis. Lab Invest., 69: 238-249, 1993; Egger, B., Bajaj-Elliott, M., MacDonald, T. T., Inglin, R., Eysselein, V. E., and Buchler, M. W. Characterization of acute murine dextran sodium sulphate colitis: Cytokine profile and dose dependency. Digestion, 62: 240-248, 2000; Stevceva, L., Pavli, P., Husband, A. J., and Doe, W. F. The inflammatory infiltrate in the acute stage of the dextran sulphate sodium induced colitis: B cell response differs depending on the percentage of DSS used to induce it. BMC Clinical Pathology, 1: 3-13, 2001; and Diaz-Granados, Howe, K., Lu, J, and McKay, D. M. Dextran sulfate sodium-induced colonic histopathology, but not altered epithelial ion transport, is reduced by inhibition of phosphodiesterase activity. Amer. J. Pathology, 156: 2169-2177, 2000).

Methodology

Balb/c female mice and C57Black/6 mice were used in these studies. The Baslb/c mice were provided with a solution of tap water containing 5% DSS (ICN chemicals) ad libitum over a 7-day period. When C57Black/6 mice were used, a solution of tap water containing 4% DSS was used. During the ensuing 7-day period, test animals were administered a preparation of an experimental compound. This material may be administered orally or intraperitoneally or subcutaneously, once or twice daily. At the end of this period, the animals were euthanized and their colons were collected for further analysis. Among the parameters analyzed were the length of the colon starting from the anus to the top of the cecum, the consistency of any stools found within the colon, and the gross macroscopic appearance of the colon. The distal colon between the 1^{st} and the 4^{th} centimeter was removed and placed in 10% neutral buffered formalin for later histological analysis.

For the following parameters, colon length, stool consistency and appearance, and macroscopic damage a scoring system is used to describe the changes. The 3 scores for each animal are added to provide a Total Macroscopic Score. Thus,

Stool Score: 0=normal (well-formed fecal pellets); 1=loosely-shaped moist pellets; 2=amorphous, moist, sticky pellets; 3=severe diarrhea.

Colon Damage Score: 0=no inflammation; 1=reddening mild inflammation; 2=moderate inflammation or more widely distributed; 3=severe inflammation and/or extensively distributed.

Colon Length Score: 0=<5% shortening; 1=5-14% shortening; 2=15-24% shortening; 3=25-35% shortening; 4=>35% shortening.

Histological analyses of tissues consisted of staining paraffin-embedded tissue sections with hematoxylin-eosin dye. Epithelial damage scores were determined as the fraction of the tissue section showing damaged epithelium. Scores were determined as follows: 0=no damage; 1=<⅓ damaged, 2=⅓ to <⅔ damaged, 3=>⅔ damaged. Resulting data is shown in Table VIII and Table IX.

Statistical analysis performed in Graphpad Prism 4.0 using ANOVA with Dunnet's or Bonferroni's multiple comparison's test.

TABLE VIII

| | | | | % | | | | |
|---|---|---|---|---|---|---|---|---|
| | | | | Inhibition | | | | |
| | | | | of Total | | | | |
| | | | Dose | Macro | | | | |
| | DOSE | | Regi- | scopic | | p | | mouse |
| Cpd | (mg/kg) | Rte | men | Score | SE | value | n | strain |
| 17 | 3 | sc | bid | 32.95 | 13.8 | p > 0.05 | 9 | balb/c |
| | 10 | sc | bid | 30.6 | 8.5 | p > 0.05 | 8 | |
| | 30 | sc | bid | −32.8 | 6.4 | p > 0.05 | 8 | |
| 17 | 10 | sc | bid | −7.2 | 10.9 | p > 0.05 | 10 | C57 |
| | | | | | | | | Black/6 |
| | 30 | sc | bid | 11.2 | 12.6 | p > 0.05 | 10 | |
| 231 | 30 | po | bid | 43.8 | 13.4 | p < 0.05 | 9 | balb/c |
| | 60 | po | bid | 47.7 | 11 | p < 0.01 | 18 | |
| 231 | 30 | po | bid | 13.4 | 5.5 | p > 0.05 | 20 | C57 |
| | | | | | | | | Black/6 |
| | 60 | po | bid | 23.4 | 5.6 | p > 0.05 | 9 | |

TABLE IX

| | | | | % Inhibi- | | | | |
|---|---|---|---|---|---|---|---|---|
| | | | | tion of | | | | |
| | | | Dose | Epithelial | | | | |
| | DOSE | | Regi- | Damage | | | | mouse |
| Cpd | (mg/kg) | Rte | men | Score | SE | p-value | | strain |
| 17 | 3 | sc | bid | 61.11 | 25.72 | p > 0.05 | 9 | balb/c |
| | 10 | sc | bid | 56.3 | 28.6 | p > 0.05 | 8 | |
| | 30 | sc | bid | 78.1 | 21.9 | p > 0.05 | 8 | |
| 17 | 10 | sc | bid | 33.3 | 11.1 | p > 0.05 | 10 | C57 |
| | | | | | | | | Black/6 |
| | 30 | sc | bid | 50 | 13.6 | p < 0.05 | 10 | |
| 231 | 30 | po | bid | nd | nd | nd | nd | balb/c |
| | 60 | po | bid | 36.5 | 17.2 | p > 0.05 | 18 | |
| 231 | 30 | po | bid | 23.91 | 12.66 | p > 0.05 | 20 | C57 |
| | | | | | | | | Black/6 |
| | 60 | po | bid | 53.9 | 7.5 | p < 0.01 | 9 | |

Note:
nd = no data

### EXAMPLE 7

### In Vivo Model: Trinitrobenzenesulfonic acid (TNBS) Induced Colitis

The TNBS model of experimental colitis (Bobin-Dubigeon, C., Collin, X., Grimaud, N., Robert, J-M., Guillaume Le Baut, G., and Petit, J-Y. Effects of tumour necrosis factor-a synthesis inhibitors on rat trinitrobenzene sulphonic acid-induced chronic colitis. Eur. J. Pharmacology, 431: 103-110, 2001), is characterized by shrinkage of the colon, intraperitoneal serosal adhesions, severe wounding and inflammatory damage, diarrhea, a continuous pattern of mucosal epithelial damage in the distal colon with infiltration of inflammatory cells. These symptomatic signs in the above—mentioned models are similar to what occur in human colitis.

Male Wistar rats (200-250 g) are inoculated with 500 microliters of a solution of 10 to 20 mg of TNBS in 30% ethanol delivered intracolonically via catheter or ball-tipped gavage needle to the 8^{th} cm from the anus. When Balb/c female mice (8-12 weeks of age) were used, the inoculation volume was 50 microliters containing 2-3 mg of TNBS in 30% ethanol delivered intracolonically via catheter or ball-tipped gavage needle to the 4^{th} cm from the anus. During the ensuing 7 days, test animals were administered a preparation of an experimental compound. This material may be administered orally, subcutaenously or intraperitoneally, once or twice daily. At the end of this period, the animals were euthanized and their colons were collected for further analysis. Among the parameters analyzed were the length of the colon starting from the anus to the top of the cecum, the weight of the colon, the consistency of any stools found within the colon, the presence or absence of intraperitoneal adhesions on the serosal surfacr of the intestin, and the gross macroscopic appearance of the colon. The latter is scored for length and severity of inlfammatory damage using a 10 point score. In rats, the distal colon between the 5^{th} and the 8^{th} centimeter is dissected and placed in 10% neutral buffered formalin for later histological analysis. In mice, the 1^{st} to the 4^{th} cm was collected for histological analyses.

For the following parameters—colon length, colon weight, stool consistency and appearance and macroscopic damage—a scoring system was used to describe the changes. The four scores for each animal were added to provide a Total Score.

Stool Score: 0=normal (well-formed fecal pellets); 1=loosely-shaped moist pellets; 2=amorphous, moist, sticky pellets; 3=bloody diarrhea. For the presence of blood in stool, one point was added to scores <3.

Colon Damage Score: 0=no inflammation; 1=focal hyperemia; 2=ulceration without hyperemia at one site; 3=ulceration and hyperemia at one site; 4=2 or more sites of ulceration and hyperemia; 5=multiple sites of damage extending to >1 cm; 6-10=multiple sites of damage extending to >2 cm; one point was added for each additional cm of tissue involvement.

Colon Weight Score: 0=<5% weight gain; 1=5-14% weight gain; 2=15-24% weight gain; 3=25-35% weight gain; 4=>35% weight gain.

Colon Length Score: 0=<5% shortening; 1=5-14% shortening; 2=15-24% shortening; 3=25-35% shortening; 4=>35% shortening.

Histological analyses of tissues consisted of staining paraffin-embedded tissue sections with hematoxylin-eosin dye. Epithelial damage scores were determined as the fraction of the tissue section showing damaged epithelium. Scores were determined as follows: 0=no damage; 1=<⅓ damaged, 2=⅓ to <⅔ damaged, 3=>⅔ damaged.

Resulting data is shown in Table X and Table XI. Statistic analyses for these experiments performed with Graphpad Prism 4.0, using ANOVA, with Dunnets or Bonferroni's multiple comparisons test.

TABLE X

| | | | | % Inhi- | | | | |
|---|---|---|---|---|---|---|---|---|
| | | | | bition | | | | |
| | | | | of Total | | | | |
| | | | Dose | Macro | | | | |
| | DOSE | | Regi- | scopic | | p | | |
| Cpd | (mg/kg) | Rte | men | Score | SE | value | n | Species |
| 17 | 10 | sc | bid | −1.638 | 7.01 | p > 0.05 | 9 | rat |
| | 30 | sc | bid | −4.117 | 6.824 | p > 0.05 | 32 | |
| 17 | 10 | sc | bid | 21.30 | 14.5 | p > 0.05 | 10 | mouse |
| | 30 | sc | bid | 25.5 | 14.1 | p > 0.05 | 10 | |
| | 60 | sc | bid | −17 | 24.1 | p > 0.05 | 10 | |
| 231 | 30 | po | bid | 19.33 | 9.877 | p > 0.05 | 24 | rat |
| | 60 | po | bid | 5.455 | 10.46 | p > 0.05 | 11 | |
| 2 | 30 | po | bid | −0.885 | 14.46 | p > 0.05 | 12 | rat |

TABLE XI

| | | | | % | | | | |
|---|---|---|---|---|---|---|---|---|
| | | | | Inhibition | | | | |
| | | | | of | | | | |
| | | | Dose | Epithelial | | | | |
| | DOSE | | Regi- | Damage | | p | | |
| Cpd | (mg/kg) | Rte | men | Score | SE | value | n | Species |
| 17 | 10 | sc | bid | −5.822 | 13.23 | p > 0.05 | 9 | rat |
| | 30 | sc | bid | 8.5 | 11.76 | p > 0.05 | 31 | rat |
| 231 | 30 | po | bid | 24.69 | 17.99 | p > 0.05 | 23 | rat |
| | 60 | po | bid | 38.64 | 16.98 | p > 0.05 | 11 | |
| 2 | 30 | po | bid | 10.25 | 18.54 | p > 0.05 | 12 | rat |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A compound of Formula (I) 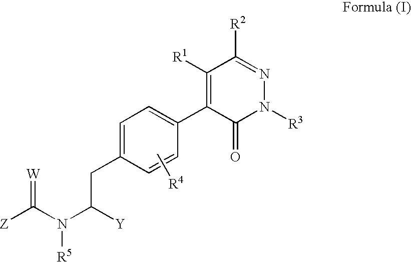
wherein
R¹ is a substituent independently selected from the group consisting of hydrogen, C₁₋₆alkyl, C₁₋₆alkoxy, aryl, heteroaryl, heterocyclyl, benzo fused heterocyclyl, benzo fused cycloalkyl, heteroaryl fused heterocyclyl, heteroaryl fused cycloalkyl, aryloxy, heteroaryloxy, heterocyclyloxy, cycloalkyloxy, —NR¹⁰R²⁰, halogen, hydroxy, and —S(C₁₋₆)alkyl; wherein the C₁₋₆alkoxy is optionally substituted with one to four substituents independently selected from R^{a};
wherein R^{a} is independently selected from the group consisting of hydroxy(C₁₋₆)alkoxy, aryl, heteroaryl, heterocyclyl, cycloalkyl, (C₁₋₆)alkoxycarbonyl, carboxy, amino, alkylamino, dialkylamino, one to three halogen atoms, and hydroxy;
wherein R¹⁰ and R²⁰ are independently selected from the group consisting of hydrogen, C₁₋₆alkyl, allyl, halogenated C₁₋₆alkyl, hydroxy, hydroxy(C₁₋₄)alkyl, aryl, aryl(C₁₋₄)alkyl, and cycloalkyl; additionally, R¹⁰ and R²⁰ are optionally taken together with the atoms to which they are attached to form a five to seven membered monocyclic ring;
wherein the aryl and aryloxy substituents of R¹ are optionally substituted with a substituent independently selected from the group consisting of C₁₋₆alkyl, hydroxy(C₁₋₆)alkyl, aryl(C₁₋₆)alkyl,
C₁₋₆alkoxy, aryl, heteroaryl, C₁₋₆alkoxycarbonyl, aryl(C₁₋₆)alkoxycarbonyl, C₁₋₆alkylcarbonyl, aminocarbonyl, alkylaminocarbonyl, dialkylaminocarbonyl, hydroxy, cyano, nitro, —SO₂(C₁₋₃)alkyl, —SO₂aryl, —SO₂heteroaryl, trifluoromethyl, trifluoromethoxy, and halogen;
and wherein the heteroaryl and heterocyclyl substituents of R¹ are optionally substituted with a substituent independently selected from the group consisting of one to three C₁₋₆alkyl substituents, C₁₋₆alkoxy, aryl, heteroaryl, one to three halogen atoms, and hydroxy;
R² is a substituent independently selected from the group consisting of hydrogen, C₁₋₆alkyl, C₁₋₆alkoxy, C₂₋₆alkenyloxy, hydroxy, amino, alkylamino, dialkylamino, and halogen;
wherein R¹ and R² are optionally taken together with the atoms to which they are attached to form a five to seven membered carbocyclic or heterocyclic ring;
R³ is a substituent independently selected from the group consisting of hydrogen, C₁₋₆alkyl, C₂₋₆alkenyl, C₂₋₆alkynyl, aryl, heteroaryl, heterocyclyl, and cycloalkyl; wherein alkyl, alkenyl, and alkynyl are optionally substituted with a substituent independently selected from aminocarbonyl, alkylaminocarbonyl, dialkylaminocarbonyl, aryl, heteroaryl, heterocyclyl, cycloalkyl, carboxy, one to three halogen atoms, hydroxy, or —C(═O)C₁₋₆alkyl;
R⁴ is a substituent independently selected from the group consisting of hydrogen, fluorine, chlorine, and methyl;
R⁵ is hydrogen or C₁₋₃alkyl, provided that R⁵ is C₁₋₃alkyl only when taken with Y and the atoms to which R⁵ and Y are attached to form a five to seven membered heterocycle;
Y is independently selected from the group consisting of hydroxymethyl, —C(═O)NH₂, —C(═O)NH(OH), —C(═O)NH(C₁₋₆alkyl), —C(═O)NH(hydroxy(C₁₋₆)alkyl), —C(═O)N(C₁₋₆alkyl)₂, —C(═O)NHSO₂(C₁₋₄)alkyl, carboxy, tetrazolyl, and —C(═O)C₁₋₆alkoxy; wherein said alkoxy is optionally substituted with one to two substituents independently selected from hydroxy, —NR³⁰R⁴⁰, heterocyclyl, heteroaryl, halogen, or —OCH₂CH₂OCH₃;
wherein R³⁰ and R⁴⁰ are independently selected from the group consisting of hydrogen, C₁₋₆alkyl, hydroxy, and hydroxy(C₁₋₄)alkyl, and said R³⁰ and R⁴⁰ are optionally taken together with the atoms to which they are attached to form a five to seven membered monocyclic ring;
W is O or S;
Z is selected from the group consisting of hydrogen, C₁₋₆alkyl, C₁₋₆alkenyl, C₁₋₆alkynyl, C₁₋₆alkoxy, aryl, heteroaryl, cycloalkyl, heterocyclyl, cycloalkyloxy, polycycloalkyloxy, and aza-bridged polycycyl wherein aza-bridged polycycyl is optionally substituted with R^{d};
wherein alkyl and alkoxy are optionally substituted with one to three substituents independently selected from the group consisting of aryl, aryl(C₁₋₄)alkoxy, heteroaryl optionally substituted with one to three C₁₋₂alkyl substitutents or —C(═O)aryl, hydroxy, —C(═O)C₁₋₆alkyl, —NH₂, —NH(C₁₋₆alkyl), —N(C₁₋₆alkyl)₂, —NH(cycloalkyl) wherein said cycloalkyl is optionally spirofused to a heterocyclyl, —NHC(═O)aryl(C₁₋₄)alkoxy, —N(C₁₋₆alkyl)C(═O)aryl(C₁₋₄)alkoxy, —NHC(═O)heteroaryl(C₁₋₄)alkyl, —N(C₁₋₆alkyl)C(═O)heteroaryl(C₁₋₄)alkyl, —NHC(═O)aryl(C₁₋₄)alkyl, —N(C₁₋₆alkyl)C(═O)aryl(C₁₋₄)alkyl, —NHC(═O)(C₁₋₄)alkoxy, —N(C₁₋₆alkyl)C(═O)(C₁₋₄)alkoxy, —NHC(═O)NH₂, —N(C₁₋₄alkyl)C(═O)NH₂, —NHC(═O)NH(C₁₋₄)alkyl, —NHC(═O)N(C₁₋₄alkyl)₂, —NHSO₂aryl, —C(═O)NH₂, —C(═O)NH(C₁₋₆alkyl), —C(═O)N(C₁₋₆alkyl)₂, and halogen;
wherein the aryl and heteroaryl substituents of Z are optionally substituted with one to four substituents independently selected from the group consisting of C₁₋₄alkyl, hydroxyC₁₋₄alkyl, C₁₋₄alkoxy, hydroxy, halogen, nitro, carboxy, amino, alkylamino, dialkylamino, —SO₂(C₁₋₄)alkyl, and —C(═O)aryl; additionally, the heteroaryl is optionally substituted with oxo;
wherein the cycloalkyl and heterocyclyl substituents of Z are optionally substituted with one to four substituents independently selected from the group consisting of C₁₋₅alkyl, C₁₋₅alkylamino, di(C₁₋₅alkyl)amino, —NH(cycloalkyl) wherein said cycloalkyl is optionally spirofused to a heterocyclyl, aminocarbonyl, —NHC(═O)C₁₋₄alkoxy, —N(C₁₋₆alkyl)C(═O)C₁₋₄alkoxy, —C(═O)(C₁₋₄)alkoxy, —NHC(═O)C₁₋₄alkyl, —N(C₁₋₆alkyl)C(═O)C₁₋₄alkyl, —C(═O)aryl(C₁₋₄)alkoxy, oxo, alkoxy, hydroxy, aryl(C₁₋₄)alkoxy, heteroaryl(C₁₋₄)alkoxy, heterocyclyl, heteroaryl optionally substituted with one to three C₁₋₂alkyl substituents, and aryl; wherein the aryl substituent is optionally substituted with one to four substituents independently selected from the group consisting of C₁₋₄alkyl, halogen, amino, alkylamino, dialkylamino, aryl, and heteroaryl;
wherein R^{d} is a substituent independently selected from the group consisting of (C₁₋₆)alkyl, —C(═O)(C₁₋₆)alkyl, —C(═O)(C₁₋₆)alkoxy, —S(═O)C₁₋₄alkyl, —SO₂C₁₋₄alkyl, —S(═O)aryl, and —SO₂aryl; wherein the alkyl and alkoxy portion of (C₁₋₆)alkyl, —C(═O)(C₁₋₆)alkyl, —C(═O)(C₁₋₆)alkoxy, —S(═O)C₁₋₄alkyl, and —SO₂C₁₋₄alkyl, are optionally substituted with one to three substitutents independently selected from the group consisting of C₁₋₃alkoxy, hydroxy, aryl, heteroaryl, and heterocyclyl; and wherein said aryl and heteroaryl are optionally substituted with one to five substituents independently selected from the group consisting of C₁₋₆alkyl, hydroxy(C₁₋₆)alkyl, C₁₋₆alkoxy, carboxy, hydroxy, cyano, nitro, amino, alkylamino, dialkylamino, —SO₂(C₁₋₃)alkyl, —SO₂aryl, —SO₂heteroaryl, trifluoromethyl, trifluoromethoxy, and halogen;
and an optical isomer, enantiomer, diastereomer, racemate, or pharmaceutically acceptable salt thereof.

2. The compound of claim 1 wherein R¹ is a substituent independently selected from the group consisting of hydrogen, C₁₋₆alkyl, C₁₋₆alkoxy, aryl, heteroaryl, heterocyclyl, benzo fused cycloalkyl, benzo fused heterocyclyl, heteroaryl fused heterocyclyl, heteroaryl fused cycloalkyl, aryloxy, heteroaryloxy, heterocyclyloxy, cycloalkyloxy, —NR¹⁰R²⁰, halogen, hydroxy, and —S(C₁₋₆)alkyl;
wherein the alkoxy substituent of R¹ is optionally substituted with one to four substituents independently selected from R^{a};
wherein R^{a} is independently selected from the group consisting of aryl, heteroaryl, heterocyclyl, cycloalkyl, carboxy, amino, alkylamino, dialkylamino, hydroxy(C₁₋₆)alkoxy, one to three halogen atoms, and hydroxy;
wherein R¹⁰ and R²⁰ are independently selected from the group consisting of hydrogen, C₁₋₆alkyl, allyl, halogenated C₁₋₆alkyl, and cycloalkyl; additionally, R¹⁰ and R²⁰ are optionally taken together with the atoms to which they are attached to form a five to seven membered monocyclic ring;
wherein the aryl and aryloxy substituents of R¹ are optionally substituted with a substituent independently selected from the group consisting of C₁₋₆alkyl, C₁₋₆alkoxy, aryl, heteroaryl, C₁₋₆alkylcarbonyl, aminocarbonyl, alkylaminocarbonyl, dialkylaminocarbonyl, hydroxy, cyano, nitro, —SO₂(C₁₋₃)alkyl, —SO₂aryl, trifluoromethyl, trifluoromethoxy, and halogen;
and wherein the heteroaryl and heterocyclyl substituents of R¹ are optionally substituted with a substituent independently selected from the group consisting of one to three C₁₋₆alkyl substituents, C₁₋₆alkoxy, aryl, heteroaryl, one to three halogen atoms, hydroxy C₁₋₆alkyl, and hydroxy;
additionally, R¹ and R² are optionally taken together with the atoms to which they are attached to form a five to seven membered carbocyclic or heterocyclic ring.

3. The compound of claim 1 wherein R¹ is selected from the group consisting of C₁₋₄alkyl, C₁₋₄alkoxy, aryl, heteroaryl, heterocyclyl, benzo fused heterocyclyl, aryloxy, heteroaryloxy, heterocyclyloxy, cycloalkyloxy, —NR¹⁰R²⁰, halogen, hydroxy, and —S(C₁₋₆)alkyl;
wherein the alkoxy substutuent of R¹ is optionally substituted with one to three substituents independently selected from R^{a};
wherein R^{a} is independently selected from the group consisting of heteroaryl, heterocyclyl, cycloalkyl, aryl, dialkylamino, hydroxy(C₁₋₆)alkoxy, one to three halogen atoms, and hydroxy;
wherein R¹⁰ and R²⁰ are independently selected from the group consisting of hydrogen, C₁₋₆alkyl, allyl, and cycloalkyl;
wherein the aryl and aryloxy substituents of R¹ are optionally substituted with a substituent independently selected from the group consisting of C₁₋₆alkyl, C₁₋₆alkoxy, phenyl, heteroaryl, aminocarbonyl, alkylaminocarbonyl, dialkylaminocarbonyl, hydroxy, cyano, nitro, —SO₂(C₁₋₃)alkyl, —SO₂aryl, trifluoromethyl, trifluoromethoxy, and halogen;
and wherein the heteroaryl and heterocyclyl substituents of R¹ are optionally substituted with a substituent independently selected from the group consisting of one to three C₁₋₆alkyl groups, halogen, and hydroxy;
additionally, R¹ and R² are optionally taken together with the atoms to which they are attached to form a five to seven membered carbocyclic or heterocyclic ring.

4. The compound of claim 1 wherein R¹ is selected from the group consisting of ethyl, methoxy, ethoxy, 2-hydroxyeth-1-oxy, iso-propoxy, iso-butoxy, difluoromethoxy, 2,2,2-trifluoro-eth-1-oxy, benzyloxy, cyclopropylmethoxy, pyridin-3-ylmethoxy, (1-methyl)-pyrrolidinyl-3-oxy, cyclobutyloxy, cyclopentyloxy, cyclohexyloxy, indazol-1-yl, thiophen-3-yl, [1,3]benzodioxol-5-yl, (2-methyl)-imidazol-1-yl, (1-methyl)-piperidin-4-yloxy, 2-(morpholin-4-yl)-ethoxy, (4-bromo)-pyrazol-1-yl, N-pyrrolidinyl, (3,5-dimethyl)-pyrazol-1-yl, morpholin-4-yl, hydroxy, —(OCH₂CH₂)₂OH, phenyl (optionally substituted with a substituent independently selected from the group consisting of —SO₂Me, —C(═O)NH₂, —OCF₃, —CF₃, cyano, fluoro, and methoxy), amino, cyclopropylamino, allylamino, methylamino, hydroxy, chloro, and —SMe;
additionally, R¹ is optionally taken together with R² to form a 1,4-dioxanyl or a oxazinyl ring.

5. The compound of claim 1 wherein R¹ is selected from the group consisting of methoxy, ethoxy, 2-hydroxyeth-1-oxy, iso-propoxy, iso-butoxy, difluoromethoxy, 2,2,2-trifluoro-eth-1-oxy, benzyloxy, cyclopropylmethoxy, pyridin-3-ylmethoxy, (1-methyl)-pyrrolidinyl-3-oxy, cyclobutyloxy, cyclopentyloxy, cyclohexyloxy, indazol-1-yl, thiophen-3-yl, [1,3]benzodioxol-5-yl, (2-methyl)-imidazol-1-yl, (1-methyl)-piperidin-4-yloxy, 2-(morpholin-4-yl)-ethoxy, (4-bromo)-pyrazol-1-yl, N-pyrrolidinyl, (3,5-dimethyl)-pyrazol-1-yl, morpholin-4-yl, hydroxy, —(OCH₂CH₂)₂OH, phenyl (optionally substituted with —SO₂Me, —C(═O)NH₂, —OCF₃, —CF₃, cyano, fluoro, or methoxy), cyclopropylamino, allylamino, and methylamino;
and wherein R¹ is optionally taken together with R² to form a 1,4-dioxanyl or a oxazinyl ring.

6. The compound of claim 1 wherein R² is a substituent independently selected from the group consisting of hydrogen, C₁₋₄alkyl, C₁₋₄alkoxy, C₂₋₄alkenyloxy, hydroxy, amino, and halogen; wherein R¹ and R² are optionally taken together with the atoms to which they are attached to form a five to seven membered carbocyclic or heterocyclic ring.

7. The compound of claim 1 wherein R is a substituent independently selected from the group consisting of hydrogen, C₁₋₄alkyl, C₁₋₄alkoxy, hydroxy, amino, alkylamino, and halogen; wherein R² is optionally taken together with R¹ to form a 1,4-dioxanyl or an oxazinyl ring.

8. The compound of claim 1 wherein R² is a substituent independently selected from the group consisting of hydrogen, C₁₋₄alkoxy, amino, and alkylamino; wherein R² is optionally taken together with R¹ to form a 1,4-dioxanyl or an oxazinyl ring.

9. The compound of claim 1 wherein R³ is a substituent independently selected from the group consisting of hydrogen, C₁₋₆alkyl, aryl, heteroaryl, heterocyclyl, and cycloalkyl;
wherein the alkyl substituent of R³ is optionally substituted with a substituent independently selected from the group consisting of —C(═O)NH₂, aryl, heteroaryl, heterocyclyl, cycloalkyl, carboxy, one to three halogen atoms, hydroxy, and —C(═O)C₁₋₆alkyl.

10. The compound of claim 1 wherein R³ is a substituent independently selected from the group consisting of hydrogen, C₁₋₄alkyl, cycloalkyl, and aryl;
wherein C₁₋₄alkyl is optionally substituted with a substituent independently selected from the group consisting of —C(═O)C₁₋₄alkyl, —C(═O)NH₂, carboxy, heterocyclyl, phenyl, cyclopropyl, hydroxy, and one to three fluorine atoms.

11. The compound of claim 1 wherein R³ is a substituent independently selected from the group consisting of hydrogen, C₁₋₄alkyl, and phenyl;
wherein C₁₋₄alkyl is optionally substituted with a substituent selected from —C(═O)C₁₋₄alkyl, —C(═O)NH₂, carboxy, morpholinyl, cyclopropyl, hydroxy, or one to three fluorine atoms.

12. The compound of claim 1 wherein R³ is a substituent independently selected from the group consisting of hydrogen, methyl, ethyl, and phenyl;
wherein methyl and ethyl are optionally substituted with a substituent independently selected from the group consisting of —C(═O)C₁₋₄alkyl, —C(═O)NH₂, carboxy, morpholinyl, cyclopropyl, hydroxy, and one to three fluorine atoms.

13. The compound of claim 1 wherein R⁴ is independently selected from the group consisting of hydrogen, fluorine, and chlorine.

14. The compound of claim 1 wherein R⁴ is hydrogen or fluorine.

15. The compound of claim 1 wherein R⁴ is hydrogen.

16. The compound of claim 1 wherein R⁵ is hydrogen or C₁₋₃alkyl, provided that R⁵ is C₁₋₃alkyl only when taken with Y and the atoms to which R⁵ and Y are attached to form a five to seven membered heterocycle.

17. The compound of claim 1 wherein R⁵ is hydrogen or methylene, provided that R⁵ is methylene only when taken with Y and the atoms to which R⁵ and Y are attached to form a five membered heterocycle.

18. The compound of claim 1 wherein R⁵ is hydrogen.

19. The compound of claim 1 wherein Y is independently selected from the group consisting of hydroxymethyl, —C(═O)NH₂, —C(═O)NH(OH), —C(═O)NH(2-hydroxyeth-1-yl), carboxy, tetrazolyl, —C(═O)NHSO₂(C₁₋₄)alkyl, and —C(═O)C₁₋₆alkoxy;
wherein said alkoxy is optionally substituted with one to two substituents independently selected from the group consisting of hydroxy, —NR³⁰R⁴⁰, heterocyclyl, heteroaryl, halogen, and —OCH₂CH₂OCH₃; wherein R³⁰ and R⁴⁰ are independently selected from the group consisting of hydrogen and C₁₋₆alkyl.

20. The compound of claim 1 wherein Y is independently selected from the group consisting of carboxy, tetrazolyl, —C(═O)NH(2-hydroxyeth-1-yl) and —C(═O)C₁₋₄alkoxy;
wherein said alkoxy is optionally substituted with one to two substituents independently selected from the group consisting of hydroxy, —NH₂, —NH(C₁₋₄)alkyl, —N(C₁₋₄alkyl)₂, heterocyclyl, halogen, and —OCH₂CH₂OCH₃.

21. The compound of claim 1 wherein Y is independently selected from the group consisting of carboxy, 1H-tetrazol-5-yl, and —C(═O)C₁₋₄alkoxy; wherein said alkoxy is optionally substituted with a substituent independently selected from hydroxy, —NMe₂, morpholin-1-yl, chloro, or —OCH₂CH₂OCH₃.

22. The compound of claim 1 wherein Y is independently selected from the group consisting of carboxy, 1H-tetrazol-5-yl, and —C(═O)ethoxy; wherein ethoxy is optionally substituted with hydroxy, chlorine, —NMe₂, and —OCH₂CH₂OCH₃.

23. The compound of claim 1 wherein Z is independently selected from the group consisting of C₁₋₆alkyl, C₁₋₆alkenyl, C₁₋₆alkoxy, aryl, heteroaryl, cycloalkyl, heterocyclyl, polycycloalkyloxy, and aza-bridged polycycyl wherein aza-bridged polycycyl is optionally substituted with R^{d};
wherein the C₁₋₆alkyl substituent of Z is optionally substituted with one to three substituents independently selected from the group consisting of aryl, aryl(C₁₋₄)alkoxy, heteroaryl optionally substituted with one to three C₁₋₂alkyl substituents, hydroxy, —NH₂, —NH(C₁₋₆alkyl), —N(C₁₋₆alkyl)₂, —NH(cycloalkyl) wherein said cycloalkyl is optionally spirofused to a heterocyclyl, —NHC(═O)aryl(C₁₋₄)alkoxy, —N(C₁₋₆alkyl)C(═O)aryl(C₁₋₄)alkoxy, —NHC(═O)heteroaryl(C₁₋₄)alkyl, —N(C₁₋₆alkyl)C(═O)heteroaryl(C₁₋₄)alkyl, —NHC(═O)aryl(C₁₋₄)alkyl, —N(C₁₋₆alkyl)C(═O)aryl(C₁₋₄)alkyl, —NHC(═O)(C₁₋₄)alkoxy, —N(C₁₋₆alkyl)C(═O)(C₁₋₄)alkoxy, —NHC(═O)NH₂, —NHSO₂aryl, —C(═O)NH₂, —C(═O)NH(C₁₋₆alkyl), —C(═O)N(C₁₋₆alkyl)₂, and halogen;
wherein the aryl and heteroaryl substituents of Z are optionally substituted with one to four substituents independently selected from the group consisting of C₁₋₄alkyl, hydroxyC₁₋₄alkyl, C₁₋₄alkoxy, hydroxy, halogen, nitro, carboxy, amino, alkylamino, dialkylamino, —SO₂(C₁₋₄)alkyl, and —C(═O)aryl; additionally, the heteroaryl is optionally substituted with oxo;
wherein the cycloalkyl and heterocyclyl substituents of Z are optionally substituted with one to four substituents independently selected from the group consisting of C₁₋₅alkyl, amino, C₁₋₅alkylamino, di(C₁₋₅alkyl)amino, —NH(cycloalkyl) wherein said cycloalkyl is optionally spirofused to a heterocyclyl, aminocarbonyl, —NHC(═O)C₁₋₄alkoxy, —N(C₁₋₆alkyl)C(═O)C₁₋₄alkoxy, —C(═O)(C₁₋₄)alkoxy, —C(═O)(C₁₋₄)alkyl, —C(═O)aryl(C₁₋₄)alkoxy, oxo, alkoxy, hydroxy, aryl(C₁₋₄)alkoxy, and aryl; wherein said aryl is optionally substituted with one to four substituents independently selected from the group consisting of C₁₋₄alkyl, halogen, amino, alkylamino, and dialkylamino.

24. The compound of claim 1 wherein Z is independently selected from the group consisting of C₁₋₆alkyl, C₁₋₆alkenyl, C₁₋₆alkoxy, aryl, heteroaryl, cycloalkyl, heterocyclyl, and aza-bridged polycycyl wherein aza-bridged polycycyl is optionally substituted with R^{d};
wherein the C₁₋₆alkyl substituent of Z is optionally substituted with one to three substituents independently selected from the group consisting of aryl, heteroaryl optionally substituted with one to three C₁₋₂alkyl substituents, hydroxy, aryl(C₁₋₄)alkoxy, —C(═O)C₁₋₆alkyl, —NH(C₁₋₆alkyl), —N(C₁₋₆alkyl)₂, —NH(cycloalkyl) wherein said cycloalkyl is optionally spirofused to a heterocyclyl, —NHC(═O)aryl(C₁₋₄)alkoxy, —N(C₁₋₆alkyl)C(═O)aryl(C₁₋₄)alkoxy, —NHC(═O)heteroaryl(C₁₋₄)alkyl, —N(C₁₋₆alkyl)C(═O)heteroaryl(C₁₋₄)alkyl, —N(C₁₋₆alkyl)C(═O)aryl(C₁₋₄)alkyl, —NHC(═O)(C₁₋₄)alkoxy, —N(C₁₋₆alkyl)C(═O)(C₁₋₄)alkoxy, —NHC(═O)NH₂, —NHSO₂aryl, and halogen;
wherein the aryl and heteroaryl substituents of Z are optionally substituted with one to four substituents independently selected from the group consisting of C₁₋₄alkyl, halogen, nitro, and —SO₂(C₁₋₄)alkyl;
wherein the cycloalkyl and heterocyclyl substituents of Z are optionally substituted with a substituent independently selected from the group consisting of one to four C₁₋₄alkyl substituents, —C(═O)NH₂, —C(═O)NH(C₁₋₄)alkyl, amino, C₁₋₄alkylamino, —NH(cycloalkyl) wherein said cycloalkyl is optionally spirofused to a heterocyclyl, —NHC(═O)C₁₋₄alkoxy, —C(═O)(C₁₋₄)alkyl, —C(═O)aryl(C₁₋₄)alkoxy, oxo, alkoxy, hydroxy, aryl(C₁₋₄)alkoxy, and aryl; wherein said aryl is optionally substituted with one to four substituents independently selected from the group consisting of C₁₋₄alkyl and halogen.

25. The compound of claim 1 wherein Z is independently selected from the group consisting of C₁₋₄alkyl, C₁₋₄alkenyl, C₁₋₄alkoxy, aryl, heteroaryl, cycloalkyl, heterocyclyl, and aza-bridged polycycyl wherein aza-bridged polycycyl is optionally substituted with R^{d};
wherein the C₁₋₄alkyl substituent of Z is optionally substituted with one to three substituents independently selected from the group consisting of aryl, heteroaryl optionally substituted with one to two methyl substituents, —NH₂, —NH(C₁₋₆alkyl), —NH(cycloalkyl), aryl(C₁₋₄)alkoxy, —N(methyl)C(═O)aryl(C₁₋₄)alkoxy, —N(methyl)C(═O)heteroaryl(C₁₋₄)alkyl, —N(methyl)C(═O)aryl(C₁₋₄)alkyl, —NHC(═O)C₁₋₄alkoxy, —N(methyl)C(═O)C₁₋₄alkoxy, and —N HC(═O)N H₂;
wherein the aryl and heteroaryl substituents of Z are optionally substituted with one to four substituents independently selected from the group consisting of C₁₋₄alkyl, halogen, and —SO₂(C₁₋₄)alkyl; additionally, the heteroaryl is optionally substituted with oxo;
wherein the cycloalkyl and heterocyclyl substituents of Z are optionally substituted with one to four substituents independently selected from the group consisting of C₁₋₄alkyl, aminocarbonyl, amino, C₁₋₄alkylamino, di(C₁₋₄)alkylamino, —NH(cycloalkyl) wherein said cycloalkyl is optionally spirofused to a heterocyclyl, —NHC(═O)C₁₋₄alkoxy, —N(C₁₋₆alkyl)C(═O)C₁₋₄alkoxy, —C(═O)(C₁₋₄)alkoxy, aryl(C₁₋₄)alkoxy, and —C(═O)aryl(C₁₋₄)alkoxy.

26. The compound of claim 1 wherein Z is independently selected from the group consisting of C₁₋₄alkyl, C₁₋₄alkenyl, C₁₋₄alkoxy, phenyl, pyrrolyl, pyridinyl, C₃₋₆cycloalkyl, tetrahydropyranyl, and 2-aza-bicyclo[2.2.2.]-octanyl wherein 2-aza-bicyclo[2.2.2]-octanyl is optionally substituted with R^{d};
wherein the C₁₋₄alkyl is optionally substituted with one to three substituents independently selected from the group consisting of phenyl, thiophenyl, pyrrolyl optionally substituted with one to two methyl substituents, —NH₂, —NH(C₁₋₆alkyl), —NH(cycloalkyl), —N(methyl)C(═O)benzyloxy, —N(methyl)C(═O)thiophenylmethyl, —N(methyl)C(═O)phenylethyl, —NHC(═O)ₜ-butoxy, —N(methyl)C(═O)ₜ-butoxy, and —NHC(═O)NH₂;
wherein phenyl and the heteroaryl substituents of Z are optionally substituted with one to four substituents independently selected from the group consisting of methyl, fluorine, chlorine, and —SO₂methyl; additionally, the heteroaryl is optionally substituted with oxo;
wherein the C₃₋₆cycloalkyl substituent of Z is optionally substituted with a substituent independently selected from the group consisting of one to four methyl substituents, —C(═O)NH₂, —C(═O)NH(i-propyl), —NHcycloalkyl wherein said cycloalkyl is optionally spirofused to a heterocyclyl, i-propyl-amino, amino, and phenyl(C₁₋₄)alkoxy; additionally, the tetrahydropyranyl substituent of Z is optionally spiro-fused to a heterocyclyl.

27. The compound of claim 1 wherein Z is independently selected from the group consisting of 2,6-dichloro-phenyl, 2-chloro-4-methanesulfonyl-phenyl, 2-chloro-5-fluoro-phenyl, 2,6-dichloro-pyridinyl-N-oxide, 3,5-dichloro-pyridin-4-yl, 1-phenyl-2-methyl-prop-1-yl, —CH(l-propyl)-N(Me)C(═O)CH₂thiophenyl, —CH(i-propyl)-NHcyclohexyl, —CH(i-propyl)-(2,5-dimethyl)-pyrrol-1-yl, —CH(l-propyl)-N(Me)ₜ-butoxy, —CH(i-propyl)-NH-t-butoxy, —CH(i-propyl)-NH(Me), (1-aminocarbonyl)-cycloprop-1-yl, (1-1-propylamino)cycloprop-1-yl, and 2-methyl-prop-2-en-1-yl.

28. The compound of claim 1 wherein R^{d} is a substituent independently selected from the group consisting of (C₁₋₆)alkyl, —C(═O)(C₁₋₆)alkyl, —C(═O)(C₁₋₆)alkoxy, —S(═O)C₁₋₄alkyl, —SO₂C₁₋₄alkyl, —S(═O)aryl, and —SO₂aryl;
wherein the alkyl and alkoxy portion of (C₁₋₆)alkyl, —C(═O)(C₁₋₆)alkyl, —C(═O)(C₁₋₆)alkoxy, —S(═O)C₁₋₄alkyl, and —SO₂C₁₋₄alkyl are optionally substituted with one to three substitutents independently selected from the group consisting of C₁₋₃alkoxy, hydroxy, aryl, heterocyclyl, and heteroaryl; wherein said aryl and heteroaryl are optionally substituted with one to five substituents independently selected from the group consisting of C₁₋₆alkyl, hydroxy(C₁₋₆)alkyl, C₁₋₆alkoxy, carboxy, hydroxy, cyano, nitro, —SO₂(C₁₋₃)alkyl, —SO₂aryl, —SO₂heteroaryl, trifluoromethyl, trifluoromethoxy, and halogen.

29. The compound of claim 1 wherein R^{d} is a substituent independently selected from the group consisting of —C(═O)(C₁₋₆)alkyl, —C(═O)(C₁₋₆)alkoxy, —S(═O)C₁₋₄alkyl, —SO₂C₁₋₄alkyl, —S(═O)aryl, and —SO₂aryl;
wherein the alkyl and alkoxy portion of (C₁₋₆)alkyl, —C(═O)(C₁₋₆)alkyl, —C(═O)(C₁₋₆)alkoxy, —S(═O)C₁₋₄alkyl, and —SO₂C₁₋₄alkyl is optionally substituted with one to three substitutents independently selected from the group consisting of C₁₋₃alkoxy, aryl, and heteroaryl.

30. The compound of claim 1 wherein R^{d} is a substituent independently selected from the group consisting of —C(═O)(C₁₋₆)alkyl, —C(═O)(C₁₋₆)alkoxy, —SO₂C₁₋₄alkyl, and —SO₂aryl;
wherein the alkyl and alkoxy portion of —C(═O)(C₁₋₆)alkyl, —C(═O)(C₁₋₆)alkoxy, and —SO₂C₁₋₄alkyl is optionally substituted with a substitutent independently selected from the group consisting of C₁₋₃alkoxy, aryl, and heteroaryl.

31. The compound of claim 1 wherein R^{d} is independently selected from the group consisting of —C(═O)(C₁₋₆)alkyl, —C(═O)(C₁₋₆)alkoxy, and —SO₂phenyl;
wherein the alkyl and alkoxy portion of —C(═O)(C₁₋₆)alkyl and —C(═O)(C₁₋₆)alkoxy is optionally substituted with a substitutent independently selected from the group consisting of methoxy, phenyl, tetrazolyl, furanyl, and thiophenyl.

32. A compound of Formula (Ia) 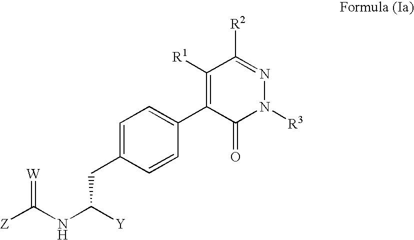
wherein R⁴ is hydrogen and R⁵ is hydrogen, and wherein R¹, R², R³, W, Y, and Z are:

| R¹ | R² | R³ | Y | W | Z |
|---|---|---|---|---|---|
| OCH₃ | H | CH₃ | CO₂H | O | (2,6-Cl₂)phenyl |

or

| | | | | | |
|---|---|---|---|---|---|
| OCH₃ | H | CH₃ | CO₂H | O | (S)—CH(i-Pr)-2,5- |
| | | | | | dimethyl- |
| | | | | | pyrrol-1-yl |

33. A compound of Formula (Ib): 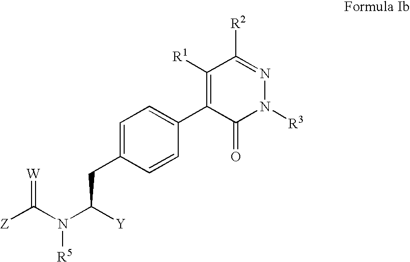
wherein R¹, R², R³, R⁵, W, Y, and Z are:

| | | | | | | |
|---|---|---|---|---|---|---|
| R¹ | R² | R³ | R⁵ | Y | W | Z |
| | | | | | |
|---|---|---|---|---|---|
| OCH₃ | H | CH₃ | —CH₂OC(═O)— | O | (2,6-Cl₂)phenyl. |

34. A compound of the Formula (Ic): 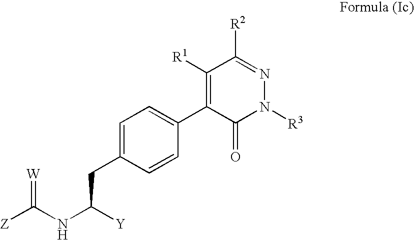
R¹ is selected from the group consisting of C₁₋₄alkyl, C₁₋₄alkoxy, aryl, heteroaryl, heterocyclyl, benzo fused heterocyclyl, aryloxy, heteroaryloxy, heterocyclyloxy, cycloalkyloxy, —NR¹⁰R²⁰, halogen, hydroxy, and —S(C₁₋₆)alkyl; wherein the alkoxy substutuent of R¹ is optionally substituted with one to three substituents independently selected from R^{a};
wherein R^{a} is independently selected from the group consisting of heteroaryl, heterocyclyl, cycloalkyl, aryl, dialkylamino, hydroxy(C₁₋₆)alkoxy, one to three halogen atoms, and hydroxy;
wherein R¹⁰ and R²⁰ are independently selected from the group consisting of hydrogen, C₁₋₆alkyl, allyl, and cycloalkyl;
wherein the aryl and aryloxy substituents of R¹ are optionally substituted with a substituent independently selected from the group consisting of C₁₋₆alkyl, C₁₋₆alkoxy, phenyl, heteroaryl, aminocarbonyl, alkylaminocarbonyl, dialkylaminocarbonyl, hydroxy, cyano, nitro, —SO₂(C₁₋₃)alkyl, —SO₂aryl, trifluoromethyl, trifluoromethoxy, and halogen;
and wherein the heteroaryl and heterocyclyl substituents of R¹ are optionally substituted with a substituent independently selected from the group consisting of one to three C₁₋₆alkyl groups, halogen, and hydroxy;
additionally, R¹ and R² are optionally taken together with the atoms to which they are attached to form a five to seven membered carbocyclic or heterocyclic ring;
R² is a substituent independently selected from the group consisting of hydrogen, C₁₋₄alkyl, C₁₋₄alkoxy, C₂₋₄alkenyloxy, hydroxy, amino, and halogen; wherein R¹ and R² are optionally taken together with the atoms to which they are attached to form a five to seven membered carbocyclic or heterocyclic ring;
R³ is a substituent independently selected from the group consisting of hydrogen, C₁₋₄alkyl, cycloalkyl, and aryl; wherein C₁₋₄alkyl is optionally substituted with a substituent independently selected from the group consisting of —C(═O)C₁₋₄alkyl, —C(═O)NH₂, carboxy, heterocyclyl, phenyl, cyclopropyl, hydroxy, and one to three fluorine atoms;
Y is independently selected from the group consisting of carboxy, tetrazolyl, —C(═O)NH(2-hydroxyeth-1-yl) and —C(═O)C₁₋₄alkoxy; wherein said alkoxy is optionally substituted with one to two substituents independently selected from the group consisting of hydroxy, —NH₂, —NH(C₁₋₄)alkyl, —N(C₁₋₄alkyl)₂, heterocyclyl, halogen, and —OCH₂CH₂OCH₃;
Z is independently selected from the group consisting of C₁₋₆alkyl, C₁₋₆alkenyl, C₁₋₆alkoxy, aryl, heteroaryl, cycloalkyl, heterocyclyl, and aza-bridged polycycyl wherein aza-bridged polycycyl is optionally substituted with R^{d};
wherein the C₁₋₆alkyl substituent of Z is optionally substituted with one to three substituents independently selected from the group consisting of aryl, heteroaryl optionally substituted with one to three C₁₋₂alkyl substituents, hydroxy, aryl(C₁₋₄)alkoxy, —C(═O)C₁₋₆alkyl, —NH(C₁₋₆alkyl), —N(C₁₋₆alkyl)₂, —NH(cycloalkyl) wherein said cycloalkyl is optionally spirofused to a heterocyclyl, —NHC(═O)aryl(C₁₋₄)alkoxy, —N(C₁₋₆alkyl)C(═O)aryl(C₁₋₄)alkoxy, —NHC(═O)heteroaryl(C₁₋₄)alkyl, —N(C₁₋₆alkyl)C(═O)heteroaryl(C₁₋₄)alkyl, —N(C₁₋₆alkyl)C(═O)aryl(C₁₋₄)alkyl, —NHC(═O)(C₁₋₄)alkoxy, —N(C₁₋₆alkyl)C(═O)(C₁₋₄)alkoxy, —NHC(═O)NH₂, —NHSO₂aryl, and halogen;
wherein the aryl and heteroaryl substituents of Z are optionally substituted with one to four substituents independently selected from the group consisting of C₁₋₄alkyl, halogen, nitro, and —SO₂(C₁₋₄)alkyl;
wherein the cycloalkyl and heterocyclyl substituents of Z are optionally substituted with a substituent independently selected from the group consisting of one to four C₁₋₄alkyl substituents, —C(═O)NH₂, —C(═O)NH(C₁₋₄)alkyl, amino, C₁₋₄alkylamino, —NH(cycloalkyl) wherein said cycloalkyl is optionally spirofused to a heterocyclyl, —NHC(═O)C₁₋₄alkoxy, —C(═O)(C₁₋₄)alkyl, —C(═O)aryl(C₁₋₄)alkoxy, oxo, alkoxy, hydroxy, aryl(C₁₋₄)alkoxy, and aryl; wherein said aryl is optionally substituted with one to four substituents independently selected from the group consisting of C₁₋₄alkyl and halogen;
R^{d} is a substituent independently selected from the group consisting of —C(═O)(C₁₋₆)alkyl, —C(═O)(C₁₋₆)alkoxy, —S(═O)C₁₋₄alkyl, —SO₂C₁₋₄alkyl, —S(═O)aryl, and —SO₂aryl;
wherein the alkyl and alkoxy portion of (C₁₋₆)alkyl, —C(═O)(C₁₋₆)alkyl, —C(═O)(C₁₋₆)alkoxy, —S(═O)C₁₋₄alkyl, and —SO₂C₁₋₄alkyl is optionally substituted with one to three substitutents independently selected from the group consisting of C₁₋₃alkoxy, aryl, and heteroaryl
and an optical isomer, enantiomer, diastereomer, racemate or pharmaceutically acceptable salts thereof.

35. The compound of claim 34 wherein R¹ is selected from the group consisting of ethyl, methoxy, ethoxy, 2-hydroxyeth-1-oxy, iso-propoxy, iso-butoxy, difluoromethoxy, 2,2,2-trifluoro-eth-1-oxy, benzyloxy, cyclopropylmethoxy, pyridin-3-ylmethoxy, (1-methyl)-pyrrolidinyl-3-oxy, cyclobutyloxy, cyclopentyloxy, cyclohexyloxy, indazol-1-yl, thiophen-3-yl, [1,3]benzodioxol-5-yl, (2-methyl)-imidazol-1-yl, (1-methyl)-piperidin-4-yloxy, 2-(morpholin-4-yl)-ethoxy, (4-bromo)-pyrazol-1-yl, N-pyrrolidinyl, (3,5-dimethyl)-pyrazol-1-yl, morpholin-4-yl, hydroxy, —(OCH₂CH₂)₂OH, phenyl (optionally substituted with a substituent independently selected from the group consisting of —SO₂Me, —C(═O)NH₂, —OCF₃, —CF₃, cyano, fluoro, and methoxy), amino, cyclopropylamino, allylamino, methylamino, hydroxy, chloro, and —SMe;
additionally, R¹ is optionally taken together with R² to form a 1,4-dioxanyl or a oxazinyl ring.

36. The compound of claim 34 wherein R² is a substituent independently selected from the group consisting of hydrogen, C₁₋₄alkyl, C₁₋₄alkoxy, hydroxy, amino, alkylamino, and halogen; wherein R² is optionally taken together with R¹ to form a 1,4-dioxanyl or an oxazinyl ring.

37. The compound of claim 34 wherein R³ is a substituent independently selected from the group consisting of hydrogen, C₁₋₄alkyl, and phenyl;
wherein C₁₋₄alkyl is optionally substituted with a substituent selected from —C(═O)C₁₋₄alkyl, —C(═O)NH₂, carboxy, morpholinyl, cyclopropyl, hydroxy, or one to three fluorine atoms.

38. The compound of claim 34 wherein Y is independently selected from the group consisting of carboxy, 1H-tetrazol-5-yl, and —C(═O)C₁₋₄alkoxy; wherein said alkoxy is optionally substituted with a substituent independently selected from hydroxy, —NMe₂, morpholin-1-yl, chloro, or —OCH₂CH₂OCH₃.

39. The compound of claim 34 wherein Z is independently selected from the group consisting of C₁₋₄alkyl, C₁₋₄alkenyl, C₁₋₄alkoxy, aryl, heteroaryl, cycloalkyl, heterocyclyl, and aza-bridged polycycyl wherein aza-bridged polycycyl is optionally substituted with R^{d};
wherein the C₁₋₄alkyl substituent of Z is optionally substituted with one to three substituents independently selected from the group consisting of aryl, heteroaryl optionally substituted with one to two methyl substituents, —NH₂, —NH(C₁₋₆alkyl), —NH(cycloalkyl), aryl(C₁₋₄)alkoxy, —N(methyl)C(═O)aryl(C₁₋₄)alkoxy, —N(methyl)C(═O)heteroaryl(C₁₋₄)alkyl, —N(methyl)C(═O)aryl(C₁₋₄)alkyl, —NHC(═O)C₁₋₄alkoxy, —N(methyl)C(═O)C₁₋₄alkoxy, and —N HC(═O)NH₂;
wherein the aryl and heteroaryl substituents of Z are optionally substituted with one to four substituents independently selected from the group consisting of C₁₋₄alkyl, halogen, and —SO₂(C₁₋₄)alkyl; additionally, the heteroaryl is optionally substituted with oxo;
wherein the cycloalkyl and heterocyclyl substituents of Z are optionally substituted with one to four substituents independently selected from the group consisting of C₁₋₄alkyl, aminocarbonyl, amino, C₁₋₄alkylamino, di(C₁₋₄)alkylamino, —NH(cycloalkyl) wherein said cycloalkyl is optionally spirofused to a heterocyclyl, —NHC(═O)C₁₋₄alkoxy, —N(C₁₋₆alkyl)C(═O)C₁₋₄alkoxy, —C(═O)(C₁₋₄)alkoxy, aryl(C₁₋₄)alkoxy, and —C(═O)aryl(C₁₋₄)alkoxy.

40. The compound of claim 34 wherein R^{d} is a substituent independently selected from the group consisting of —C(═O)(C₁₋₆)alkyl, —C(═O)(C₁₋₆)alkoxy, —SO₂C₁₋₄alkyl, and —SO₂aryl; wherein the alkyl and alkoxy portion of —C(═O)(C₁₋₆)alkyl, —C(═O)(C₁₋₆)alkoxy, and —SO₂C₁₋₄alkyl is optionally substituted with a substitutent independently selected from the group consisting of C₁₋₃alkoxy, aryl, and heteroaryl.

41. The compound of claim 34 wherein R¹, R², R³, W, Y, and Z are dependently selected from the group consisting of:

| | | | | | | Stereo |
|---|---|---|---|---|---|---|
| | | | | | | chem |
| R¹ | R² | R³ | Y | W | Z | of Z |
| OCH₃ | H | CH₃ | —CO₂Et | O | (2,6-Cl₂)phenyl | |
| OCH₃ | H | CH₃ | —C(═O) | O | (2-Cl, 5-F) | |
| | | | O(CH₂)₂ | | phenyl | |
| | | | OH | | | |
| OCH₃ | H | CH₃ | CO₂H | O | 1-(i-Pr-amino)- | |
| | | | | | cycloprop-1-yl | |
| OEt | H | CH₃ | CO₂H | O | (2,6-Cl₂)phenyl | |
| OCH₃ | H | —CH₂ | CO₂H | O | (2,6-Cl₂)phenyl | |
| | | C(═O)NH₂ | | | | |
| —OCH₂CH₂O— | | CH₃ | CO₂H | O | (2,6-Cl₂)phenyl | |
| —(OCH₂ | H | CH₃ | CO₂H | O | (2,6-Cl₂)phenyl | |
| CH₂)₂OH | | | | | | |
| (2-OH) | H | CH₃ | CO₂H | O | (2,6-Cl₂)phenyl | |
| eth-1-oxy | | | | | | |
| OCH₃ | H | CH₃ | CO₂H | O | (3,5- | |
| | | | | | Cl₂)pyridin-4- | |
| | | | | | yl- | |
| | | | | | N-oxide | |
| OCH₃ | H | 2- | CO₂H | O | (2,6-Cl₂)phenyl | |
| | | (morpholin- | | | | |
| | | 4-yl) | | | | |
| | | eth-1-yl | | | | |
| OCH₃ | H | —CH₂ | CO₂H | O | (2,6-Cl₂)phenyl | |
| | | CO₂H | | | | |
| (1-Me) | H | CH₃ | CO₂H | O | (2,6-Cl₂)phenyl | |
| pyrrolidin-3-yloxy | | | | | | |
| —NHCH₂CH₂O— | | CH₃ | CO₂H | O | (2,6-Cl₂)phenyl | |
| (4-SO₂Me) | H | CH₃ | CO₂H | O | (2,6-Cl₂)phenyl | |
| phenyl | | | | | | |
| OCH₃ | H | (2-OH) | CO₂H | O | (2,6-Cl₂)phenyl | |
| | | eth-1-yl | | | | |
| 4-(C(═O) | H | CH₃ | CO₂H | O | (2,6-Cl₂)phenyl | |
| NH₂)phenyl | | | | | | |
| OCH₃ | H | CH₃ | CO₂H | O | (2,6-Cl₂)phenyl | |
| NHMe | H | —CH₂ | CO₂H | O | (2,6-Cl₂)phenyl | |
| | | C(═O)Me | | | | |
| OCH₃ | H | CH₃ | CO₂H | O | —CH(i- | R |
| | | | | | Pr)N(Me)C(═O) | |
| | | | | | CH₂-thiophen- | |
| | | | | | 3-yl | |
| morpholin-4-yl) | H | CH₃ | CO₂H | O | (2,6-Cl₂)phenyl | |
| 2-(morpholin-4-yl) | H | CH₃ | CO₂H | O | (2,6-Cl₂)phenyl | |
| ethoxy | | | | | | |
| (2-morpholin-4-yl) | H | (2-OH) | CO₂H | O | (2,6-Cl₂)phenyl | |
| ethoxy | | eth-1-yl | | | | |
| (1-Me) | H | (2-OH) | CO₂H | O | (2,6-Cl₂)phenyl | |
| piperidin-4-yloxy | | eth-1-yl | | | | |
| i-Propoxy | H | (2-OH) | CO₂H | O | (2,6-Cl₂)phenyl | |
| | | eth-1-yl | | | | |
| pyridin-3-yl | H | CH₃ | CO₂H | O | (2,6-Cl₂)phenyl | |
| methoxy | | | | | | |
| 2-OH) | H | (2-OH) | CO₂H | O | (2,6-Cl₂)phenyl | |
| eth-1-yl | | eth-1-yl | | | | |
| [1,3]benzo | H | CH₃ | CO₂H | O | (2,6-Cl₂)phenyl | |
| dioxol-5-yl | | | | | | |
| OCH₃ | H | CH₃ | CO₂H | O | —CH(i-Pr) | |
| | | | | | NH(cyclohexyl) | |
| morpholin-4-yl | H | Et | CO₂H | O | (2,6-Cl₂)phenyl | |
| pyridin-3-yl | H | (2-OH) | CO₂H | O | (2,6-Cl₂)phenyl | |
| methoxy | | eth-1-yl | | | | |
| OCH₃ | H | CH₃ | CO₂H | O | (2-Cl, 4- | |
| | | | | | SO₂Me) | |
| | | | | | phenyl | |
| (2-Me) | H | CH₃ | CO₂H | O | (2,6-Cl₂)phenyl | |
| imidazol-1-yl | | | | | | |
| OCH₃ | H | CH₃ | CO₂H | O | —CH(i-Pr)(2,5- | d |
| | | | | | Me₂)-pyrrol-1- | |
| | | | | | yl | |
| OCH₃ | H | CH₃ | —C(═O) | O | (2,6-Cl₂)phenyl | |
| | | | O(CH₂)₂ | | | |
| | | | NMe₂ | | | |
| cyclo | H | (2-OH) | CO₂H | O | (2,6-Cl₂)phenyl | |
| propyl | | eth-1-yl | | | | |
| methoxy | | | | | | |
| —NH(allyl) | H | —CH₂ | CO₂H | O | (2,6-Cl₂)phenyl | |
| | | C(═O)Me | | | | |
| (2,2,2-F3) | H | CH₃ | CO₂H | O | (2,6-Cl₂)phenyl | |
| eth-1-oxy | | | | | | |
| OCH₃ | H | CH₃ | CO₂H | O | 1—(C(═O)NH₂)- | |
| | | | | | cycloprop-1-yl | |
| OCH₃ | H | (2,2,2-F₃) | CO₂H | O | (2,6-Cl₂)phenyl | |
| | | eth-1-yl | | | | |
| OCH₃ | H | CH₃ | CO₂H | O | 1- | |
| | | | | | (cyclohexylami | |
| | | | | | no)-cycloprop- | |
| | | | | | 1-yl | |
| cyclohexyloxy | H | CH₃ | CO₂H | O | (2,6-Cl₂)phenyl | |
| OCH₃ | H | CH₃ | CO₂H | O | 4-(i-Pr-amino)- | |
| | | | | | tetrahydro- | |
| | | | | | pyran-4-yl | |
| cyclopentyloxy | H | (2-OH) | CO₂H | O | (2,6-Cl₂)phenyl | |
| | | eth-1-yl | | | | |
| OCH₃ | H | CH₃ | CO₂H | | (2-Cl, 5- | |
| | | | | | F)phenyl | |
| OCH₃ | H | CH₃ | CO₂H | | 2-methyl- | |
| | | | | | prop-2-en-1-yl | |
| NH₂ | H | CH₃ | CO₂H | O | (2,6-Cl₂)phenyl | |
| (4-OMe) | H | CH₃ | CO₂H | O | (2,6-Cl₂)phenyl | |
| phenyl | | | | | | |
| OCH₃ | H | CH₃ | CO₂H | O | —CH(i-Pr)N(Me) | S |
| | | | | | (C(═O)OtBu) | |
| Cl | H | CH₃ | CO₂H | O | (2,6-Cl₂)phenyl | |
| OCH₃ | H | CH₃ | CO₂H | O | —CH(Me)N(Me) | R |
| | | | | | C(═O)CH₂— | |
| | | | | | thiophen-3-yl | |
| pyrrolidin-1-yl | H | CH₃ | CO₂H | O | (2,6-Cl₂)phenyl | |
| benzyloxy | H | CH₃ | CO₂H | O | (2,6-Cl₂)phenyl | |
| cyclobutyloxy | H | (2-OH) | CO₂H | O | (2,6-Cl₂)phenyl | |
| | | eth-1-yl | | | | |
| OCH₃ | H | CH₃ | CO₂H | O | —CH(i- | S |
| | | | | | Pr)NH(Me) | |
| OCH₃ | H | CH₃ | CO₂H | O | (3,5- | |
| | | | | | Cl₂)pyridin-4-yl | |
| OCH₃ | H | Ph | CO₂H | O | (2,6-Cl₂)phenyl | |
| OCH₃ | H | CH₃ | CO₂H | O | 1-Ph-2-methyl- | d |
| | | | | | prop-1-yl | |
| OCH₃ | H | CH₃ | CO₂H | O | —CH(i-Pr)NH | |
| | | | | | C(═O)OtBu | d |
| (4-F)phenyl | H | CH₃ | CO₂H | O | (2,6-Cl₂)phenyl | |
| cyclopentyloxy | H | CH₃ | CO₂H | O | (2,6-Cl₂)phenyl | |
| morpholin-4-yl | H | (2-OH) | CO₂H | O | (2,6-Cl₂)phenyl | |
| | | eth-1-yl | | | | |
| OH | H | CH₃ | CO₂H | O | (3,5- | |
| | | | | | Cl₂)pyridin-4-yl | |
| phenyl | H | CH₃ | CO₂H | O | (2,6-Cl₂)phenyl | |
| (3-CF₃)phenyl | H | CH₃ | CO₂H | O | (2,6-Cl₂)phenyl | |
| (4-SO₂Me) | H | cyclopropylmethyl | CO₂H | O | (2,6-Cl₂)phenyl | |
| phenyl | | | | | | |
| (4-CN)phenyl | H | CH₃ | CO₂H | O | (2,6-Cl₂)phenyl | |
| OCH₃ | H | CH₃ | CO₂H | O | 1- | 1S, |
| | | | | | (methylamino)- | 2R |
| | | | | | 2-(benzyloxy)- | |
| | | | | | prop-1-yl | |
| (3,5-Me₂) | H | CH₃ | CO₂H | O | (2,6-Cl₂)phenyl | |
| pyrazol-1-yl | | | | | | |
| OCH₃ | H | CH₃ | CO₂H | O | —CH(i-Pr)NH(4- | d |
| | | | | | (1,4- | |
| | | | | | dioxaspiro[4.5] | |
| | | | | | decan-1-yl)) | |
| (3-OCF₃) | H | CH₃ | CO₂H | O | (2,6-Cl₂)phenyl | |
| phenyl | | | | | | |
| 1-Me) | H | (2-OH) | CO₂H | O | (2,6-Cl₂)phenyl | |
| pyrrolidinyl-3-oxy | | eth-1-yl | | | | |
| i-Propoxy | H | CH₃ | CO₂H | O | (2,6-Cl₂)phenyl | |
| OCH₃ | H | CH₃ | —C(═O) | O | (2,6-Cl₂)phenyl | |
| | | | O(CH₂)₂ | | | |
| | | | Cl | | | |
| OCH₃ | H | CH₃ | CO₂H | O | (2-Cl)pyridin-3- | |
| | | | | | yl | |
| OCH₃ | H | CH₃ | CO₂H | O | —CH(i- | d |
| | | | | | Pr)NHSO₂(2- | |
| | | | | | NO₂)phenyl | |
| SCH₃ | H | t-Bu | CO₂H | O | (2,6-Cl₂)phenyl | |
| indazol-1-yl | H | CH₃ | CO₂H | O | (2,6-Cl₂)phenyl | |
| OCH₃ | H | CH₃ | CO₂H | O | 1-(2,6-Me₂- | |
| | | | | | pyrrol-1-yl)- | |
| | | | | | cycloprop-1-yl | |
| OCH₃ | H | CH₃ | CO₂H | O | —CH(i-Pr)NH | R |
| | | | | | (C(═O)OtBu | |
| (4-Br) | H | CH₃ | CO₂H | O | (2,6-Cl₂)phenyl | |
| pyrazol-1-yl | | | | | | |
| OCH₃ | H | H | CO₂H | O | (2,6-Cl₂)phenyl | |
| OCH₃ | H | CH₃ | CO₂H | O | pyrrol-2-yl | |
| OCH₃ | H | t-Bu | CO₂H | O | (2,6-Cl₂)phenyl | |
| cyclopropyl | H | CH₃ | CO₂H | O | (2,6-Cl₂)phenyl | |
| methoxy | | | | | | |
| OCH₃ | H | CH₃ | CO₂H | O | —CH(i- | R |
| | | | | | Pr)pyrrol-1-yl | |
| indazol-1-yl | H | (2-OH) | CO₂H | O | (2,6-Cl₂)phenyl | |
| | | eth-1-yl | | | | |
| OCH₃ | H | CH₃ | CO₂H | O | 2-(phenylethyl | |
| | | | | | carbonyl)-2- | |
| | | | | | aza-bicyclo | |
| | | | | | [2.2.2]-octan- | |
| | | | | | 1-yl | |
| OCH₃ | H | CH₃ | CO₂H | O | —CH(i-Pr)N(Me) | R |
| | | | | | C(═O)(CH₂)₂Ph | |
| OCH₃ | H | CH₃ | —CO₂ | O | (2,6-Cl₂)phenyl | |
| | | | (CH₂CH₂ | | | |
| | | | O)₂Me | | | |
| —NH | H | —CH₂ | CO₂H | O | (2,6-Cl₂)phenyl | |
| (cyclopropyl) | | C(═O)t-Bu | | | | |
| Cl | H | cyclopropyl | CO₂H | O | (2,6-Cl₂)phenyl | |
| | | methyl | | | | |
| (4-Br) | H | (2-OH) | CO₂H | O | (2,6-Cl₂)phenyl | |
| pyrazol-1-yl | | eth-1-yl | | | | |
| OH | H | CH₃ | CO₂H | O | (2,6-Cl₂)phenyl | |
| OCH₃ | H | CH₃ | CO₂H | O | —CH₂N(Me) | |
| | | | | | C(═O)OBn | |
| OCH₃ | H | CH₃ | —C(═O) | O | (2,6-Cl₂)phenyl | |
| | | | O(CH₂)₂ | | | |
| | | | OH | | | |
| OCH₃ | H | CH₃ | CO₂H | O | 4-(cycloHexylamino)- | |
| | | | | | tetrahydropyran- | |
| | | | | | 4-yl | |
| OCH₃ | H | CH₃ | CO₂H | O | 2-aza- | S |
| | | | | | bicyclo[2.2.2]- | |
| | | | | | octan-1-yl | |
| OCH₃ | H | CH₃ | CO₂H | O | 2-(3-methyl- | S |
| | | | | | but-1-yl | |
| | | | | | carbonyl)-2- | |
| | | | | | aza- | |
| | | | | | bicyclo[2.2.2]- | |
| | | | | | octan-1-yl | |
| Et | H | CH₃ | CO₂H | O | (2,6-Cl₂)phenyl | |
| OCH₃ | H | CH₃ | CO₂H | O | 2-(1H- | S |
| | | | | | tetrazolylmethylcarbonyl)- | |
| | | | | | 2- | |
| | | | | | aza-bicyclo | |
| | | | | | [2.2.2]- | |
| | | | | | octan-1-yl | |
| OCH₃ | H | CH₃ | CO₂H | O | 2- | S |
| | | | | | (phenylmethoxycarbonyl)- | |
| | | | | | 2-aza- | |
| | | | | | bicyclo[2.2.2]- | |
| | | | | | octan-1-yl | |
| thiophen-3-yl | H | CH₃ | CO₂H | O | (2,6-Cl₂)phenyl | |
| OCH₃ | H | CH₃ | 1H- | O | (2,6-Cl₂)phenyl | |
| | | | tetrazol- | | | |
| | | | 5-yl | | | |
| OCH₃ | H | CH₃ | CO₂H | O | —CH(i- | R |
| | | | | | Pr)NHC(═O) | |
| | | | | | CH₂thiophen- | |
| | | | | | 3-yl | |
| OCH₃ | H | CH₃ | CO₂H | O | 2- | S |
| | | | | | (phenylsulfonyl)- | |
| | | | | | 2-aza-bicyclo | |
| | | | | | [2.2.2]-octan- | |
| | | | | | 1-yl | |
| CH₃ | H | CH₃ | CO₂H | O | 2,6-Cl₂)phenyl | |
| OCH₃ | H | CH₃ | CO₂H | O | (1-Me)-pyrrol- | |
| | | | | | 2-yl | |
| —O(i-Bu) | H | CH₃ | CO₂H | O | —O(i-Bu) | |
| OCHF₂ | H | CH₃ | CO₂H | O | (2,6-Cl₂)phenyl | |
| OCH₃ | H | CH₃ | CO₂H | O | 2-(thiophen3- | S |
| | | | | | ylmethylcarbonyl)- | |
| | | | | | 2-aza- | |
| | | | | | bicyclo[2.2.2]- | |
| | | | | | octan-1-yl | |
| OCH₃ | H | CH₃ | CO₂H | O | 2-(furan-2- | S |
| | | | | | ylethylcarbonyl)- | |
| | | | | | 2-aza- | |
| | | | | | bicyclo[2.2.2]- | |
| | | | | | octan-1-yl | |
| OCH₃ | H | CH₃ | CO₂H | O | 4-NH₂- | |
| | | | | | tetrahydropyran- | |
| | | | | | 4-yl | |
| OCH₃ | H | Bn | CO₂H | O | (2,6-Cl₂)phenyl | |
| OCH₃ | H | CH₃ | —C(═O) | O | (2,6-Cl₂)phenyl | |
| | | | O(CH₂)₂ | | | |
| | | | morpholin- | | | |
| | | | 1-yl | | | |
| OCH₃ | H | CH₃ | —C(═O) | O | (2,6-Cl₂)phenyl | |
| | | | NH(CH₂)₂OH | | | |
| OCH₃ | H | (2—OH) | CO₂H | O | —O(t-Bu) | |
| | | eth-1-yl | | | | |
| —CH₂ | H | CH₃ | CO₂H | O | (2,6-Cl₂)phenyl | |
| C(═O)OEt | | | | | | |
| OCH₃ | H | CH₃ | CO₂H | O | 2-(1H- | S |
| | | | | | imidazol-4- | |
| | | | | | ylethyl | |
| | | | | | carbonyl)-2- | |
| | | | | | aza-bicyclo | |
| | | | | | [2.2.2]-octan- | |
| | | | | | 1-yl | |
| OCH₃ | H | CH₃ | CO₂H | O | —(i- | R |
| | | | | | Pr)CH(NHMe)- | |
| OCH₃ | H | CH₃ | CO₂H | O | 2-methyl-prop- | |
| | | | | | 1-yl | |
| OCH₃ | H | CH₃ | CO₂H | O | 2-(2-methoxy- | S |
| | | | | | eth-1- | |
| | | | | | ylcarbonyl)-2- | |
| | | | | | aza-bicyclo | |
| | | | | | [2.2.2]-octan- | |
| | | | | | 1-yl | |
| OCH₃ | H | CH₃ | CO₂H | O | 2-(2-t- | S |
| | | | | | butoxycarbonyl)- | |
| | | | | | 2-aza- | |
| | | | | | bicyclo[2.2.2]- | |
| | | | | | octan-1-yl | |
| OCH₃ | H | CH₃ | CO₂H | O | 2-methyl-1- | S |
| | | | | | hydroxy-prop- | |
| | | | | | 1-yl | |
| OCH₃ | H | 2- | CO₂H | O | 1- | 1S, |
| | | (morpholin- | | | methylamino- | 2R |
| | | 4-yl)- | | | 2-benzyloxy- | |
| | | eth-1-yl | | | prop-1-yl | |
| OCH₃ | H | CH₃ | CO₂H | O | 2-methyl-1- | R |
| | | | | | hydroxy-prop- | |
| | | | | | 1-yl | |
| OCH₃ | H | CH₃ | CO₂H | O | (7- | |
| | | | | | OMe)chromen- | |
| | | | | | 2-one-3-yl | |
| 5-(thiophen-2-yl) | H | (2-OH) | CO₂H | O | (2,6-Cl₂)phenyl | |
| pyrazol-1-yl | | eth-1-yl | | | | |
| OCH₃ | H | CH₃ | CO₂H | O | 1-(4-F-phenyl)- | |
| | | | | | cyclopent-1-yl | |
| OCH₃ | H | CH₃ | CO₂H | O | 1-{{4-[1-Me, 4- | |
| | | | | | OMe- | |
| | | | | | pyridazin-5- | |
| | | | | | one]-phenyl}- | |
| | | | | | 1-carboxy-eth- | |
| | | | | | 1-ylamino | |
| | | | | | carbonyl}- | |
| | | | | | cycloprop-1-yl | |
| OCH₃ | H | CH₃ | CO₂H | O | 2-(methyl)-2- | S |
| | | | | | aza- | |
| | | | | | bicyclo[2.2.2]- | |
| | | | | | octan-1-yl | |
| OCH₃ | H | CH₃ | CO₂H | O | (2,2,3,3-Me₄) | |
| | | | | | cycloprop-1-yl | |
| OCH₃ | H | CH₃ | CO₂H | O | (4- | |
| | | | | | CO₂H)phenyl | |
| OCH₃ | H | CH₃ | CO₂H | O | (2-NH₂, 4,6- | |
| | | | | | Me₂) | |
| | | | | | pyridin-3-yl | |
| OCH₃ | H | CH₃ | CO₂H | O | 2-(2-(piperidin- | S |
| | | | | | 4-yl)-eth-1- | |
| | | | | | ylcarbonyl)-2- | |
| | | | | | aza- | |
| | | | | | bicyclo[2.2.2]- | |
| | | | | | octan-1-yl | |
| OCH₃ | H | CH₃ | CO₂H | S | (2,6-Cl₂)phenyl | |
| OCH₃ | H | CH₃ | CH₂OH | O | (2,6-Cl₂)phenyl | |
| OEt | H | t-Bu | CO₂H | O | (2,6-Cl₂)phenyl | |
| OCH₃ | H | CH₃ | CO₂H | O | —C(═O)i-Pr | |
| OCH₃ | H | CH₃ | CO₂H | O | (3,5-Me₂) | |
| | | | | | isoxazol-4-yl | |
| OCH₃ | H | CH₃ | CO₂H | O | thiophen-3- | |
| | | | | | ylmethyl | |
| OCH₃ | H | CH₃ | CO₂H | O | 1-(i- | |
| | | | | | Propylamino)- | |
| | | | | | cycloprop-1-yl | |
| OCH₃ | H | CH₃ | CO₂H | O | (5-Me) | |
| | | | | | isoxazol-4-yl | |
| 5-(thiophen-2-yl) | H | CH₃ | CO₂H | O | (2,6-Cl₂)phenyl | |
| pyrazol-1-yl | | | | | | |
| (2-NMe₂) | H | CH₃ | CO₂H | O | (2,6-Cl₂)phenyl | |
| ethoxy | | | | | | |
| OCH₃ | H | CH₃ | —CO₂Me | O | (2,6-Cl₂)phenyl | |
| CH₂CO₂H | H | CH₃ | CO₂H | O | (2,6-Cl₂)phenyl | |
| OCH₃ | H | CH₃ | CO₂H | O | —CH(i-Pr)NH(i- | R |
| | | | | | Pr) | |
| OCH₃ | H | 2- | CO₂H | O | —CH(1-OH-eth- | 1S, |
| | | (morpholin- | | | 1-yl)NH | 2R |
| | | 4-yl)eth-1- | | | C(═O)Ot-Bu | |
| | | yl | | | | |
| NMe₂ | H | t-Bu | CO₂H | O | (2,6-Cl₂)phenyl | |
| NHMe | H | CH₃ | CO₂H | O | (2,6-Cl₂)phenyl | |
| OCH₃ | H | CH₃ | CO₂H | O | —CH₂N(Me) | |
| | | | | | C(═O)Ot-Bu | |
| H | H | CH₃ | CO₂H | O | (2,6-Cl₂)phenyl | |
| OCH₃ | H | CH₃ | CO₂H | O | —CH(i-Pr)NH₂ | |
| [1,2,4 | H | t-Bu | CO₂H | O | (2,6-Cl₂)phenyl | |
| triazol-1-yl | | | | | | |
| OCH₃ | H | CH₃ | CO₂H | O | 2-(C(═O)OBn) | |
| | | | | | pyrrolidin-2-yl | |
| (2-Cl) | H | CH₃ | CO₂H | O | (2,6-Cl₂)phenyl | |
| ethylamino | | | | | | |
| OCH₃ | H | CH₃ | CO₂H | O | 1H-pyrimadin- | |
| | | | | | 2,4-dione-6-yl | |
| OCH₃ | H | CH₃ | CO₂H | O | 1- | |
| | | | | | (benzyloxycarbonyl) | |
| | | | | | piperidin-4-yl | |
| OCH₃ | H | cyclohexyl | CO₂H | O | (2,6-Cl₂)phenyl | |
| OCH₃ | H | CH₃ | CO₂H | O | pyrrolidin-2-yl | d |
| OCH₃ | H | 2- | CO₂H | O | 2-hydroxy-1-(t- | 1R, |
| | | (morpholi | | | butoxycarbonylamino)- | 2S |
| | | n-4-yl) | | | prop-1- | |
| | | eth-1-yl | | | yl | |
| OH | H | H | CO₂H | | (2,6-Cl₂)phenyl | |
| OCH₃ | H | CH₃ | CO₂H | O | (2,6- | |
| | | | | | Cl₂)Pyridin-2-yl | |
| OCH₃ | H | CH₃ | CO₂H | O | (4- | |
| | | | | | hydroxymethyl) | |
| | | | | | phenyl | |
| OCH₃ | H | CH₃ | CO₂H | O | neopentyloxy | |
| OCH₃ | H | CH₃ | CO₂H | O | benzyloxy | |
| OCH₃ | H | CH₃ | CO₂H | O | —CH₂NMe₂ | |
| OCH₃ | H | CH₃ | CO₂H | O | 2-(3-hydroxy- | S |
| | | | | | 3-methyl-prop- | |
| | | | | | 1-ylcarbonyl)- | |
| | | | | | 2-aza-bicyclo | |
| | | | | | [2.2.2]- | |
| | | | | | octan-1-yl | |
| OCH₃ | H | CH₃ | CO₂H | O | —O(i-Bu) | |
| OCH₃ | H | CH₃ | —C(═O) | O | (2,6-Cl₂)phenyl | |
| | | | NH(OH) | | | |
| OCH₃ | H | CH₃ | CO₂H | O | 1-(t-Butoxycarbonylamino)- | |
| | | | | | cycloprop-1- | |
| | | | | | yl | |
| OCH₃ | H | CH₃ | CO₂H | O | OMe | |
| OCH₃ | H | 2- | CO₂H | O | —CH(i-Pr)NH(i- | R |
| | | (morpholin- | | | pr) | |
| | | 4-yl) | | | | |
| | | eth-1-yl | | | | |
| OCH₃ | H | CH₃ | CO₂H | O | adamantan-1- | |
| | | | | | yloxy | |
| OCH₃ | H | CH₃ | CO₂H | O | —CH(i-Pr)NH₂ | R |
| OCH₃ | H | CH₃ | —CO₂Me | O | 2-(2-(2-phenyl- | S |
| | | | | | eth-1- | |
| | | | | | ylcarbonyl)-2- | |
| | | | | | aza-bicyclo | |
| | | | | | [2.2.2]- | |
| | | | | | octan-1-yl | |
| OCH₃ | H | CH₃ | CO₂H | O | t-butoxy | |
| OCH₃ | H | CH₃ | CO₂H | O | i-Propoxy | |
| OCH₃ | H | CH₃ | CO₂H | O | 1-hydroxy-1- | |
| | | | | | methyl-eth-1-yl | |
| OCH₃ | H | CH₃ | CO₂H | O | 4-(t-butoxycarbonyl)- | |
| | | | | | tetrahydropyran- | |
| | | | | | 4-yl | |
| OCH₃ | H | CH₃ | CO₂H | O | indazol-3-yl | |
| OCH₃ | H | CH₃ | CO₂H | O | (2-OMe, 4- | |
| | | | | | NH₂, | |
| | | | | | 5-Cl)phenyl | |
| OCH₃ | H | CH₃ | CO₂H | O | —CH(i-Pr) | S |
| | | | | | NHcyclohexyl | |
| NH₂ | H | CH₃ | CO₂H | O | t-butoxy | |
| OCH₃ | H | CH₃ | CO₂H | O | (2-OH) | |
| | | | | | pyridin-3-yl | |
| OCH₃ | H | CH₃ | —C(═O) | O | (2,6-Cl₂)phenyl | |
| | | | NHSO₂ | | | |
| | | | Me | | | |
| OCH₃ | H | CH₃ | CO₂H | O | (1-Me)-1H- | |
| | | | | | pyridin-2-one- | |
| | | | | | 3-yl | |
| OCH₃ | H | (2-OH) | —CH₂OH | O | (2,6-Cl₂)phenyl | |
| | | eth-1-yl | | | | |
| OCH₃ | H | CH₃ | CO₂H | O | (1- | d |
| | | | | | Boc)pyrrolidin- | |
| | | | | | 2-yl | |
| (4-Me) | H | t-Bu | CO₂H | O | (2,6-Cl₂)phenyl | |
| phenoxy | | | | | | |
| NH₂ | H | Ph | CO₂H | O | (2,6-Cl₂)phenyl | |
| OCH₃ | H | CH₃ | CO₂H | O | piperidin-4-yl | |
| OCH₃ | H | (2-OH) | —C(═O) | O | (2,6-Cl₂)phenyl | |
| | | eth-1-yl | O(CH₂)₂ | | | |
| | | | OH | | | |
| OCH₃ | H | CH₃ | CO₂H | O | (3-Cl) | |
| | | | | | thiophen-2-yl | |
| OCH₃ | H | CH₃ | CO₂H | O | thiophen-2- | |
| | | | | | ylmethoxy | |
| OCH₃ | H | CH₃ | CO₂H | O | (N-t-butoxy | |
| | | | | | carbonyl) | |
| | | | | | piperidin-4-yl | |
| OCH₃ | H | CH₃ | CO₂H | O | (2,4,6- | |
| | | | | | Me₃)benzyloxy | |
| OCH₃ | H | CH₃ | CO₂H | O | (2,6- | |
| | | | | | Cl₂)benzyloxy | |
| OCH₃ | H | CH₃ | CO₂H | O | 3H- | |
| | | | | | imidazol-4-yl | |
| OCH₃ | H | CH₃ | CO₂H | O | 3,3-Me₂-but-1- | |
| | | | | | yl | |
| OCH₃ | H | CH₃ | —CO₂CH₂ | O | (2,6-Cl₂)phenyl | |
| | | | tBu | | | |
| OCH₃ | H | CH₃ | CO₂H | O | cyclohexyloxy | |
| OCH₃ | H | CH₃ | CO₂H | O | (1-Ph)eth-1- | R |
| | | | | | oxy | |
| OCH₃ | H | 2- | CO₂H | O | —CH(i-Pr)NH₂ | R |
| | | (morpholin- | | | | |
| | | 4-yl)eth- | | | | |
| | | 1-yl | | | | |
| OCH₃ | H | CH₃ | CO₂H | O | —CH(Me)(2,5- | d |
| | | | | | Me₂, 4- | |
| | | | | | phenylcarbonyl)- | |
| | | | | | pyrrol-1-yl | |
| OCH₃ | H | (2-OH) | —C(═O) | O | O(t-Bu) | |
| | | eth-1-yl | O(CH₂)₂ | | | |
| | | | OH | | | |
| OCH₃ | H | CH₃ | CO₂H | O | —CH(i-Pr)-2,5- | R |
| | | | | | dimethyl | |
| | | | | | pyrrol-1-yl | |
| OCH₃ | H | CH₃ | CO₂H | O | —CH(i-Pr)-2,5- | S |
| | | | | | dimethyl | |
| | | | | | pyrrol-1-yl | |
| OCH₃ | H | CH₃ | CO₂H | O | —C(Me₂)(t- | |
| | | | | | butoxycarbonylamino | |
| OCH₃ | H | CH₃ | CO₂H | O | 1-hydroxy- | |
| | | | | | cycloprop-1-yl | |
| OCH₃ | H | CH₃ | CO₂H | O | —C(Me₂)(i- | |
| | | | | | propylamino) | |
| OCH₃ | H | CH₃ | CO₂H | O | cyclohexylamino | |
| OCH₃ | H | CH₃ | CO₂H | O | —C(Me₂)(1,4- | |
| | | | | | dioxa- | |
| | | | | | spiro[4.5]dec- | |
| | | | | | 8-ylamino) | |
| OCH₃ | H | CH₃ | CO₂H | O | —C(Me₂) | |
| | | | | | (methylamino) | |
| OCH₃ | H | CH₃ | CO₂H | O | 1-(t- | |
| | | | | | butoxycarbonylamino)- | |
| | | | | | cyclohex-1-yl | |
| OCH₃ | H | CH₃ | CO₂H | O | 1-(t- | |
| | | | | | butoxycarbonylamino)- | |
| | | | | | cyclopent-1-yl | |
| OCH₃ | H | CH₃ | CO₂H | O | 1-(1,4-dioxa- | |
| | | | | | spiro[4.5]dec- | |
| | | | | | 8-ylamino)- | |
| | | | | | cycloprop-1-yl | |
| OCH₃ | H | CH₃ | CO₂H | O | 1- | |
| | | | | | (cyclopentylamino)- | |
| | | | | | cycloprop- | |
| | | | | | 1-yl | |
| OCH₃ | H | CH₃ | CO₂H | O | 1- | |
| | | | | | (diethylamino)- | |
| | | | | | cycloprop-1-yl | |
| OCH₃ | H | CH₃ | CO₂H | O | 1-(methyl | |
| | | | | | carbonylamino)- | |
| | | | | | cycloprop-1- | |
| | | | | | yl | |
| OCH₃ | H | —CH₂ | CO₂H | O | (2,6-Cl₂)phenyl | |
| | | C(═O)Me | | | | |
| OCH₃ | H | CH₃ | CO₂H | O | —C(Me₂) | |
| | | | | | NHC(═O)NH₂ | |
| OCH₃ | H | (2-OH) | —C(═O)O | | t-butoxy | |
| | | eth-1-yl | (CH₂)₂OH | | | |
| NH₂ | H | CH₃ | —C(═O)O | | (2,6-Cl₂)phenyl | |
| | | | (CH₂)₂OH | | | |

and

| | | | | | |
|---|---|---|---|---|---|
| OCH₃ | H | CH₃ | CO₂H | O | 1-(phenyl |
| | | | | | methoxy)-cycloprop- |
| | | | | | 1-yl |

42. The compound of claim 1 wherein the compounds are selected from the group consisting of:

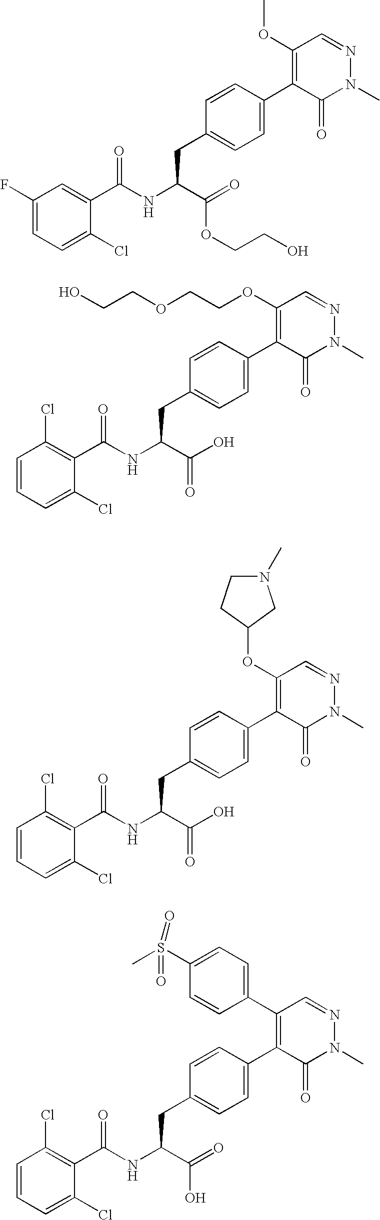

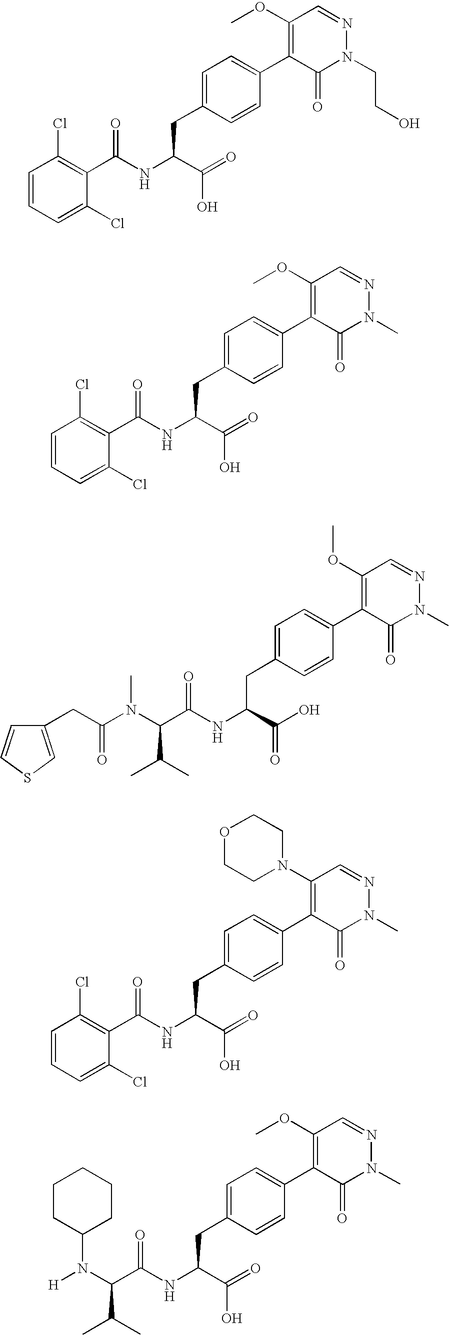

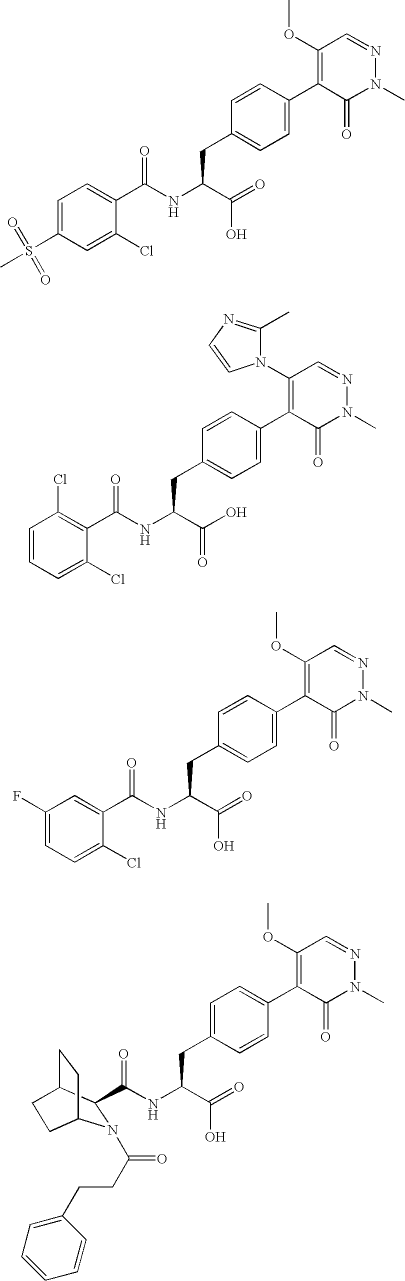

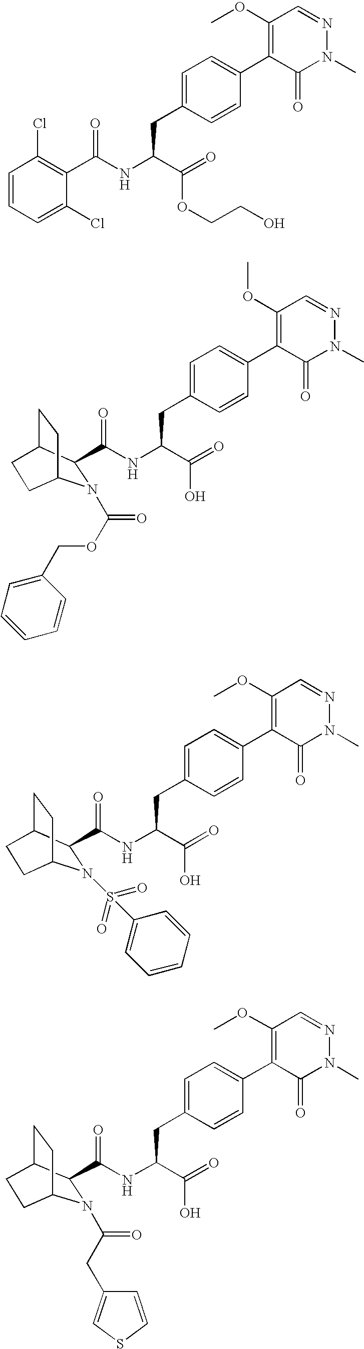

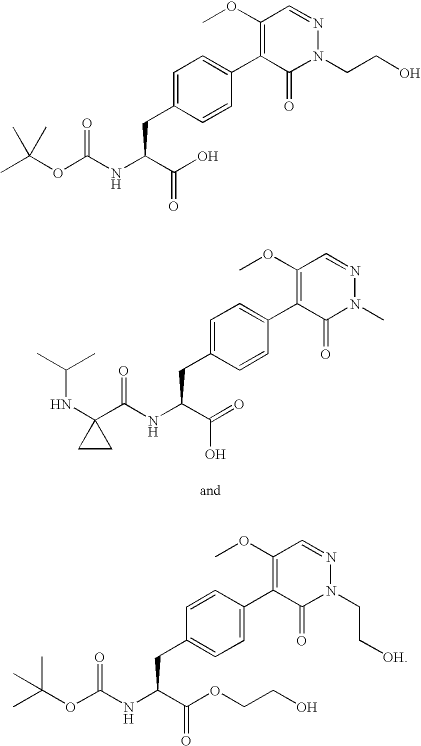

43. A composition comprising the compound of claim 1 and a pharmaceutically acceptable carrier.

44. A method of making a composition comprising admixing the compound of claim 1 and a pharmaceutically acceptable carrier.

45. A method for treating or ameliorating an α4 integrin mediated disorder in a subject in need thereof comprising administering to the subject a therapeutically effective amount of the compound of claim 1.

46. A method for treating or ameliorating an α4 integrin mediated disorder in a subject in need thereof comprising administering to the subject a therapeutically effective amount of the compound of claim 34.

47. The method of claim 45 wherein the disorder is selected from the group consisting of multiple sclerosis, asthma, allergic rhinitis, allergic conjunctivitis, inflammatory lung diseases, rheumatoid arthritis, septic arthritis, type I diabetes, organ transplantation rejection, restenosis, autologous bone marrow transplantation, inflammatory sequelae of viral infections, myocarditis, inflammatory bowel disease, toxic and immune-based nephritis, contact dermal hypersensitivity, psoriasis, tumor metastasis, atherosclerosis and hepatitis.

48. The method of claim 46 wherein the disorder is selected from the group consisting of multiple sclerosis, asthma, allergic rhinitis, allergic conjunctivitis, inflammatory lung diseases, rheumatoid arthritis, septic arthritis, type I diabetes, organ transplantation rejection, restenosis, autologous bone marrow transplantation, inflammatory sequelae of viral infections, myocarditis, inflammatory bowel disease, toxic and immune-based nephritis, contact dermal hypersensitivity, psoriasis, tumor metastasis, atherosclerosis and hepatitis.

49. The method of claim 47 herein the inflammatory bowel disease is selected from the group consisting of including ulcerative colitis and Crohn's disease.

50. The method of claim 48 herein the inflammatory bowel disease is selected from the group consisting of including ulcerative colitis and Crohn's disease.

51. The method of claim 47 wherein the therapeutically effective amount of the compound of claim 1 is from about 0.001 mg/kg/day to about 1000 mg/kg/day.
